SEQUENCE LISTING (96 sequences)

SEQ ID NO: 1 - protein, 71 aa, Cardiovirus Theiler′s-like cardiovirus
MACKHGYPLMCPLCTALDKTSDGLFTLLFDNEWYPTDLLTVDLEDEVFYPDDPHMEWTDLPLIQDIEMEPQ

SEQ ID NO: 2 - protein, 67 aa, Cardiovirus Encephalomyocarditis virus
MATTMEQETCAHSLTFEECPKCSALQYRNGFYLLKYDEEWYPEELLTDGEDDVFDPELDMEVVFELQ

SEQ ID NO: 3 - protein, 127 aa, Enterovirus Enterovirus C
NYHLATQDDLQNAVNVMWSRDLLVTESRAQGTDSIARCNCNAGVYYCESRRKYYPVSFVGPTFQYMEANNYYPARYQSHMLIGHGFASPGDCGGILRCHHGVIGIITAGGEGLVAFSDIRDLYAYEE

SEQ ID NO: 4 - protein, 219 aa, Erbovirus Equine rhinitis B virus 1
MVTMAGNMICNVFAGLATEICSPKQGPLLDNELPLPLELAEFPNKDNNCWVAALSHYYTLCDVTNHVTKVTPTTSGIRYYLTAWQSILQTDLFNGYYPAAFAVETGLCHGPFPMQQHGYVRNATSHPYNFCLCSEPVPGEDYWHAVVKVDLSRTEARVDKWLCIDDDRMYLSGPPTRVKLASSYKIPTWIESLAQFCLQLHPVQHRRTLANSLRNEQCR

SEQ ID NO: 5 - protein, 67 aa, Cardiovirus Encephalomyocarditis virus
MATTMEQEICAHSMTFEECPKCSALQYRNGFYLLKYDEEWYPEESLTDGEDDVFDPDLDMEVVFETQ

SEQ ID NO: 6 - protein, 71 aa, Cardiovirus Cardiovirus B
MACKHGYPFLCPLCTAIDTTHDGSFTLLIDNEWYPTDLLTVDLDDDVFHPDDSVMEWTDLPLIQDVVMEPQ

SEQ ID NO: 7 - protein, 149 aa, Enterovirus Enterovirus C
GFGHQNKAVYTAGYKICNYHLATQDDLQNAVNVMWSRDLLVTESRAQGTDSIARCNCNAGVYYCESRRKYYPVSFVGPTFQYMEANNYYPARYQSHMLIGHGFASPGDCGGILRCHHGVIGIITAGGEGLVAFSDIRDLYAYEEEAMEQ

SEQ ID NO: 8 - protein, 182 aa, Enterovirus Human rhinovirus 16
GPEEEFGMSIIKNNTCVVTTTNGKFTGLGIYDRILILPTHADPGSEIQVNGIHTKVLDSYDLFNKEGVKLEITVLKLDRNEKFRDIRKYIPESEDDYPECNLALVANQTEPTIIKVGDVVSYGNILLSGTQTARMLKYNYPTKSGYCGGVLYKIGQILGIHVGGNGRDGFSSMLLRSYFTEQ

SEQ ID NO: 9 - protein, 229 aa, Vesiculovirus Indiana vesiculovirus
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHTHCEGRAYLPHRMGKTPPMLNVPEHFRRPFNIGLYKGTIELTMTIYDDESLEAAPMIWDHFNSSKFSDFREKALMFGLIVEKKASGAWVLDSISHFK

SEQ ID NO: 10 - protein, 230 aa, Alphainfluenzavirus Influenza A virus
MDPNTVSSFQVDCFLWHVRKRVADQELGDAPFLDRLRRDQKSLRGRGSTLGLDIETATRAGKQIVERILKEESDEALKMTMASVPASRYLTDMTLEEMSRDWSMLIPKQKVAGPLCIRMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

SEQ ID NO: 11 - protein, 512 aa, Unknown
  Simplexvirus sp.
MATDIDMLIDLGLDLSDSDLDEDPPEPAESRRDDLESDSSGECSSSDEDMEDPHGEDGPEPILDAARPAVRPSRPEDPGVPSTQTPRPTERQGPNDPQPAPHSVWSRLGARRPSCSPEQHGGKVARLQPPPTKAQPARGGRRGRRRGRGRGGPGAADGLSDPRRRAPRTNRNPGGPRPGAGWTDGPGAPHGEAWRGSEQPDPPGGQRTRGVRQAPPPLMTLAIAPPPADPRAPAPERKAPAADTIDATTRLVLRSISERAAVDRISESFGRSAQVMHDPFGGQPFPAANSPWAPVLAGQGGPFDAETRRVSWETLVAHGPSLYRTFAGNPRAASTAKAMRDCVLRQENFIEALASADETLAWCKMCIHHNLPLRPQDPIIGTTAAVLDNLATRLRPFLQCYLKARGLCGLDELCSRRRLADIKDIASFVFVILARLANRVERGVAEIDYATLGVGVGEKMHFYLPGACMAGLIEILDTHRQECSSRVCELTASHIVAPPYVHGKYFYCNSLF

SEQ ID NO: 12 - protein, 700 aa, Artificial Sequence
  target protein sequence
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPTGGGGSGGGGSGGGGSMVSKGEELFTGVVPILVELDGDVNGHKFSVSGEGEGDATYGKLTLKFICTTGKLPVPWPTLVTTLTYGVQCFARYPDHMKQHDFFKSAMPEGYVQERTIFFKDDGNYKTRAEVKFEGDTLVNRIELKGIDFKEDGNILGHKLEYNYNSHKVYITADKQKNGIKVNFKTRHNIEDGSVQLADHYQQNTPIGDGPVLLPDNHYLSTQSKLSKDPNEKRDHMVLLEFVTAAGITLGMDELYK

SEQ ID NO: 13 - protein, 239 aa, Artificial Sequence
  target protein sequence
MVSKGEELFTGVVPILVELDGDVNGHKFSVSGEGEGDATYGKLTLKFICTTGKLPVPWPTLVTTLTYGVQCFARYPDHMKQHDFFKSAMPEGYVQERTIFFKDDGNYKTRAEVKFEGDTLVNRIELKGIDFKEDGNILGHKLEYNYNSHKVYITADKQKNGIKVNFKTRHNIEDGSVQLADHYQQNTPIGDGPVLLPDNHYLSTQSKLSKDPNEKRDHMVLLEFVTAAGITLGMDELYK

SEQ ID NO: 14 - protein, 460 aa, Artificial Sequence
  target protein sequence
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPTTGTRPLWSHPQFEK

SEQ ID NO: 15 - protein, 635 aa, Artificial Sequence
  target protein sequence
MNNFILLEEQLIKKSQQKRRTSPSNFKVRFFVLTKASLAYFEDRHGKKRTLKGSIELSRIKCVEIVKSDISIPCHYKYPFQVVHDNYLLYVFAPDRESRQRWVLALKEETRNNNSLVPKYHPNFWMDGKWRCCSQLEKLATGCAQYDPTKNASKKPLPPTPEDNRRPLWEPEETVVIALYDYQTNDPQELALRRNEEYCLLDSSEIHWWRVQDRNGHEGYVPSSYLVEKSPNNLETYEWYNKSISRDKAEKLLLDTGKEGAFMVRDSRTAGTYTVSVFTKAVVSENNPCIKHYHIKETNDNPKRYYVAEKYVFDSIPLLINYHQHNGGGLVTRLRYPVCFGRQKAPVTAGLRYGKWVIDPSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGLGGGGGGGGHHHHHHV

SEQ ID NO: 16 - protein, 500 aa, Artificial Sequence
  target protein sequence
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 17 - protein, 883 aa, Artificial Sequence
  target protein sequence
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 18 - protein, 1480 aa, Artificial Sequence
  target protein sequence
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECLFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 19 - protein, 446 aa, Artificial Sequence
  target protein sequence
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 20 - protein, 702 aa, Artificial Sequence
  target protein sequence
MTRPLLAVPGPDGGGGTGPWWAAGGRGPREVSPGAGTEVQDALERALPELQQALSALKQAGGARAVGAGLAEVFQLVEEAWLLPAVGREVAQGLCDAIRLDGGLDLLLRLLQAPELETRVQAARLLEQILVAENRDRVARIGLGVILNLAKEREPVELARSVAGILEHMFKHSEETCQRLVAAGGLDAVLYWCRRTDPALLRHCALALGNCALHGGQAVQRRMVEKRAAEWLFPLAFSKEDELLRLHACLAVAVLATNKEVEREVERSGTLALVEPLVASLDPGRFARCLVDASDTSQGRGPDDLQRLVPLLDSNRLEAQCIGAFYLCAEAAIKSLQGKTKVFSDIGAIQSLKRLVSYSTNGTKSALAKRALRLLGEEVPRPILPSVPSWKEAEVQTWLQQIGFSKYCESFREQQVDGDLLLRLTEEELQTDLGMKSGITRKRFFRELTELKTFANYSTCDRSNLADWLGSLDPRFRQYTYGLVSCGLDRSLLHRVSEQQLLEDCGIHLGVHRARILTAAREMLHSPLPCTGGKPSGDTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPT

SEQ ID NO: 21 - protein, 8 aa, Artificial Sequence
  FLAG tag
DYKDDDDK

SEQ ID NO: 22 - DNA, 46 bp, Artificial Sequence
  oligonucleotide primer
gcccgggatccaccggtcgccaccatggtgagcaagggcgaggagc

SEQ ID NO: 23 - DNA, 53 bp, Artificial Sequence
  oligonucleotide primer
agatggctggcaactagaaggcacagttacttgtacagctcgtccatgccgag

SEQ ID NO: 24 - DNA, 59 bp, Artificial Sequence
  oligonucleotide primer
cactctcggcatggacgagctgtacaagtaactgtgccttctagttgccagccatctgt

SEQ ID NO: 25 - DNA, 44 bp, Artificial Sequence
  oligonucleotide primer
cagctcctcgcccttgctcaccatggtggcgaccggtggatccc

SEQ ID NO: 26 - DNA, 53 bp, Artificial Sequence
  oligonucleotide primer
cggccagtaacgttaggggggggggattacttgtacagctcgtccatgccgag

SEQ ID NO: 27 - DNA, 57 bp, Artificial Sequence
  oligonucleotide primer
cggtaccgcgggcccgggatccaccggtcgccaccatggtgagcaagggcgaggagc

SEQ ID NO: 28 - DNA, 50 bp, Artificial Sequence
  oligonucleotide primer
ctcggcatggacgagctgtacaagtaatcccccccccctaacgttactgg

SEQ ID NO: 29 - DNA, 81 bp, Artificial Sequence
  oligonucleotide primer
acgggggaggggcaaacaacagatggctggcaactagaaggcacagctgtaactcgaaaacgacttccatgtctaattcgg

SEQ ID NO: 30 - DNA, 72 bp, Artificial Sequence
  oligonucleotide primer
cgcgggcccgggatccaccggtcgccaccatgaacaccatcaatattgccaagaacgacttttctgacatcg

SEQ ID NO: 31 - DNA, 59 bp, Artificial Sequence
  oligonucleotide primer
agatggctggcaactagaaggcacagttagggtcaggcaaatgcgaaatcggactccag

SEQ ID NO: 32 - DNA, 62 bp, Artificial Sequence
  oligonucleotide primer
cctggagtccgatttcgcatttgcctgaccctaactgtgccttctagttgccagccatctgt

SEQ ID NO: 33 - DNA, 53 bp, Artificial Sequence
  oligonucleotide primer
cgttcttggcaatattgatggtgttcatggtggcgaccggtggatcccgggcc

SEQ ID NO: 34 - DNA, 47 bp, Artificial Sequence
  oligonucleotide primer
accttggccgactctggtaatggtaatacgactcactataggaaaaa

SEQ ID NO: 35 - DNA, 45 bp, Artificial Sequence
  oligonucleotide primer
agtcagtgagcgaggaagcccaaaaaacccctcaagacccgttta

SEQ ID NO: 36 - DNA, 43 bp, Artificial Sequence
  oligonucleotide primer
aaacgggtcttgaggggttttttgggcttcctcgctcactgac

SEQ ID NO: 37 - DNA, 38 bp, Artificial Sequence
  oligonucleotide primer
tagtgagtcgtattaccattaccagagtcggccaaggt

SEQ ID NO: 38 - DNA, 60 bp, Artificial Sequence
  oligonucleotide primer
gcccgggatccaccggtcgccacctcgccaccatgaggactctgaacacctctgccatgg

SEQ ID NO: 39 - DNA, 67 bp, Artificial Sequence
  oligonucleotide primer
cttttcgaactgcgggtggctccagagcggccgcgttcccgtggttgggtgctgaccgttttgtgtg

SEQ ID NO: 40 - DNA, 60 bp, Artificial Sequence
  oligonucleotide primer
acgcggccgctctggagccacccgcagttcgaaaagtaaagcggccgcgactctagatca

SEQ ID NO: 41 - DNA, 35 bp, Artificial Sequence
  oligonucleotide primer
gtgttcagagtcctcatggtggcgaggtggcgacc

SEQ ID NO: 42 - DNA, 45 bp, Artificial Sequence
  oligonucleotide primer
atccaccggtcgccaccatgaggactctgaacacctctgccatgg

SEQ ID NO: 43 - DNA, 60 bp, Artificial Sequence
  oligonucleotide primer
tgtggtatggctgattatgatttactgtaactcgaaaacgacttccatgtctaattcggg

SEQ ID NO: 44 - DNA, 60 bp, Artificial Sequence
  oligonucleotide primer
gttttcgagttacagtaaatcataatcagccataccacatttgtagaggttttacttgct

SEQ ID NO: 45 - DNA, 40 bp, Artificial Sequence
  oligonucleotide primer
tggcagaggtgttcagagtcctcatggtggcgaccggtgg

SEQ ID NO: 46 - DNA, 49 bp, Artificial Sequence
  oligonucleotide primer
caccatcaccatcaccatgttatggccacaaccatggaacaagagactt

SEQ ID NO: 47 - DNA, 60 bp, Artificial Sequence
  oligonucleotide primer
tcttgatgagctgttcttccaggaggataaagttgttcatggtggcgaccggtggatccc

SEQ ID NO: 48 - DNA, 60 bp, Artificial Sequence
  oligonucleotide primer
cgggcccgggatccaccggtcgccaccatgaacaactttatcctcctggaagaacagctc

SEQ ID NO: 49 - DNA, 49 bp, Artificial Sequence
  oligonucleotide primer
aagtctcttgttccatggttgtggccataacatggtgatggtgatggtg

SEQ ID NO: 50 - DNA, 26 bp, Artificial Sequence
  oligonucleotide primer
ctctcggcatggacgagctgtacaag

SEQ ID NO: 51 - DNA, 29 bp, Artificial Sequence
  oligonucleotide primer
ttacttgtacagctcgtccatgccgagag

SEQ ID NO: 52 - DNA, 55 bp, Artificial Sequence
  oligonucleotide primer
tgcgcgcaagtctcttgttccatggttgtggccatggtggcgaccggtggatccc

SEQ ID NO: 53 - DNA, 36 bp, Artificial Sequence
  oligonucleotide primer
cccgaattagacatggaagtcgttttcgagttacag

SEQ ID NO: 54 - DNA, 49 bp, Artificial Sequence
  oligonucleotide primer
gggatccaccggtcgccaccatggccacaaccatggaacaagagacttg

SEQ ID NO: 55 - DNA, 36 bp, Artificial Sequence
  oligonucleotide primer
ctgtaactcgaaaacgacttccatgtctaattcggg

SEQ ID NO: 56 - DNA, 40 bp, Artificial Sequence
  oligonucleotide primer
tctcttgttccatggttgtggccatggtggcgaccggtgg

SEQ ID NO: 57 - DNA, 60 bp, Artificial Sequence
  oligonucleotide primer
acgtggttttcctttgaaaaacacgatgataaatgaggactctgaacacctctgccatgg

SEQ ID NO: 58 - DNA, 54 bp, Artificial Sequence
  oligonucleotide primer
gcagaggtgttcagagtcctcatttatcatcgtgtttttcaaaggaaaaccacg

SEQ ID NO: 59 - DNA, 45 bp, Artificial Sequence
  oligonucleotide primer
agtcgttttcgagttacagtaatcccccccccctaacgttactgg

SEQ ID NO: 60 - DNA, 52 bp, Artificial Sequence
  oligonucleotide primer
ccagtaacgttaggggggggggattactgtaactcgaaaacgacttccatgt

SEQ ID NO: 61 - DNA, 39 bp, Artificial Sequence
  oligonucleotide primer
ccaccggtcgccaccatggccacaaccatggaacaagag

SEQ ID NO: 62 - DNA, 60 bp, Artificial Sequence
  oligonucleotide primer
gatggtgtcccccgccacctccgccacctccaagtcctgattctgcaatttcagccagtt

SEQ ID NO: 63 - DNA, 90 bp, Artificial Sequence
  oligonucleotide primer
aattgcagaatcaggacttggaggtggcggaggtggcgggggacaccatcaccatcaccatgtttaatcccccccccctaacgttactgg

SEQ ID NO: 64 - DNA, 50 bp, Artificial Sequence
  oligonucleotide primer
ttataggcggacagcagcagggtcagcaccatggtggcgaggtggcgacc

SEQ ID NO: 65 - DNA, 60 bp, Artificial Sequence
  oligonucleotide primer
cggccgctcgattacaaggatgacgacgataaggtttaaagcggccgcgactctagatca

SEQ ID NO: 66 - DNA, 60 bp, Artificial Sequence
  oligonucleotide primer
taaaccttatcgtcgtcatccttgtaatcgagcggccgcgttgtagggcccatgggggcg

SEQ ID NO: 67 - DNA, 60 bp, Artificial Sequence
  oligonucleotide primer
gcgggcccgggatccaccggtcgccacctcgccaccatggtgctgaccctgctgctgtcc

SEQ ID NO: 68 - DNA, 55 bp, Artificial Sequence
  oligonucleotide primer
ccctgtcttcatggggcgagtatatgaccccagggccggaggtggcggaggtggc

SEQ ID NO: 69 - DNA, 50 bp, Artificial Sequence
  oligonucleotide primer
ggagggtcagcagggtcagcctggaggccatggtggcgaccggtggatcc

SEQ ID NO: 70 - DNA, 56 bp, Artificial Sequence
  oligonucleotide primer
cggtaccgcgggcccgggatccaccggtcgccaccatggcctccaggctgaccctg

SEQ ID NO: 71 - DNA, 52 bp, Artificial Sequence
  oligonucleotide primer
tggtgtcccccgccacctccgccacctccggccctggggtcatatactcgcc

SEQ ID NO: 72 - DNA, 47 bp, Artificial Sequence
  oligonucleotide primer
aattgcagaatcaggacttggaggtggcggaggtggcgggggacacc

SEQ ID NO: 73 - DNA, 39 bp, Artificial Sequence
  oligonucleotide primer
cataatcagccataccacatttgtagaggttttacttgc

SEQ ID NO: 74 - DNA, 28 bp, Artificial Sequence
  oligonucleotide primer
tacttgtacagctcgtccatgccgagag

SEQ ID NO: 75 - DNA, 28 bp, Artificial Sequence
  oligonucleotide primer
ctctcggcatggacgagctgtacaagta

SEQ ID NO: 76 - DNA, 39 bp, Artificial Sequence
  oligonucleotide primer
gcaagtaaaacctctacaaatgtggtatggctgattatg

SEQ ID NO: 77 - DNA, 63 bp, Artificial Sequence
  oligonucleotide primer
tcctctctgcttctagaataaatcataatcagccataccacatttgtagaggttttacttgct

SEQ ID NO: 78 - DNA, 66 bp, Artificial Sequence
  oligonucleotide primer
tgtcatgaatcagtaggtccgcaaagtaaccagcgtagtgcttgtacagctcgtccatgccgagag

SEQ ID NO: 79 - DNA, 75 bp, Artificial Sequence
  oligonucleotide primer
actttgcggacctactgattcatgacattgagacaaatccagggatgaactttctacgtaagatagtgaaaaatt

SEQ ID NO: 80 - DNA, 60 bp, Artificial Sequence
  oligonucleotide primer
acctctacaaatgtggtatggctgattatgatttattctagaagcagagaggaatctttg

SEQ ID NO: 81 - DNA, 85 bp, Artificial Sequence
  oligonucleotide primer
gctggttactttgcggacctactgattcatgacattgagacaaatccagggggattcggacaccaaaacaaagcggtgtacactg

SEQ ID NO: 82 - DNA, 70 bp, Artificial Sequence
  oligonucleotide primer
aaacctctacaaatgtggtatggctgattatgatttgttccatggcttcttcttcgtaggcatacaagtc

SEQ ID NO: 83 - DNA, 81 bp, Artificial Sequence
  oligonucleotide primer
tgtctcaatgtcatgaatcagtaggtccgcaaagtaaccagcgtagtgcttgtacagctcgtccatgccgagagtgatccc

SEQ ID NO: 84 - DNA, 80 bp, Artificial Sequence
  oligonucleotide primer
gagacttgtatgcctacgaagaagaagccatggaacaaatcataatcagccataccacatttgtagaggttttacttgct

SEQ ID NO: 85 - DNA, 73 bp, Artificial Sequence
  oligonucleotide primer
catggcagaggtgttcagagtcctcatggtggcgaccggtggattcacgacacctgaaatggaagaaaaaaac

SEQ ID NO: 86 - DNA, 53 bp, Artificial Sequence
  oligonucleotide primer
attaccgccatgcattagttattaggctccggtgcccgtcagtgggcagagcg

SEQ ID NO: 87 - DNA, 67 bp, Artificial Sequence
  oligonucleotide primer
agtttttttcttccatttcaggtgtcgtgaatccaccggtcgccaccatgaggactctgaacacctc

SEQ ID NO: 88 - DNA, 56 bp, Artificial Sequence
  oligonucleotide primer
gtgcgctctgcccactgacgggcaccggagcctaataactaatgcatggcggtaat

SEQ ID NO: 89 - DNA, 67 bp, Artificial Sequence
  oligonucleotide primer
gaggccgaggccgcctcggcctctgagctaatccaccggtcgccaccatgaggactctgaacacctc

SEQ ID NO: 90 - DNA, 67 bp, Artificial Sequence
  oligonucleotide primer
ataaccgtattaccgccatgcattagttattaggtgtggaaagtccccaggctccccagcaggcaga

SEQ ID NO: 91 - DNA, 62 bp, Artificial Sequence
  oligonucleotide primer
ttcagagtcctcatggtggcgaccggtggattagctcagaggccgaggcggcctcggcctct

SEQ ID NO: 92 - DNA, 66 bp, Artificial Sequence
  oligonucleotide primer
tctgcctgctggggagcctggggactttccacacctaataactaatgcatggcggtaatacggtta

SEQ ID NO: 93 - DNA, 38 bp, Artificial Sequence
  oligonucleotide primer
ggaggtggcggaggtggcgggggacaccatcaccatca

SEQ ID NO: 94 - DNA, 64 bp, Artificial Sequence
  oligonucleotide primer
agacaacgctggccttttccagaggcgacctctgcatggtggcgaccggtggatcccgggcccg

SEQ ID NO: 95 - DNA, 65 bp, Artificial Sequence
  oligonucleotide primer
cgggcccgggatccaccggtcgccaccatgcagaggtcgcctctggaaaaggccagcgttgtctc

SEQ ID NO: 96 - DNA, 56 bp, Artificial Sequence
  oligonucleotide primer
ccccgccacctccgccacctccaagccttgtatcttgcacctcttcttctgtctcc

(19) United States
(12) Patent Application Publication
(10) Pub. No.: US 2022/0307037 A1
(43) Pub. Date: Sep. 29, 2022

(54) SYSTEMS AND METHODS FOR PROTEIN EXPRESSION

(71) Applicant: Excepgen Inc., San Francisco, CA (US)

(72) Inventors: Barbara MERTINS, San Francisco, CA (US); Thomas FOLLIARD, San Francisco, CA (US)

(21) Appl. No.: 17/761,929

(22) PCT Filed: Sep. 15, 2020
(86) PCT No.: PCT/US2020/050910

§ 371 Date: Mar. 18, 2022

Related U.S. Application Data
(60) Provisional application No. 62/970,628, filed on Feb. 5, 2020.
(60) Provisional application No. 62/901,043, filed on Sep. 16, 2019.

(51) Int. Cl. C12N 15/63; C07K 14/005

(52) U.S. Cl. CPC C12N 15/635; C07K 14/005; C12N 2710/16622; C12N 2760/14122; C12N 2760/20022; C12N 2760/20222; C12N 2770/32022; C12N 2770/32622

(57) ABSTRACT

The present disclosure provides a system for the expression of target protein in conjunction with enhancer protein. The enhancer protein may be a viral protein that blocks nucleocytoplasmic transport. Also provided are polynucleotides, vectors, and cells comprising target protein and enhancer protein nucleic acid sequences.

### CROSS REFERENCE TO RELATED APPLICATIONS

This application claims the benefit of the U.S. Provisional Patent Application Ser. No. 62/901,043 filed Sep. 16, 2019, and the U.S. Provisional Patent Application Ser. No. 62/970,628, filed Feb. 5, 2020, the contents of each of which is herein incorporated by reference in its entirety for all purposes.

### INCORPORATION OF THE SEQUENCE LISTING

The contents of the text file submitted electronically herewith are incorporated herein by reference in their entirety: A computer readable format copy of the Sequence Listing (filename: EXCI_001_02WO_SeqList_ST25.txt, date recorded Sep. 15, 2020, file size 84.8 kb).

### BACKGROUND

Recombinant expression of proteins in eukaryotic cells grown in culture has applications in scientific research and medicine. Recombinantly produced proteins (such as antibodies, enzymes, G-protein coupled receptors (GPCRs), secreted proteins, ion channels, viral proteins, and growth factors) are used within the pharmaceutical industry to develop new drugs (e.g., small molecule discovery), as therapeutics (e.g., antibodies and other biologic drugs), and as critical assets for analytical methods. In addition to their uses within the pharmaceutical industry, recombinantly produced mammalian proteins are increasingly used in the food industry (e.g., for so-called clean meat production). For many recombinant proteins, achieving expression of recombinant protein in a functional form remains challenging.

There remains an unmet need for compositions and methods useful in the production of recombinant proteins.

### SUMMARY

The present inventors have recognized that co-expression of certain enhancer proteins with a target protein improves recombinantly produced proteins. In various embodiments, the disclosed compositions and methods exhibit one or more of the following advantages over the prior art: (1) they increase protein expression (yield) of a target protein within a cell line (e.g., a eukaryotic cell line); (2) they control the regulation of the expression of a target protein; (3) they express target protein that exhibits improved properties (e.g., decreased misfolding, altered activity, incorrect posttranslational modifications, and/or toxicity); (4) they increase correct folding and/or high yield of recombinant proteins; (5) they improve performance of the downstream activation pathways (e.g. GPCR signaling); and/or (6) co-expression of the enhancer protein does not impact functionality of the target protein and/or downstream metabolism of the cell. The invention is not limited by these enumerated advantages, as some embodiments exhibit none, some, or all of these advantages.

In one aspect, the disclosure provides a system for recombinant expression of a target protein in eukaryotic cells that includes one or more vectors. The vectors (or a vector) have a first polynucleotide encoding the target protein and a second polynucleotide encoding an enhancer protein. The enhancer protein is an inhibitor of nucleocytoplasmic transport (NCT) and/or the enhancer protein is selected from the group consisting of a picornavirus leader (L) protein, a picornavirus 2A protease, a rhinovirus 3C protease, a herpes simplex virus (HSV) ICP27 protein, and a rhabdovirus matrix (M) protein. The first polynucleotide and the second polynucleotide are operatively linked to one or more promoters.

In another aspect, the disclosure provides a eukaryotic cell for expression of a target protein, where the cell includes an exogenous polynucleotide encoding an enhancer protein. The enhancer protein is an inhibitor of nucleocytoplasmic transport (NCT) and/or the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein. The exogenous polynucleotide is operatively linked to a promoter (optionally a native promoter or an exogenous promoter). In yet another aspect, the disclosure provides a method for recombinant expression of a target protein that includes introducing a polynucleotide encoding the target protein, operatively linked to a promoter, into this eukaryotic cell. In yet another aspect, the disclosure provides a method for recombinant expression of a target protein that includes introducing a vector system of the disclosure into a eukaryotic cell. In yet another aspect, the disclosure provides a cell produced by introducing of a vector system (or vector) of the disclosure into a eukaryotic cell. In yet another aspect, the disclosure provides a protein expressed by introduction of a vector system (or vector) of the disclosure into a eukaryotic cell. In yet another aspect, the disclosure provides a method for expressing a target protein in eukaryotic cells that includes introducing a polynucleotide encoding the target protein (the polynucleotide operatively linked to a promoter) into the eukaryotic cells. This method utilizes co-expression of an enhancer protein to enhance the expression level, solubility and/or activity of the target protein. The enhancer protein is an inhibitor of nucleocytoplasmic transport (NCT) and/or the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

In another aspect, the disclosure provides a method for generating an antibody against a target protein, comprising immunizing a subject with a cell or target protein produced using the systems or methods of the disclosure. In yet another aspect, the disclosure provides a method for antibody discovery by cell sorting, comprising providing a solution comprising a labeled cell or labeled target protein produced using the systems or methods of the disclosure, and a population of recombinant cells, wherein the recombinant cells express a library of polypeptides each comprising an antibody or antigen-binding fragment thereof; and sorting one or more recombinant cells from the solution by detecting recombinant cells bound to the labeled cell or the labeled target protein. In a further aspect, the disclosure provides, a method for panning a phage-display library, comprising mixing a phage-display library with a cell or target protein produced using the systems or methods of the disclosure; and purifying and/or enriching the members of the phage-display library that bind the cell or target protein.

Further aspects and embodiments are provided by the detailed disclosure that follows. The invention is not limited by this summary.

### BRIEF DESCRIPTION OF FIGURES

FIG. 1 depicts six illustrative ways of regulating gene expression in eukaryotic cells.

FIGS. 2A-2Y are schematic drawings of non-limiting, illustrative constructs: EG1, FIG. 2A; EG2, FIG. 2B; EG3 and EG4, FIG. 2C; EGS, FIG. 2D; EG6, FIG. 2E; EG7, FIG. 2F; EG8, FIG. 2G; EG9, FIG. 2H; EG10 and EG11, FIG. 2I; EG12 and EG4, FIG. 2J; EG10, FIG. 2K; EG13, FIG. 2L; EG14, FIG. 2M; EG15, FIG. 2N; EG16, FIG. 2O; EG17, FIG. 2P; EG18, FIG. 2Q; EG19, FIG. 2R; EG20, FIG. 2S; EG21, FIG. 2T; EG22, FIG. 2U; EG23, FIG. 2V; EG24, FIG. 2W; and EG25, FIG. 2X.

FIGS. 3A-3D show images from light and fluorescent microscopy of cells expressing GFP expressed using construct EG2 (CMV-GFP-IRES-L) compared to a control vector EG1. FIG. 3A: light microscopy of cells comprising EG1. FIG. 3B: fluorescence microscopy of cells comprising EG1. FIG. 3C: light microscopy of cells comprising EG2. FIG. 3D: fluorescence microscopy of cells comprising EG2. Expression of the fluorescent GFP protein from the EG2 construct demonstrates the viability of the system. Reduction of deleterious over-expression in cells comprising EG2 (FIG. 3D) compared to cells comprising EG1 (FIG. 3B) demonstrates the improved regulation of GFP expression by introduction of the L-protein. The bar in FIGS. 3A-3D represents 400 microns.

FIGS. 4A-4D show images from light and fluorescent microscopy of cells expressing GFP expressed using constructs EG3 and EG4 (T7-IRES-L-GFP and CMV-T7, respectively) compared to a control vector EG1. FIG. 4A: light microscopy of cells comprising EG1. FIG. 4B: fluorescence microscopy of cells comprising EG1. FIG. 4C: light microscopy of cells comprising EG3+EG4. FIG. 4D: fluorescence microscopy of cells comprising EG3+EG4. Expression of the fluorescent GFP protein from the EG3+EG4 constructs demonstrates the viability of the system. Reduction of expression in cells comprising EG3+EG4 (FIG. 4D) compared to cells comprising EG1 (FIG. 4B) demonstrates the improved regulation of GFP expression by introduction of the L-protein. The bar in FIGS. 4A-4D represents 400 microns.

FIGS. 5A-5D show images from fluorescent microscopy of cells expressing DRD1-GFP fusion from construct EG10 (CMV-[DRD1-GFP]) (FIG. 5A) or EG8 (CMV-[DRD1-GFP]-IRES-L) (FIG. 5C). DRD1-GFP using construct EG10 is expressed but fails to transport the receptor into the outer membrane, leading to the formation of inclusion bodies (FIG. 5B, arrow). DRD1-GFP using construct EG8 is expressed and reliably transported into the membrane resulting in a high yield of the GPCR on the outer membrane with a high quality (FIG. 5D).

FIGS. 6A-6B show images from fluorescent microscopy of cells expressing DRD1-GFP fusion protein expressed from construct EG10 (CMV-[DRD1-GFP]) (FIG. 6A) or EG12 and EG4 (T7-IRES-L-DRD1-GFP and CMV-T7, respectively) (FIG. 6B). DRD1-GFP expressed using EG10 is expressed but fails to correctly transport the receptor into the outer membrane, leading to the formation of inclusion bodies (FIG. 6A, arrow). DRD1-GFP expressed using EG12 in combination with EG4 is expressed and reliably transported into the membrane resulting in a high yield of the GPCR on the outer membrane with a high quality (FIG. 6B).

FIG. 7 shows results from an anti-CFTR Western blot. Co-expression of the L-protein and CFTR delivered as PCR product or as vector (left of a dashed line) leads to a decrease of yield but to a more homogenous sample compared to control expression of CFTR without co-expression of L-protein (right of dashed line).

FIGS. 8A-8B show results from a purification and activity test of NADase. FIG. 8A shows SDS-PAGE of NADase affinity purified using a FLAG tag. (Standard, SeeBlue2 plus; lane 2, lysate/load; lane 3, flow through; lane 4, column elution fraction 1; lane 5, column elution fraction 2; lane 6, column elution fraction 3; lane 7, column elution fraction 4; 8, resin). FIG. 8B shows a graph of NAD+ conversion activity analyzed by high-performance liquid chromatography (HPLC) using different concentrations of purified NADase.

FIG. 9A-9B show the results of His-tag purification of ITK. FIG. 9A shows SDS-PAGE of ITK affinity purified using a His tag. Lanes: lane 1, SeeBlue2 plus prestained; lane 2, 500 ng GFP; lane 3, 2 μg ITK; lane 4, 5 μg ITK; lane 5, 10 μg ITK. FIG. 9B shows Western Blot analysis after SDS-PAGE of FIG. 9A, with arrows pointing to the monomer and dimer of ITK.

FIG. 10 shows images from fluorescent microscopy of cells expressing DRD1-GFP fusion protein expressed from construct EG10 (CMV-[DRD1-GFP]) (FIG. 10A) or EG10 and EG11 (FIG. 10B). Arrow points to the inclusion bodies formed by DRD1-GFP expressed from EG10, which fails to correctly transport the receptor into the outer membrane.

FIG. 11 shows a graph showing the luminescence results from cAMP-Glo™ assay, which indicates the cAMP levels in cells expressing either E5 construct (CMV-DRD1-Strp) or E6 construct (CMV-DRD1-Strp-IRES-L) in HEK293 cells in the presence or absence of dopamine. Higher luminescence signal indicates higher functional activity of DRD1-Strep.

FIG. 12 shows images from fluorescent microscopy of cells expressing DRD1-GFP fusion protein expressed using a CMV promoter (FIG. 12A), DRD1-GFP fusion protein expressed in combination with L protein using a CMV promoter (FIG. 12B), DRD1-GFP fusion protein expressed in combination with L protein using a EF1-α promoter (FIG. 12C), and DRD1-GFP fusion protein expressed in combination with L protein using a SV40 promoter (FIG. 12D). The bottom panels show enlarged views of some cells shown in the top panels.

FIG. 13 shows images from fluorescent microscopy of HEK293 cells expressing DRD1-GFP fusion protein (FIG. 13A), DRD1-GFP fusion protein expressed in combination with L protein from EMCV (FIG. 13B), DRD1-GFP fusion protein expressed in combination with L protein from Theiler's virus (FIG. 13C), DRD1-GFP fusion protein expressed in combination with 2A protease of Polio virus (FIG. 13D) and the DRD1-GFP fusion protein expressed in combination with the M protein of vesicular stomatitis virus (FIG. 13E). The bottom panels show enlarged views of some cells shown in the top panels.

FIG. 14 shows images from fluorescent microscopy of CHO-K1 cells expressing DRD1-GFP fusion protein (FIG. 14A), and DRD1-GFP fusion protein expressed in combination with L protein from Theiler's virus (FIG. 14B). Arrow points to the inclusion bodies formed by DRD1-GFP expressed from EG10, which fails to correctly transport the receptor into the outer membrane.

FIG. 15 shows images from fluorescent microscopy of CHO-K1 cells expressing DRD1-GFP fusion protein (FIG. 15A), and DRD1-GFP fusion protein expressed in combination with L protein from EMCV (FIG. 15B). In FIG. 15A, arrow points to the inclusion bodies formed by DRD1-GFP expressed from EG10, which fails to correctly transport the receptor into the outer membrane. In FIG. 15B, arrow points to correctly localized and membrane-incorporated DRD1-GFP.

FIG. 16 shows images from SDS-PAGE analysis of ITK protein expressed in HEK293 cells purified using nickel-charged affinity resin (FIG. 16A), or size exclusion chromatography (FIG. 16B), and ITK-L fusion protein expressed in HEK293 cells purified using nickel-charged affinity resin (FIG. 16C), or size exclusion chromatography (FIG. 16D). P1 refers to the dimeric form of ITK, while P2 refers to the monomeric form of ITK.

FIG. 17A shows results from the SDS-PAGE analysis of purified ITK protein, and purified ITK protein expressed in combination with L protein in HEK293 cells. FIG. 17B shows a graph of luminescence measurement of P1 and P2 ITK purified protein samples, as indicated on SDS PAGE image.

FIG. 18 shows images from SDS-PAGE analysis of ITK protein expressed in CHO cells purified using nickel-charged affinity resin (FIG. 18A), or size exclusion chromatography (FIG. 18B), and ITK protein expressed in combination with L protein in CHO cells purified using nickel-charged affinity resin (FIG. 18C), or size exclusion chromatography (FIG. 18D). P1 refers to the dimeric form of ITK, while P2 refers to the monomeric form of ITK.

FIG. 19 shows a graph of luminescence measurement of P1 and P2 ITK protein samples expressed in combination with L protein in CHO cells, and purified using size exclusion chromatography experiment.

FIG. 20A shows the image from SDS-PAGE analysis of purified C1-Inhibitor expressed the in absence (left) or presence (right) of the enhancer L protein. FIG. 20B shows a graph depicting the concentration of functionally active C1-inhibitor present in the purified C1-inhibitor protein sample, expressed in the presence or absence the enhancer L protein, as indicated. The data were obtained using the commercial Quidel MicroVue Complement C1-Inhibitor Plus Enzyme Immunoassay, using C1-inhibitor containing healthy human plasma as a positive control (100% activity) as per manufacturer's protocol.

FIG. 21 shows the ion exchange chromatography of PSG1. Protein containing fractions (FIG. 21A, red box) were pooled and concentrated before confirming the presence of PSG1 by SDS-PAGE and Western blot (FIG. 21B, red arrow).

### DETAILED DESCRIPTION

According to the present disclosure, a vector system, vector, or eukaryotic cell is provided that is useful in co-expression of an enhancer protein with a target protein. In some embodiments, provided is a system for recombinant expression of a target protein in eukaryotic cells that includes one or more vectors. In some embodiments, the vectors (or a vector) have a first polynucleotide encoding the target protein and a second polynucleotide encoding an enhancer protein. The enhancer protein is an inhibitor of nucleocytoplasmic transport (NCT) and/or the enhancer protein is selected from the group consisting of a picornavirus leader (L) protein, a picornavirus 2A protease, a rhinovirus 3C protease, a herpes simplex virus (HSV) ICP27 protein, and a rhabdovirus matrix (M) protein. The first polynucleotide and the second polynucleotide are operatively linked to one or more promoters.

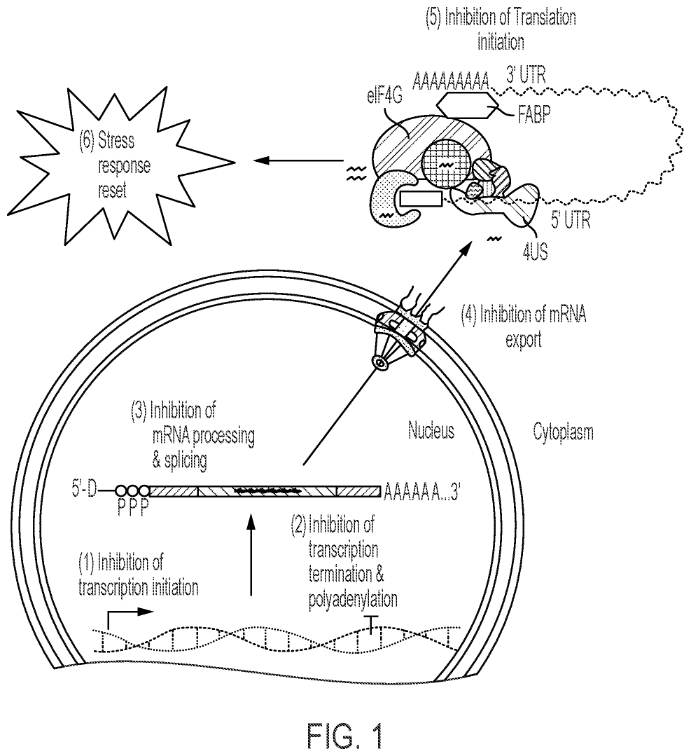

Without being bound by theory, it is believed that the compositions and methods of the disclosure prevent regulatory mechanisms of the cell from activating in response to expression of the recombinant target protein, and that this improves yields and/or functionality of the target protein. The methods and systems of the disclosure may inhibit or interfere with one or more cellular mechanisms, including but not limited to: (1) inhibition of transcription initiation, (2) inhibition of transcription termination and polyadenylation; (3) inhibition of mRNA processing and splicing, (4) inhibition of mRNA export; (5) inhibition of translation initiations; and (6) stress response (FIG. 1).

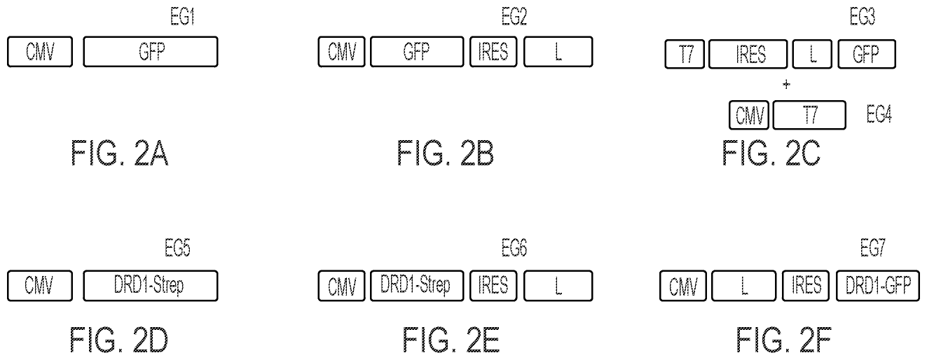

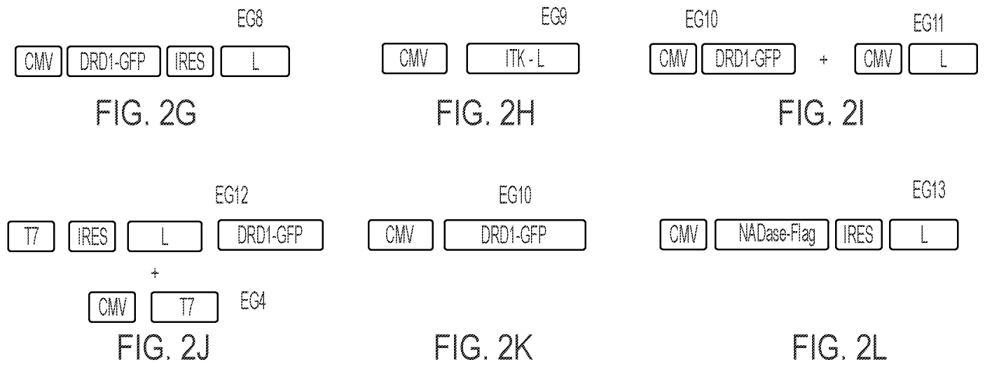

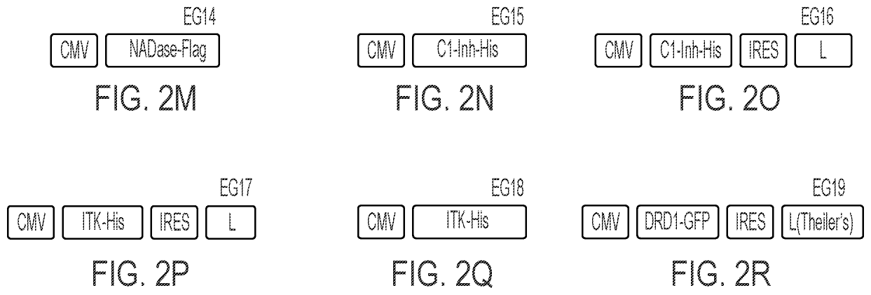

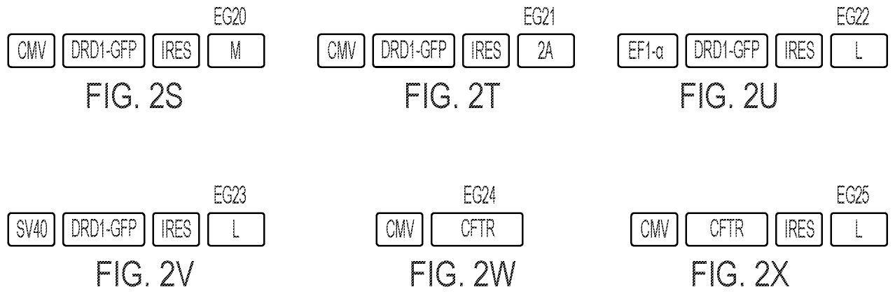

Various embodiments are depicted in FIGS. 2A-2Y and Table 1. In some embodiments, a first vector includes a polynucleotide encoding the target protein and a second vector includes a polynucleotide encoding the enhancer protein. In other embodiments, a single vector includes one or more polynucleotides encoding the target protein and the enhancer protein. The vector may comprise a single polynucleotide encoding both the target protein and the enhancer protein. In the alternative, more than one enhancer protein and/or more than one target protein are encoded by the vector or vectors.

### Polynucleotides

The present disclosure relates to recombinant polynucleotides for the expression of one or more target proteins and one or more enhancer proteins. Polynucleotides (or nucleic acids or nucleic acid molecules) may comprise one or more genes of interest and is delivered to cells (e.g., eukaryotic cells) using the compositions and methods of the present disclosure. Polynucleotides of the present disclosure may include DNA, RNA, and DNA-RNA hybrid molecules. In some embodiments, polynucleotides are isolated from a natural source; prepared in vitro, using techniques such as PCR amplification or chemical synthesis; prepared in vivo, e.g., via recombinant DNA technology; or prepared or obtained by any appropriate method. In some embodiments, polynucleotides are of any shape (linear, circular, etc.) or topology (single-stranded, double-stranded, linear, circular, supercoiled, torsional, nicked, etc.). Polynucleotides may also comprise nucleic acid derivatives such as peptide nucleic acids (PNAS) and polypeptide-nucleic acid conjugates; nucleic acids having at least one chemically modified sugar residue, backbone, internucleotide linkage, base, nucleotide, nucleoside, or nucleotide analog or derivative; as well as nucleic acids having chemically modified 5′ or 3′ ends; and nucleic acids having two or more of such modifications. Not all linkages in a polynucleotide need to be identical.

Examples of polynucleotides include without limitation oligonucleotides (including but not limited to antisense oligonucleotides, ribozymes and oligonucleotides useful in RNA interference (RNAi)), aptamers, nucleic acids, artificial chromosomes, cloning vectors and constructs, expression vectors and constructs, gene therapy vectors and constructs, rRNA, tRNA, mRNA, mtRNA, and tmRNA, and the like. In some embodiments, the polynucleotide is an in vitro transcribed (IVT) mRNA. In some embodiments, the polynucleotide is a plasmid.

A polynucleotide is said to “encode” a protein when it comprises a nucleic acid sequence that is capable of being transcribed and translated (e.g., DNA→RNA→protein) or translated (RNA→protein) in order to produce an amino acid sequence corresponding to the amino acid sequence of said protein. In vivo (e.g., within a eukaryotic cell) transcription and/or translation is performed by endogenous or exogenous enzymes. In some embodiments, transcription of the polynucleotides of the disclosure is performed by the endogenous polymerase II (polII) of the eukaryotic cell. In some embodiments, an exogenous RNA polymerase is provided on the same or a different vector. In some embodiments, the RNA polymerase is selected from a T3 RNA polymerase, a T5 RNA polymerase, a T7 RNA polymerase, and an H8 RNA polymerase.

Illustrative polynucleotides according to the present disclosure include a “first polynucleotide” encoding a target protein; a “second polynucleotide” encoding an enhancer protein; and a “coding polynucleotide” encoding one or more target proteins, one or more enhancer proteins, and/or one or more separating elements.

Target Proteins

Polynucleotides according to the present disclosure may comprise a nucleic acid sequence encoding for one or more target proteins. The nucleic acid sequence encoding the target protein is referred to as the gene of interest (“GOI”). The target protein is any protein for which expression is desired. In some embodiments, the protein is a membrane protein. In some embodiments, the expression of the protein may cause cell toxicity when expressed in a reference expression system. In some embodiments, the protein is a protein with low yield expression in traditional expression systems. In some embodiments, the expression or quality of the protein is significantly improved by expression according to the disclosed methods, e.g., in conjunction with one or more enhancer proteins. In some embodiments, the target protein is an AAV capsid protein. The AAV capsid target protein may be a native AAV capsid protein, or a mutant AAV capsid protein that comprises one or more mutations in the native AAV capsid protein sequence.

A target protein for expression through the use of the present compositions and methods may include proteins related to enzyme replacement, such as Agalsidase beta, Agalsidase alfa, Imiglucerase, Taligulcerase alfa, Velaglucerase alfa, Alglucerase, Sebelipase alpha, Laronidase, Idursulfase, Elosulfase alpha, Galsulfase, Alglucosidase alpha, Factor VIII, C3 inhibitor, Hurler and Hunter corrective factors. In some embodiments, a target protein is a biosimilar. In some embodiments, a target protein may a secreted protein, e.g., C1-Inh. In some embodiments, a target protein is an antibody. In some embodiments, the present compositions and methods are used for enzyme production. Such enzymes may be useful in the production of clinical testing kits or other diagnostic assays. In some embodiments, the present compositions and methods are used to produce therapeutic proteins. In some embodiments, the protein is a human protein and the host cell for expression is a human cell.

In some embodiments, the target protein is selected from the group consisting of Abarelix, Abatacept, Abciximab, Adalimumab, Aflibercept, Agalsidase beta, Albiglutide, Aldesleukin, Alefacept, Alemtuzumab, Alglucerase, Alglucosidase alfa, Alirocumab, Aliskiren, Alpha-1-proteinase inhibitor, Alteplase, Anakinra, Ancestim, Anistreplase, Anthrax immune globulin human, Antihemophilic Factor, Antithrombin Alfa, Antithrombin III human, Antithymocyte globulin, Anti-thymocyte Globulin (Equine), Anti-thymocyte Globulin (Rabbit), Aprotinin, Arcitumomab, Asfotase Alfa, Asparaginase, Asparaginase erwinia chrysanthemi, Atezolizumab, Autologous cultured chondrocytes, Basiliximab, Becaplermin, Belatacept, Belimumab, Beractant, Bevacizumab, Bivalirudin, Blinatumomab, Botulinum Toxin Type A, Botulinum Toxin Type B, Brentuximab vedotin, Brodalumab, Buserelin, C1 Esterase Inhibitor (Human), C1 Esterase Inhibitor , Canakinumab, Canakinumab, Capromab, Certolizumab pegol, Cetuximab, Choriogonadotropin alfa, Chorionic Gonadotropin (Human), Chorionic Gonadotropin, Coagulation factor IX, Coagulation factor VIIa, Coagulation factor X human, Coagulation Factor XIII A-Subunit, Collagenase, Conestat alfa, Corticotropin, Cosyntropin, Daclizumab, Daptomycin, Daratumumab, Darbepoetin alfa, Defibrotide, Denileukin diftitox, Denosumab, Desirudin, Dinutuximab, Dornase alfa, Drotrecogin alfa, Dulaglutide, Eculizumab, Efalizumab, Efmoroctocog alfa, Elosulfase alfa, Elotuzumab, Enfuvirtide, Epoetin alfa, Epoetin zeta, Eptifibatide, Etanercept, Evolocumab, Exenatide, Factor IX Complex (Human), Fibrinogen Concentrate (Human), Fibrinolysin aka plasmin, Filgrastim, Filgrastim-sndz, Follitropin alpha, Follitropin beta, Galsulfase, Gastric intrinsic factor, Gemtuzumab ozogamicin, Glatiramer acetate, Glucagon recombinant, Glucarpidase, Golimumab, Gramicidin D, Hepatitis A Vaccine, Hepatitis B immune globulin, Human calcitonin, Human clostridium tetani toxoid immune globulin, Human rabies virus immune globulin, Human Rho(D) immune globulin, Human Serum Albumin, Human Varicella-Zoster Immune Globulin, Hyaluronidase, Hyaluronidase, Ibritumomab, Ibritumomab tiuxetan, Idarucizumab, Idursulfase, Imiglucerase, Immune Globulin Human, Infliximab, Insulin aspart, Insulin Beef, Insulin Degludec, Insulin detemir, Insulin Glargine, Insulin glulisine, Insulin Lispro, Insulin Pork, Insulin Regular, Insulin Regular, Insulin, porcine, Insulin,isophane, Interferon Alfa-2a, Recombinant, Interferon alfa-2b, Interferon alfacon-1, Interferon alfa-n1, Interferon alfa-n9, Interferon beta-la, Interferon beta-lb, Interferon gamma-lb, Intravenous Immunoglobulin, Ipilimumab, Ixekizumab, Laronidase, Lenograstim, Lepirudin, Leuprolide, Liraglutide, Lucinactant, Lutropin alfa, Lutropin alfa, Mecasermin, Menotropins, Mepolizumab, Epoetin beta, Metreleptin, Muromonab, Natalizumab, alpha interferon, Necitumumab, Nesiritide, Nivolumab, Obiltoxaximab, Obinutuzumab, Ocriplasmin, Ofatumumab, Omalizumab, Oprelvekin, OspA lipoprotein, Oxytocin, Palifermin, Palivizumab, Pancrelipase, Panitumumab, Pembrolizumab, Pertuzumab, Poractant alfa, Pramlintide, Preotact, Protein S human, Ramucirumab, Ranibizumab, Rasburicase, Raxibacumab, Reteplase, Rilonacept, Rituximab, Romiplostim, Sacrosidase, Salmon Calcitonin, Sargramostim, Satumomab Pendetide, Sebelipase alfa, Secretin, Secukinumab, Sermorelin, Serum albumin, Serum albumin iodonated, Siltuximab, Simoctocog Alfa, Sipuleucel-T, Somatotropin Recombinant, Somatropin recombinant, Streptokinase, Sulodexide, Susoctocog alfa, Taliglucerase alfa, Teduglutide, Teicoplanin, Tenecteplase, Teriparatide, Tesamorelin, Thrombomodulin alfa, Thymalfasin, Thyroglobulin, Thyrotropin Alfa, Thyrotropin Alfa, Tocilizumab, Tositumomab, Trastuzumab, Tuberculin Purified Protein Derivative, Turoctocog alfa, Urofollitropin, Urokinase, Ustekinumab, Vasopressin, Vedolizumab, and Velaglucerase alfa.

In some embodiments, the target protein is, without limitation, a soluble protein, a secreted protein, or a membrane protein. In some embodiments, the target protein is, without limitation, Dopamine receptor 1 (DRD1), Cystic fibrosis transmembrane conductance regulator (CFTR), C1 esterase inhibitor (C1-Inh), IL2 inducible T cell kinase (ITK), or an NADase. In some embodiments, the NADase is SARM1. In some embodiments, the SARM1 is a deletion variant that represents the mature protein.

In some embodiments, a target protein is a membrane protein. Illustrative membrane proteins include ion channels, gap junctions, ionotropic receptors, transporters, integral membrane proteins such as cell surface receptors (e.g. G-protein coupled receptors (GPCRs), tyrosine kinase receptors, integrins and the like), proteins that shuttle between the membrane and cytosol in response to signaling (e.g. Ras, Rac, Raf, Gα subunits, arresting, Src and other effector proteins), and the like. In some embodiments, the membrane protein is a G protein-coupled receptor. In some embodiments, the target protein is a seven-(pass)-transmembrane domain receptor, 7TM receptor, heptahelical receptor, serpentine receptor, or G protein-linked receptor (GPLR). In some embodiments, the target protein is a Class A GPCR, Class B GPCR, Class C GPCR, Class D GPCR, Class E GPCR, or Class F GPCR. In some embodiments, the target protein is a Class 1 GPCR, Class 2 GPCR, Class 3 GPCR, Class 4 GPCR, Class 5 GPCR, or Class 6 GPCR. In some embodiments, the target protein is a Rhodopsin-like GPCR, a Secretin receptor family GPCR, a Metabotropic glutamate/pheromone GPCR, a Fungal mating pheromone receptor, a Cyclic AMP receptor, or a Frizzled/Smoothened GPCR.

In some embodiments, a target protein is a nucleosidase, an NAD+ nucleosidase, a hydrolase, a glycosylase, a glycosylase that hydrolyzes N-glycosyl compounds, an NAD+ glycohydrolase, an NADase, a DPNase, a DPN hydrolase, an NAD hydrolase, a diphosphopyridine nucleosidase, a nicotinamide adenine dinucleotide nucleosidase, an NAD glycohydrolase, an NAD nucleosidase, or a nicotinamide adenine dinucleotide glycohydrolase. In some embodiments, the target protein is an enzyme that participates in nicotinate and nicotinamide metabolism and calcium signaling pathway.

In some embodiments, the present disclosure provides a protein expressed by introduction of a vector system (or vector) of the disclosure into a eukaryotic cell. In some embodiments, the present disclosure provides a target protein produced by eukaryotic cells comprising polynucleotides of the disclosure.

Enhancer Proteins

The present disclosure relates to the co-expression of target proteins and enhancer proteins. In some embodiments, the enhancer proteins may improve one or more aspects of target protein expression, including but not limited to yield, quality, folding, posttranslational modification, activity, localization, and downstream activity, or may reduce one or more of misfolding, altered activity, incorrect posttranslational modifications, and/or toxicity.

In some embodiments, an enhancer protein is a nuclear pore blocking viral protein. In some embodiments, the enhancer protein is a native or synthetic peptide that is capable of blocking the nuclear pore, thereby inhibiting nucleocytoplasmic transport (“NCT”). In some embodiments, the enhancer protein is a viral protein. In some aspects, the viral protein is an NCT inhibitor.

In some embodiments, the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

The enhancer protein is a functional variant of any of the proteins disclosed herein. As used herein, the term “functional variant” refers to a protein that is homologous to an original protein and/or shares substantial sequence similarity to that original protein (e.g., more than 30%, 40%, 50%, 60%, 70%, 80%, 85% 90%, 95%, or 99% sequence identity) and shares one or more functional characteristics of the original protein. For example, a functional variant of an enhancer protein that is an NCT inhibitor retains the ability to inhibit NCT.

In some embodiments, the enhancer protein is a leader (L) protein from a picornavirus or a functional variant thereof. In some embodiments, the enhancer protein is a leader protein from the Cardiovirus, Hepatovirus, or Aphthovirus genera. For example, the enhancer protein may be from Bovine rhinitis A virus, Bovine rhinitis B virus, Equine rhinitis A virus, Foot-and-mouth disease virus, Hepatovirus A, Hepatovirus B, Marmota himalayana hepatovirus, Phopivirus, Cardiovirus A, Cardiovirus B, Theiler's Murine encephalomyelitis virus (TMEV), Vilyuisk human encephalomyelitis virus (VHEV), Theiler-like rat virus (TRV), or Saffold virus (SAF-V).

In some embodiments, the enhancer protein is the L protein of Theiler's virus or a functional variant thereof. In some embodiments, the L protein shares at least 90% identity to SEQ ID NO: 1. In some embodiments, the enhancer protein may comprise, consist of, or consist essentially of SEQ ID NO: 1. The enhancer protein may share at least 70%, 75%, 80%, 85%, 90%, 95%, 98%, 99% or 100% identity to SEQ ID NO: 1.

In some embodiments, the L protein is the L protein of Encephalomyocarditis virus (EMCV) or a functional variant thereof. In some embodiments, the L protein may share at least 90% identity to SEQ ID NO: 2. In some embodiments, the enhancer protein may comprise, consist of, or consist essentially of SEQ ID NO: 2. The enhancer protein may share at least 70%, 75%, 80%, 85%, 90%, 95%, 98%, 99% or 100% identity to SEQ ID NO: 2.

In some embodiments, the L protein is selected from the group consisting of the L protein of poliovirus, the L protein of HRV16, the L protein of mengo virus, and the L protein of Saffold virus 2 or a functional variant thereof.

In some embodiments, the enhancer protein is a picornavirus 2A protease or a functional variant thereof. In some embodiments, the enhancer protein is a 2A protease from Enterovirus, Rhinovirus, Aphtovirus, or Cardiovirus.

In some embodiments, the enhancer protein is a rhinovirus 3C protease or a functional variant thereof. In some embodiments, the enhancer protein is a Picornain 3C protease. In some embodiments, the enhancer protein is a 3C protease from Enterovirus, Rhinovirus, Aphtovirus, or Cardiovirus. For example, in some non-limiting embodiments, the enhancer protein is a 3C protease from Poliovirus, Coxsackievirus, Rhinovirus, Foot-and-mouth disease virus, or Hepatovirus A.

In some embodiments, the enhancer protein is a coronavirus ORF6 protein or a functional variant thereof. In some embodiments, the enhancer protein is a viral protein that disrupts nuclear import complex formation and/or disrupts STAT1 transport into the nucleus.

In some embodiments, the enhancer protein is an ebolavirus VP24 protein or a functional variant thereof. In some embodiments, the enhancer protein is an ebolavirus VP40 protein or VP35 protein. In some embodiments, the enhancer protein is a viral protein that binds to the importin protein karyopherin-α (KPNA). In some embodiments, the enhancer protein is a viral protein that inhibits the binding of STAT1 to KPNA.

In some embodiments, the enhancer protein is a Venezuelan equine encephalitis virus (VEEV) capsid protein or a functional variant thereof. In some embodiments, the enhancer protein is a viral capsid protein that interacts with the nuclear pore complex.

In some embodiments, the enhancer protein is a herpes simplex virus (HSV) ICP27 protein or a functional variant thereof. In some embodiments, the enhancer protein is an HSV ORF57 protein.

In some embodiments, the enhancer protein is a rhabdovirus matrix (M) protein or a functional variant thereof. In some embodiments, the enhancer protein is an M protein from Cytorhabdovirus, Dichorhavirus, Ephemerovirus, Lyssavirus, Novirhabdovirus, Nucleorhabdovirus, Perhabdovirus, Sigmavirus, Sprivivirus, Tibrovirus, Tupavirus, Varicosavirus, or Vesiculovirus.

In some embodiments, an enhancer protein is selected from the proteins listed in Table 1 or functional variants thereof. The polynucleotide encoding the enhancer protein may encode an amino acid sequence at least 70%, 75%, 80%, 85%, 90%, 95%, 98%, 99% or 100% identical to an amino acid sequence listed in Table 1. The amino acid sequence of the enhancer protein may be at least 70%, 75%, 80%, 85%, 90%, 95%, 98%, 99% or 100% identical to an amino acid sequence listed in Table 1. The amino acid sequence of the enhancer protein may be at least 70%, 75%, 80%, 85%, 90%, 95%, 98%, 99% or 100% identical to the amino acid sequence of SEQ ID NO: 1, 2, 3, 4, 5, 6, 7, 8, 9, 10, or 11. In some embodiments, an enhancer protein may have an amino acid sequence comprising, consisting of, or consisting essentially of one of the amino acid sequences listed in Table 1. In some embodiments, an enhancer protein may have an amino acid sequence comprising, consisting of, or consisting essentially of the amino acid sequence of SEQ ID NO: 1, 2, 3, 4, 5, 6, 7, 8, 9, 10, or 11.

TABLE 1
Illustrative enhancer proteins

| Nuclear pore | | | |
|---|---|---|---|
| blocking viral | | | |
| protein | Origin | Family | Amino acid sequence |
| Leader protein | Theiler's virus | Picornaviridae | MACKHGYPLMCPLCTALDK |
| | | | TSDGLFTLLFDNEWYPTDLLT |
| | | | VDLEDEVFYPDDPHMEWTDL |
| | | | PLIQDIEMEPQ |
| | | | (SEQ ID NO: 1) |
| Leader protein | EMCV | Picornaviridae | MATTMEQETCAHSLTFEECP |
| | | | KCSALQYRNGFYLLKYDEEW |
| | | | YPEELLTDGEDDVFDPELDM |
| | | | EVVFELQ |
| | | | (SEQ ID NO: 2) |
| Leader protein | Poliovirus | Picornaviridae | NYHLATQDDLQNAVNVMWS |
| | (Enterovirus C) | | RDLLVTESRAQGTDSIARCNC |
| | | | NAGVYYCESRRKYYPVSFVG |
| | | | PTFQYMEANNYYPARYQSH |
| | | | MLIGHGFASPGDCGGILRCHH |
| | | | GVIGIITAGGEGLVAFSDIRDL |
| | | | YAYEE |
| | | | (SEQ ID NO: 3) |
| Leader protein | Equine rhinitis | Picornaviridae | MVTMAGNMICNVFAGLATEI |
| | B virus 1 | | CSPKQGPLLDNELPLPLELAE |
| | | | FPNKDNNCWVAALSHYYTL |
| | | | CDVTNHVTKVTPTTSGIRYYL |
| | | | TAWQSILQTDLFNGYYPAAF |
| | | | AVETGLCHGPFPMQQHGYVR |
| | | | NATSHPYNFCLCSEPVPGEDY |
| | | | WHAVVKVDLSRTEARVDKW |
| | | | LCIDDDRMYLSGPPTRVKLAS |
| | | | SYKIPTWIESLAQFCLQLHPV |
| | | | QHRRTLANSLRNEQCR |
| | | | (SEQ ID NO: 4) |
| Leader protein | Mengo virus | Picornaviridae | MATTMEQEICAHSMTFEECP |
| | (Cardiovirus) | | KCSALQYRNGFYLLKYDEEW |
| | | | YPEESLTDGEDDVFDPDLDM |
| | | | EVVFETQ |
| | | | (SEQ ID NO: 5) |
| Leader protein | Saffold virus 2 | Picornaviridae | MACKHGYPFLCPLCTAIDTT |
| | (Cardiovirus) | | HDGSFTLLIDNEWYPTDLLTV |
| | | | DLDDDVFHPDDSVMEWTDL |
| | | | PLIQDVVMEPQ |
| | | | (SEQ ID NO: 6) |
| 2A protease | Poliovirus | Picornaviridae | GFGHQNKAVYTAGYKICNY |
| | (Enterovirus C) | | HLATQDDLQNAVNVMWSRD |
| | | | LLVTESRAQGTDSIARCNCNA |
| | | | GVYYCESRRKYYPVSFVGPT |
| | | | FQYMEANNYYPARYQSHMLI |
| | | | GHGFASPGDCGGILRCHHGVI |
| | | | GIITAGGEGLVAFSDIRDLYA |
| | | | YEEEAMEQ |
| | | | (SEQ ID NO: 7) |
| 3C protease | HRV16 | Picornaviridae | GPEEEFGMSIIKNNTCVVTTT |
| | | | NGKFTGLGIYDRILILPTHADP |
| | | | GSEIQVNGIHTKVLDSYDLFN |
| | | | KEGVKLEITVLKLDRNEKFR |
| | | | DIRKYIPESEDDYPECNLALV |
| | | | ANQTEPTIIKVGDVVSYGNIL |
| | | | LSGTQTARMLKYNYPTKSGY |
| | | | CGGVLYKIGQILGIHVGGNGR |
| | | | DGFSSMLLRSYFTEQ |
| | | | (SEQ ID NO: 8) |
| M protein | Vesicular | Rhabdoviridae | MSSLKKILGLKGKGKKSKKL |
| | stomatitis virus | | GIAPPPYEEDTSMEYAPSAPID |
| | | | KSYFGVDEMDTYDPNQLRYE |
| | | | KFFFTVKMTVRSNRPFRTYSD |
| | | | VAAAVSHWDHMYIGMAGKR |
| | | | PFYKILAFLGSSNLKATPAVL |
| | | | ADQGQPEYHTHCEGRAYLPH |
| | | | RMGKTPPMLNVPEHFRRPFNI |
| | | | GLYKGTIELTMTIYDDESLEA |
| | | | APMIWDHFNSSKFSDFREKA |
| | | | LMFGLIVEKKASGAWVLDSIS |
| | | | HFK |
| | | | (SEQ ID NO: 9) |
| Non-structural | Influenza A | Ortho- | MDPNTVSSFQVDCFLWHVRK |
| Protein 1 | virus | myxoviridae | RVADQELGDAPFLDRLRRDQ |
| | | | KSLRGRGSTLGLDIETATRAG |
| | | | KQIVERILKEESDEALKMTM |
| | | | ASVPASRYLTDMTLEEMSRD |
| | | | WSMLIPKQKVAGPLCIRMDQ |
| | | | AIMDKNIILKANFSVIFDRLET |
| | | | LILLRAFTEEGAIVGEISPLPSL |
| | | | PGHTAEDVKNAVGVLIGGLE |
| | | | WNDNTVRVSETLQRFAWRSS |
| | | | NENGRPPLTPKQKREMAGTI |
| | | | RSEV |
| | | | (SEQ ID NO: 10) |
| Immediate- | Simplexvirus | Herpesviridae | MATDIDMLIDLGLDLSDSDL |
| early protein | | | DEDPPEPAESRRDDLESDSSG |
| IE63 | | | ECSSSDEDMEDPHGEDGPEPI |
| | | | LDAARPAVRPSRPEDPGVPST |
| | | | QTPRPTERQGPNDPQPAPHSV |
| | | | WSRLGARRPSCSPEQHGGKV |
| | | | ARLQPPPTKAQPARGGRRGR |
| | | | RRGRGRGGPGAADGLSDPRR |
| | | | RAPRTNRNPGGPRPGAGWTD |
| | | | GPGAPHGEAWRGSEQPDPPG |
| | | | GQRTRGVRQAPPPLMTLAIAP |
| | | | PPADPRAPAPERKAPAADTID |
| | | | ATTRLVLRSISERAAVDRISES |
| | | | FGRSAQVMHDPFGGQPFPAA |
| | | | NSPWAPVLAGQGGPFDAETR |
| | | | RVSWETLVAHGPSLYRTFAG |
| | | | NPRAASTAKAMRDCVLRQE |
| | | | NFIEALASADETLAWCKMCI |
| | | | HHNLPLRPQDPIIGTTAAVLD |
| | | | NLATRLRPFLQCYLKARGLC |
| | | | GLDELCSRRRLADIKDIASFV |
| | | | FVILARLANRVERGVAEIDYA |
| | | | TLGVGVGEKMHFYLPGACM |
| | | | AGLIEILDTHRQECSSRVCELT |
| | | | ASHIVAPPYVHGKYFYCNSLF |
| | | | (SEQ ID NO: 11) |

Fusion Proteins

In some embodiments, the target protein and the enhancer protein are comprised in a single fusion protein. In some embodiments, the fusion protein may comprise a linking element. In some embodiments, the linking element may comprise a cleavage site for enzymatic cleavage. In other embodiments, the fusion protein or the linking element does not comprise a cleavage site and the expressed fusion protein comprises both the target protein and the enhancer protein.

Protein Modifications

The target proteins, enhancer proteins, and/or fusion proteins, or the polynucleotides encoding such, may be modified to comprise one or more markers, labels, or tags. For example, in some embodiments, a protein of the present disclosure may be labeled with any label that will allow its detection, e.g., a radiolabel, a fluorescent agent, biotin, a peptide tag, an enzyme fragment, or the like. The proteins may comprise an affinity tag, e.g., a His-tag, a FLAG tag, a GST-tag, a Strep-tag, a biotin-tag, an immunoglobulin binding domain, e.g., an IgG binding domain, a calmodulin binding peptide, and the like. In some embodiments, the FLAG tag comprises the amino acid sequence DYKDDDDK (SEQ ID NO: 21). In some embodiments, polynucleotides of the present disclosure comprise a selectable marker, e.g., an antibiotic resistance marker.

Polymerases

For the transcription of the polynucleotides encoding the target protein(s) and enhancer protein(s), an endogenous or exogenous polymerase may be used. In some embodiments, transcription of the polynucleotide(s) is performed by the natural polymerases comprised by the cell (e.g., eukaryotic cell). Viral polymerases may alternatively or additionally be used. In some embodiments, a viral promoter is used in combination with one or more viral polymerase. In some embodiments, eukaryotic promoters are used in combination with one or more eukaryotic polymerases. Illustrative viral polymerases include, but are not limited to, T7, T5, EMCV, HIV, Influenza, SP6, CMV, T3, T1, SP01, SP2, Phi 15, and the like. Viral polymerases are RNA priming or capping polymerases. In some embodiments, IRES elements are used in conjunction with viral polymerases.

A vector or vectors according to the present disclosure may comprise a polynucleotide sequence encoding a polymerase. In some embodiments, the polymerase is a viral polymerase. The polynucleotide sequence encoding the polymerase may be comprised by a vector that comprises a target protein-encoding polynucleotide and/or an enhancer protein-encoding polynucleotide. In some embodiments, the polymerase may be comprised by a vector that does not comprise target protein or enhancer protein-encoding polynucleotides.

In some embodiments, at least one of the one or more vectors comprised by the systems, methods, or cells disclosed herein may comprise a polynucleotide sequence encoding a T7 RNA polymerase.

Vectors

In some aspects, the present disclosure relates to vectors comprising nucleic acid sequences for the expression of one or more target proteins and one or more enhancer proteins. In some embodiments, the vectors (or a vector) have a first polynucleotide encoding the target protein and a second polynucleotide encoding an enhancer protein. In some embodiments, the vectors (or a vector) comprises any one of the expression cassettes disclosed herein, for instance, an adeno-associated virus (AAV) expression cassette, which comprises a 5′ inverted terminal repeat (ITR), any one of the nucleic acid sequences disclosed herein for the expression of one or more target proteins and one or more enhancer proteins, and a 3′ ITR, and/or nucleic acid sequences encoding AAV capsid proteins.

A vector for use according to the present disclosure may comprise any vector known in the art. In certain embodiments, the vector is any recombinant vector capable of expression of a protein or polypeptide of interest or a fragment thereof, for example, an adeno-associated virus (AAV) vector, a lentivirus vector, a retrovirus vector, a replication competent adenovirus vector, a replication deficient adenovirus vector, a herpes virus vector, a baculovirus vector or a non-viral plasmid. In some embodiments, the vector is a viral vector, a plasmid, a phage, a phagemid, a cosmid, a fosmid, a bacteriophage or an artificial chromosome. In some embodiments, the vector is a viral vector comprising an adenovirus vector, a retroviral vector or an adeno-associated viral vector. In some embodiments, the vector is a bacterial artificial chromosome (BAC), a plasmid, a bacteriophage P1-derived vector (PAC), a yeast artificial chromosome (YAC), or a mammalian artificial chromosome (MAC).

Cells, systems, and methods disclosed herein may comprise one vector. In some embodiments, the cells, systems, and methods may comprise a single vector comprising a first polynucleotide encoding a target protein and a second polynucleotide encoding an enhancer protein.

Cells, systems, and methods disclosed herein may comprise two vectors. In some embodiments, the cells, systems, and methods may comprise a first vector comprising the first polynucleotide, operatively linked to a first promoter; and a second vector comprising the second polynucleotide, operatively linked to a second promoter.

Cells, systems, and methods disclosed herein may comprise more than two vectors, wherein the vectors may encode target protein(s) and enhancer protein(s) in a variety of combinations or configurations.

In some embodiments, provided is a cell comprising a vector or vectors of the disclosure. In some embodiments, provided is a cell comprising polynucleotides of the disclosure. In some embodiments, provided is a cell expressing target protein(s) and enhancer protein(s) of the disclosure.

Promoters

Vectors according to the present disclosure may comprise one or more promoters. The term “promoter” refers to a region or sequence located upstream or downstream from the start of transcription which is involved in recognition and binding of RNA polymerase and other proteins to initiate transcription. The polynucleotide(s) or vector(s) according to the present disclosure may comprise one or more promoters. The promoters may be any promoter known in the art. The promoter may be a forward promoter or a reverse promoter. In some embodiments, the promoter is a mammalian promoter. In some embodiments, one or more promoters are native promoters. In some embodiments, one or more promoters are non-native promoters. In some embodiments, one or more promoters are non-mammalian promoters. Non-limiting examples of RNA promoters for use in the disclosed compositions and methods include U1, human elongation factor-1 alpha (EF-1 alpha), cytomegalovirus (CMV), human ubiquitin, spleen focus-forming virus (SFFV), U6, H1, tRNA^{Lys}, tRNA^{Ser} and tRNA^{Arg}, CAG, PGK, TRE, UAS, UbC, SV40, T7, Sp6, lac, araBad, trp, and Ptac promoters.

The term “operatively linked” as used herein refers to elements or structures in a nucleic acid sequence that are linked by operative ability and not physical location. The elements or structures are capable of, or characterized by, accomplishing a desired operation. It is recognized by one of ordinary skill in the art that it is not necessary for elements or structures in a nucleic acid sequence to be in a tandem or adjacent order to be operatively linked.

In some embodiments, the promoter drives the expression of one or more target proteins and/or one or more enhancer proteins constitutively; that is, the promoter is a constitutive promoter. In some embodiments, the promoter is an inducible promoter. The inducible promoter is not limited, and may be any inducible promoter known in the art. In some embodiments, the expression of the inducible promoter is promoted by the presence of one or more environmental or chemical stimuli. For instance, in some embodiments, the inducible promoter drives expression in the presence of a chemical molecule such as tetracycline and derivatives thereof (such as, doxycycline), cumate and derivatives thereof; or environmental stimuli, such as heat or light.

In some embodiments, the inducible promoter is based on the tetracycline-controlled transcriptional activation system, the cumate repressor system, the lac repressor system, arabinose-regulated pBad promoter system, alcohol-regulated AlcA promoter system, steroid-regulated LexA promoter system, heat shock inducible Hsp70 or Hsp90 promoter system, or blue light inducible pR promoter system. Thus, in some embodiments, the inducible promoter comprises a nucleic acid sequence that binds to a tetracycline transactivator, such as a tetracycline response element. In some embodiments, the expression of the inducible promoter is turned on in the presence of tetracycline and derivatives thereof (Tet-On system), while in other embodiments, the expression of the inducible promoter is turned off in the presence of tetracycline and derivatives thereof (Tet-Off system). In some embodiments, the inducible promoter is based on the cumate repressor system. Thus, in some embodiments, the inducible promoter comprises a nucleic acid sequence that binds to a CymR repressor, such as a cumate operator sequence.

In some embodiments, the expression of the inducible promoter is driven by the dimerization of a transcription factor. In some embodiments, the transcription is bacterial EL222, which dimerizes in the presence of blue light to drive expression from C120 promoter or a regulatory element thereof. In some embodiments, the inducible promoter comprises a nucleic acid sequence derived from the C120 promoter or regulatory element.

A vector according to the present disclosure may comprise one or more viral promoters that enable transcription of one or more polynucleotides by one or more viral polymerases. In some embodiments, for example, a vector may comprise a T7 promoter configured for transcription of either or both of the first polynucleotide (i.e., the target protein-encoding polynucleotide) or the second polynucleotide (i.e., the enhancer protein-encoding polynucleotide) by a T7 RNA polymerase.

Expression Cassettes

A vector or vectors according to the present disclosure may comprise one or more expression cassettes. The phrase “expression cassette” as used herein refers to a defined segment of a nucleic acid molecule that comprises the minimum elements needed for production of another nucleic acid or protein encoded by that nucleic acid molecule. In some embodiments, a vector may comprise an expression cassette, the expression cassette comprising a first polynucleotide encoding a target protein and a second polynucleotide encoding an enhancer protein. In some embodiments, the expression cassette comprises a first promoter, operatively linked to the first polynucleotide; and a second promoter, operatively linked to the second polynucleotide. In some embodiments, the expression cassette comprises a shared promoter operatively linked to both the first polynucleotide and the second polynucleotide.

In some embodiments, the expression cassette comprises a coding polynucleotide comprising the first polynucleotide and the second polynucleotide linked by a polynucleotide encoding a separating element (e.g., a ribosome skipping site or 2A element), the coding polynucleotide operatively linked to the shared promoter.

In some embodiments, the expression cassette comprises a coding polynucleotide, the coding polynucleotide encoding the enhancer protein and the target protein linked to by a separating element (e.g., a ribosome skipping site or 2A element), the coding polynucleotide operatively linked to the shared promoter.

In some embodiments, the expression cassette is configured for transcription of a single messenger RNA encoding both the target protein and the enhancer protein, linked by a separating element (e.g., a ribosome skipping site or 2A element); wherein translation of the messenger RNA results in expression of the target protein and the enhancer protein (e.g., the L protein) as distinct polypeptides.

In some embodiments, the expression cassette comprises a coding polynucleotide, the coding polynucleotide encoding the enhancer protein and the target protein as a fusion protein with or without a polypeptide linker, optionally wherein the polypeptide linker is a cleavable linker.

In some embodiments, the expression cassette is an adeno-associated virus (AAV) expression cassette, which comprises a 5′ inverted terminal repeat (ITR), any one of the nucleic acid sequences disclosed herein for the expression of one or more target proteins and one or more enhancer proteins, and a 3′ ITR. In some embodiments, the AAV expression cassette comprises a Kozak sequence, a polyadenylation sequence, and/or a stuffer sequence.

Separating Elements

In some embodiments, target protein(s) and enhancer protein(s) according to the present disclosure are encoded on the same vector or are encoded on separate vectors. In some embodiments, if nucleic acid sequences for one or more target proteins and one or more enhancer proteins are comprised by the same vector, the vector may comprise a separating element for separate expression of the proteins. In various embodiments, the vector is a bicistronic vector or a polycistronic vector. The separating element may be an internal ribosomal entry site (IRES) or 2A element. In some embodiments, a vector may comprise a nucleic acid encoding a 2A self-cleaving peptide. Illustrative 2A self-cleaving peptides include P2A, E2A, F2A, and T2A.

In some embodiments, the first polynucleotide or the second polynucleotide, or both, are operatively linked to an internal ribosome entry site (IRES).

In some embodiments, the first polynucleotide or the second polynucleotide, or both, are operatively linked to a 2A element.

Recombinant AAV Particles

The disclosure provides a recombinant viral vector comprising any one of the expression cassettes disclosed herein. In some embodiments, the viral vector is an adeno-associated virus (AAV) vector, a lentivirus vector, a retrovirus vector, a replication competent adenovirus vector, a replication deficient adenovirus vector, a herpes virus vector, or a baculovirus vector.

The disclosure provides methods for producing a recombinant AAV (rAAV) vector, comprising contacting an adeno-associated virus (AAV) producer cell (e.g., an HEK293 cell) with any one of the AAV expression cassettes disclosed herein, or a vector (e.g., plasmid or bacmid) comprising any one of the AAV expression cassettes disclosed herein. In some embodiments, the vectors (e.g., plasmid or bacmid) disclosed herein further comprise one or more genetic elements used during production of AAV, including, for example, AAV rep and cap genes, and/or encode helper virus protein sequences.

In some embodiments, the method comprises contacting the AAV producer cell with one or more additional plasmids comprising, for example, AAV rep and cap genes, and/or encoding helper virus protein sequences. In some embodiments, the method further comprises maintaining the AAV producer cell under conditions such that AAV is produced.

The disclosure provides rAAV vectors produced using any one of the methods disclosed herein. The rAAV vectors produced may be of any serotype, for example AAV1, AAV2, AAV3, AAV4, AAVS, AAV6, AAV7, AAV8, AAV9, AAV10, AAV11, AAV12, AAVrh8, AAVrh10, AAVrh32.33, AAVrh74, Avian AAV or Bovine AAV. In some embodiments, the recombinant AAV vectors produced may comprise one or more amino acid modifications (e.g., substitutions and/or deletions) compared to the native AAV capsid. In some embodiments, the recombinant AAV vector is a single-stranded AAV (ssAAV). In some embodiments, the recombinant AAV vector is a self-complementary AAV (scAAV).

The disclosure further provides compositions, such as a pharmaceutical composition, comprising any one of the expression cassettes, any one of the vectors (such as, any one of the recombinant AAV vectors), or any one of the AAV producer cells disclosed herein. In some embodiments, the pharmaceutical composition comprises one or more pharmaceutically acceptable carriers.

The disclosure further provides a vaccine composition, comprising any one of the expression cassettes, any one of the vectors (such as, any one of the recombinant AAV vectors), or any one of the AAV producer cells disclosed herein, wherein the target protein is a protein that upon expression in a subject, can elicit an immune response against a pathogen in the subject, or be of other therapeutic nature.

In some embodiments, the target protein is derived from the pathogen. The pathogen may be a virus, a bacteria, a fungus, or a parasite. In some embodiments, the virus is selected from the group consisting of SARS-CoV-2, SARS-CoV-1, MERS-CoV, chikungunya virus, African Swine Fever virus, Dengue virus, Zika virus, Influenza virus (e.g., A, B, C), Human Immunodeficiency Virus (HIV), Ebola virus, Hepatitis virus (e.g., Hepatitis A, Hepatitis B, Hepatitis C, Hepatitis D, and Hepatitis E), herpes simplex virus type 1 (HSV-1), herpes simplex virus type 2 (HSV-2) and Human Papillomavirus. In some embodiments, the pathogenic parasite is Plasmodium falciparum, Plasmodium vivax, Plasmodium malariae, Plasmodium ovale, Entamoeba histolytica, Leishmania donovani, Trypanosoma brucei, Giardia lamblia. In some embodiments, the pathogenic bacteria is selected from the group consisting of Bacillus subtilis, Clostridium botulinum, Corynebacterium diphtheria, Enterococcus faecalis, Escherichia coli, Francisella tularensis, Haemophilus influenzae, Helicobacter pylori, Listeria monocytogenes, Mycobacterium tuberculosis, Mycobacterium leprae, Pseudomonas aeruginosa, Rickettsia rickettsia, Salmonella typhi, Staphylococcus aureus, Streptococcus pneumonia, and Vibrio cholera. In some embodiments, the vaccine composition comprises one or more adjuvants.

Transfection, Transduction, Transformation

The terms “transfection,” “transduction,” and “transformation” refer to the process of introducing nucleic acids into cells (e.g., eukaryotic cells). A polynucleotide or vector described herein can be introduced into a cell (e.g., a eukaryotic cell) using any method known in the art. A polynucleotide or vector may be introduced into a cell by a variety of methods, which are well known in the art and selected, in part, based on the particular host cell. For example, the polynucleotide can be introduced into a cell using chemical, physical, biological, or viral means. Methods of introducing a polynucleotide or a vector into a cell include, but are not limited to, the use of calcium phosphate, dendrimers, cationic polymers, lipofection, fugene, peptide dendrimers, electroporation, cell squeezing, sonoporation, optical transfection, protoplast fusion, impalefection, hydrodynamic delivery, gene gun, magnetofection, particle bombardment, nucleofection, and viral transduction.

Vectors comprising targeting DNA and/or nucleic acid encoding a target protein and an enhancer protein can be introduced into a cell by a variety of methods (e.g., injection, transformation, transfection, direct uptake, projectile bombardment, liposomes). Target proteins and enhancer proteins can be stably or transiently expressed in cells using expression vectors. Techniques of expression in eukaryotic cells are well known to those in the art. (See Current Protocols in Human Genetics: Chapter 12 “Vector Therapy” & Chapter 13 “Delivery Systems for Gene Therapy”).

In some embodiments, polynucleotides or vectors can be introduced into a host cell by insertion into the genome using standard methods to produce stable cell lines, optionally through the use of lentiviral transfection, baculovirus gene transfer into mammalian cells (BacMam), retroviral transfection, CRISPR/Cas9, and/or transposons. In some embodiments, polynucleotides or vectors can be introduced into a host cell for transient transfection. In some embodiments, transient transfection may be effected through the use of viral vectors, helper lipids, e.g., PEI, Lipofectamine, and/or Fectamine 293. The genetic elements can be encoded as DNA on e.g. a vector or as RNA from e.g. PCR. The genetic elements can be separated in different or combined on the same vector.

Cells, Cell Lines, Host Cells

Another aspect of the present disclosure relates to cells comprising polynucleotides and/or vectors encoding one or more target proteins and one or more enhancer proteins. The polynucleotides, vectors, target protein, and enhancer proteins may be any of those described herein. The disclosure further provides cells or cell lines comprising polynucleotides and/or vectors encoding one or more enhancer proteins; these cells or cell lines may be referred to herein as “super-producer cells” or “super-producer cell lines”. In some embodiments, super-producer cells further comprise polynucleotides and/or vectors encoding one or more target proteins. Without being bound by any one theory, it is thought that cells expressing one or more enhancer proteins as disclosed herein are capable of serving as host cells for the expression of one or more target proteins.

In some embodiments, the cell is any eukaryotic cell or cell line. The disclosed polynucleotides, vectors, systems, and methods may be used in any eukaryotic cell lines. Eukaryotic cell lines may include mammalian cell lines, such as human and animal cell lines. Eukaryotic cell lines may also include insect, plant, or fungal cell lines. Non-limiting examples of such cells or cell lines generated from such cells include Bc HROC277, COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, 5P2/0-Ag14, HeLa, HEK293 (e.g., HEK293-F, HEK293-H, HEK293-T), and perC6 cells as well as insect cells such as Spodoptera fugiperda (Sf, e.g., Sf9), or fungal cells such as Saccharomyces, Pichia and Schizosaccharomyces.

In some embodiments, a cell or cell line for expressing target protein(s) and enhancer protein(s) is a human cell or cell line. In certain aspects, the choice of a human cell line is beneficial, e.g., for post-translational modifications (“PTMs”), such as glycosylation, phosphorylation, disulfide bonds, in target proteins. In some embodiments, a human cell or cell line is used for expression of a human target protein.

In some embodiments, the cell line is a stable cell line. In some embodiments, the cell is transiently transfected with any one or more of the polynucleotides and/or vectors disclosed herein.

In some embodiments, the present disclosure provides a eukaryotic cell for expression of a target protein, wherein the cell comprises an exogenous polynucleotide encoding an enhancer protein. In some embodiments, the exogenous polynucleotide encoding an enhancer protein is transiently transduced and/or not integrated into the genome of the cell. In some embodiments, the exogenous polynucleotide encoding an enhancer protein is stably integrated. In some embodiments, the enhancer protein is an inhibitor of nucleocytoplasmic transport (NCT). In some embodiments, the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein. The exogenous polynucleotide is operatively linked to a promoter (optionally a native promoter or an exogenous promoter). In some embodiments, the polynucleotide is operatively linked to an internal ribosome entry site (IRES).

Methods of Protein Expression

The present disclosure provides a method for expressing a target protein in eukaryotic cells. The method may comprise introducing a polynucleotide encoding the target protein (the polynucleotide operatively linked to a promoter) into the eukaryotic cells. This method utilizes co-expression of an enhancer protein to enhance the expression level, solubility and/or activity of the target protein.

In some embodiments, the expression level of a target protein expressed in combination with one or more enhancers according to the methods of the disclosure is higher than the expression level of the target protein expressed in the absence of the one or more enhancers. In some embodiments, the expression level of the target protein expressed in combination with one or more enhancers according to the methods of the disclosure is at least about 1.1-fold (for example, about 1.2 fold, about 1.3 fold, about 1.4 fold, about 1.5 fold, about 1.6 fold, about 1.7 fold, about 1.8 fold, about 1.9 fold, about 2-fold, about 2.5-fold, about 3-fold, about 3.5-fold, about 4-fold, about 4.5-fold, about 5-fold, about 6-fold, about 7-fold, about 8-fold, about 9-fold, or about 10-fold) higher as compared to the expression level of the target protein expressed in the absence of the one or more enhancers.

In some embodiments, the activity of a target protein expressed in combination with one or more enhancers according to the methods of the disclosure is higher than the activity of the target protein expressed in the absence of the one or more enhancers. In some embodiments, the activity of the target protein expressed in combination with one or more enhancers according to the methods of the disclosure is at least about 1.1-fold (for example, about 1.2 fold, about 1.3 fold, about 1.4 fold, about 1.5 fold, about 1.6 fold, about 1.7 fold, about 1.8 fold, about 1.9 fold, about 2-fold, about 2.5-fold, about 3-fold, about 3.5-fold, about 4-fold, about 4.5-fold, about 5-fold, about 6-fold, about 7-fold, about 8-fold, about 9-fold, or about 10-fold) higher as compared to the activity of the target protein expressed in the absence of the one or more enhancers.

In some embodiments, the enhancer protein is an inhibitor of nucleocytoplasmic transport (NCT). In some embodiments, the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

In some aspects, the present disclosure relates to methods of producing target proteins through the use of cells comprising polynucleotides encoding one or more target proteins and one or more enhancer proteins. In some embodiments, the method is carried out in eukaryotic cells comprising one or more vectors. In some embodiments, the method is carried out using the polynucleotides, vectors, and cells described in the foregoing sections. In some embodiments, the vectors (or a vector) may have a first polynucleotide encoding the target protein and a second polynucleotide encoding an enhancer protein. In some embodiments, the first polynucleotide and the second polynucleotide are operatively linked to one or more promoters.

Further provided is a method for recombinant expression of a target protein that includes introducing a polynucleotide encoding the target protein, operatively linked to a promoter, into a eukaryotic cell. In some embodiments, the method of target protein expression comprises introducing a vector system of the disclosure into a eukaryotic cell. In some embodiments, the target protein is a membrane protein. In some embodiments, localization of the membrane protein to the cellular membrane is increased compared to the localization observed when the membrane protein is expressed without the enhancer protein. In some embodiments, the level of the membrane-associated membrane protein expressed in combination with one or more enhancers according to the methods of the disclosure is at least about 1.1-fold (for example, about 1.2 fold, about 1.3 fold, about 1.4 fold, about 1.5 fold, about 1.6 fold, about 1.7 fold, about 1.8 fold, about 1.9 fold, about 2-fold, about 2.5-fold, about 3-fold, about 3.5-fold, about 4-fold, about 4.5-fold, about 5-fold, about 6-fold, about 7-fold, about 8-fold, about 9-fold, or about 10-fold) higher, as compared to the level of the membrane-associated membrane protein expressed in the absence of the one or more enhancers.

In some embodiments, the expression of one or more enhancer proteins disclosed herein using the methods disclosed herein may be associated with, correlated with, or result in an effect on the cell cycle of the host cells, such that the number of enhancer-expressing host cells in a specific cell cycle stage is altered, as compared to wild type cells that do not express the one or more enhancer proteins. In some embodiments, the expression of one or more enhancer proteins disclosed herein using the methods disclosed herein may be associated with, correlated with, or result in the arrest of the host cell in a specific stage of the cell cycle. In some embodiments, the specific cell stage is the growth phase of the cell cycle, such as G1, S or G2 phase. In some embodiments, the expression of one or more enhancer proteins disclosed herein using the methods disclosed herein may be associated with, correlated with, or result in a reduction or elimination of clonal drift in the cells.

In some embodiments, the method may comprise introducing into a eukaryotic cell a polynucleotide encoding an enhancer protein, operatively linked to a promoter. In some embodiments, the method may comprise transfection of the eukaryotic cells with one or more DNA molecules, transduction of the eukaryotic cells with a single viral vector, and/or transduction of the eukaryotic cells with two or more viral vectors.

Downstream Applications

In some embodiments, target proteins, and cells expressing such proteins, produced through the use of the present compositions, systems, and methods are isolated, purified, and/or used for downstream applications. Illustrative applications include, but are not limited to, small molecule screening, structural determination (e.g., X-ray crystallography, cryo-electron microscopy, and the like), activity assays, therapeutics, enzyme replacement therapy, screening assays, diagnostic assays, clinical testing kits, drug discovery, antibody discovery, and the like. In some embodiments, the present compositions and methods are used to produce antibodies or to produce antigens for antibody screening assays. In some embodiments, the cells expressing the target proteins can be used as an assay system to screen, e.g., cell interactions, antibody binding, or small molecule influences in a whole cell system.

In some embodiments, the disclosure provides systems and methods for antibody discovery. In some embodiments, the disclosure provides methods for generating an antibody against a target protein, comprising immunizing a subject with a cell or target protein produced using the systems or methods of the disclosure. In various embodiments, the immunized subject is a mouse, rat, rabbit, non-human primate, lama, camel, or human. Cells isolated from the subject can be subjected to further rounds of the selection as isolated cells, or optionally after generation of hybridomas from the isolated cells. Gene cloning and/or sequencing can be used to isolate polynucleotide sequence(s) encoding heavy and light chains form the isolated cells or hybridomas. Gene cloning and/or sequencing can be applied to single cells or populations of cells. In some embodiments, the compositions and methods of the disclosure are used for generating a polyclonal antibody through immunization of a subject followed by harvesting of serum from the subject.

The disclosure further provides methods for antibody discovery by cell sorting, comprising providing a solution comprising a labeled cell or target protein produced using the systems or methods of the disclosure, and a population of recombinant cells, wherein the recombinant cells express a library of polypeptides each comprising an antibody or antigen-binding fragment thereof; and sorting one or more recombinant cells from the solution by detecting recombinant cells bound to the labeled cell or the labeled target protein. In other variations, cell sorting is performed on cells derived from an immunized subject. The subject may be immunized with a cell or target protein produced according the methods of the disclosure, or using another suitable immunogen. In some embodiments, the recombinant cells comprise a naive antibody library, optionally a human naive antibody library. Various antibody library generation methods are known in the art and can be combined with the methods of the present disclosure. As used herein, the terms “sorting” or “cell sorting” refer to fluorescence-activated cell sorting, magnetic assisted cell sorting, and other means of selecting labeled cells in a population of labeled and unlabeled cells.

The disclosure further provides, a method for panning a phage-display library, comprising mixing a phage-display library with a cell or target protein produced using the systems or methods of the disclosure; and purifying and/or enriching the members of the phage-display library that bind the cell or target protein. In some embodiments, the phage-display library expresses a population of single-chain variable fragments (scFvs) or other types of antibody/antibody fragments (Fabs etc.).

In further embodiments, the disclosure provides methods for screening for protein binders of any type. The cells and target proteins of the disclosure can be used to screen libraries of various types of molecule, including drugs and macromolecules (proteins, nucleic acids, and protein:nucleic acid complexes) to identify binding partners for the target protein. In other embodiments, the systems and methods of the disclosure are used to express libraries of target proteins in single wells, in pools of several sequences, or in libraries of gene sequences.

The ability to express an antigen in its native or disease-relevant form in high yields and/or present on the surface of cells enables more reliable discovery and/or generation of antibodies, antibody fragments, and other molecules than prior art methods. Such antibody, antibody fragments, and other molecules may be useful as therapeutics and/or research tools, or for other applications.

In some embodiments, the systems and methods of the disclosure are suitable for use in discovery of antibodies that bind to and/or are specific to particular glycosylation patterns on target molecules (e.g. glycoproteins). In some embodiments, the antibody library is sorted against the natively glycosylated protein and counter-sorted against an improperly glycosylated or de-glycosylated cognate protein. Similarly stated, by using a deglycosylation enzyme, antibodies can be sorted specifically against the glycosylation pattern. In further embodiments, the cells and/or target proteins of the disclosure are used to confirm the binding and/or functional activity of novel antibodies or other macromolecules.

In some embodiments, the systems and methods of the disclosure are suitable for use in the biosynthesis of any target protein in any host cell disclosed herein, or known in the art. For instance, the systems and methods of the disclosure are suitable for use in the biosynthesis of any target protein in mammalian cells, or using fermentation in bacteria, yeast and other microbes. In some embodiments, the systems and methods of the disclosure are suitable for use in the biosynthesis of non-protein molecules by the introduction of a specific metabolic pathway into the host cell. For instance, the non-protein molecule is an opioid molecule, or another metabolite.

Illustrative Advantages

The present compositions, systems, and methods may have numerous advantages. For example, as demonstrated in Example 11, a human NADase that usually results in apoptosis and therefore produces non-detectable yields when overexpressed in human cell lines, can be reliably expressed to produce yields of greater than 20 mg/L when an enhancer protein is co-expressed with this target protein. Additionally, the NADase expressed through this illustrative method is functional (as demonstrated by a phosphate release assay) and shows a low batch to batch variation.

Similarly, in some embodiments, the present methods, systems, and cells are used for the reliable expression of difficult to express proteins. In some embodiments, the present disclosure relates to the production of proteins with low batch-to-batch variation. The proteins produced according to the present disclosure may exhibit one or more of the following improvements: purification without purification tag fusions; improved functional activity; reliable production; consistent activity; and suitability for therapeutic applications.

Cells of the present disclosure may have one or more of the following advantages in terms of target protein expression: higher concentration of target membrane proteins in the membrane; slower/decreased target protein degradation; improved signal to noise ratio in whole cell assays; target protein and/or enhancer protein expression without affecting downstream cell metabolism; increased stability against desensitization of membrane-bound membrane proteins; and higher target protein yield. Example 1 provides an illustrative example of expression of enhancer protein without affecting downstream metabolism of cells. The GPCR exemplified in Example 1 was able to interact with its natural substrate and produce activation that could be measured in vitro.

The present systems and methods may, in some embodiments, have one or more of the following advantages: suitability for any eukaryotic cell type; decreased need for target protein expression optimization; and reliable expression of difficult-to-express proteins.

Systems

One aspect of the present disclosure provides a system for recombinant expression of a target protein in eukaryotic cells that includes one or more vectors. The vectors (or a vector) may have a first polynucleotide encoding a target protein and a second polynucleotide encoding an enhancer protein. The enhancer protein may be an inhibitor of nucleocytoplasmic transport (NCT). In some embodiments, the enhancer protein may be selected from the group consisting of a picornavirus leader (L) protein, a picornavirus 2A protease, a rhinovirus 3C protease, a herpes simplex virus (HSV) ICP27 protein, and a rhabdovirus matrix (M) protein. The first polynucleotide and the second polynucleotide may be operatively linked to one or more promoters.

In some embodiments, the enhancer protein is an inhibitor of nucleocytoplasmic transport (NCT). In some embodiments, the NCT inhibitor is a viral protein.

In some embodiments, the enhancer protein is an NCT inhibitor selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

The NCT inhibitor may be a picornavirus leader (L) protein or a functional variant thereof. In some embodiments, the NCT inhibitor may be a picornavirus 2A protease or a functional variant thereof. In some embodiments, the NCT inhibitor may be a rhinovirus 3C protease or a functional variant thereof. In some embodiments, the NCT inhibitor may be a coronavirus ORF6 protein or a functional variant thereof. In some embodiments, the NCT inhibitor may be an ebolavirus VP24 protein or a functional variant thereof. In some embodiments, the NCT inhibitor may be a Venezuelan equine encephalitis virus (VEEV) capsid protein or a functional variant thereof. In some embodiments, the NCT inhibitor is a herpes simplex virus (HSV) ICP27 protein or a functional variant thereof. In some embodiments, the NCT inhibitor is a rhabdovirus matrix (M) protein or a functional variant thereof.

In some embodiments, the enhancer protein is an L protein, which is the L protein of Theiler's virus or a functional variant thereof. In some embodiments, the L protein may share at least 90% identity to SEQ ID NO: 1.

In some embodiments, the L protein is the L protein of Encephalomyocarditis virus (EMCV) or a functional variant thereof. In some embodiments, the L protein may share at least 90% identity to SEQ ID NO: 2.

In some embodiments, the L protein is selected from the group consisting of the L protein of poliovirus, the L protein of HRV16, the L protein of mengo virus, and the L protein of Saffold virus 2 or a functional variant thereof.

The system may comprise a single vector comprising an expression cassette, the expression cassette comprising the first polynucleotide and the second polynucleotide. In some embodiments, the expression cassette comprises a first promoter, operatively linked to the first polynucleotide; and a second promoter, operatively linked to the second polynucleotide. In some embodiments, the expression cassette comprises a shared promoter operatively linked to both the first polynucleotide and the second polynucleotide.

In some embodiments, the expression cassette comprises a coding polynucleotide comprising the first polynucleotide and the second polynucleotide linked by a polynucleotide encoding a ribosome skipping site, the coding polynucleotide operatively linked to the shared promoter.

In some embodiments, the expression cassette comprises a coding polynucleotide, the coding polynucleotide encoding the enhancer protein and the target protein linked to by a ribosome skipping site, the coding polynucleotide operatively linked to the shared promoter.

In some embodiments, the expression cassette is configured for transcription of a single messenger RNA encoding both the target protein and the enhancer protein, linked by a ribosome skipping site; wherein translation of the messenger RNA results in expression of the target protein and the enhancer protein (e.g., an L protein) as distinct polypeptides.

The system may comprise one vector. In some embodiments, the system may comprise a single vector comprising a first polynucleotide encoding a target protein and a second polynucleotide encoding an enhancer protein.

The system may comprise two vectors. In some embodiments, the system may comprise a first vector comprising the first polynucleotide, operatively linked to a first promoter; and a second vector comprising the second polynucleotide, operatively linked to a second promoter.

In some embodiments, the first polynucleotide or the second polynucleotide, or both, are operatively linked to an internal ribosome entry site (IRES).

In some embodiments, at least one of the one or more vectors comprised by the system may comprise a T7 promoter configured for transcription of either or both of the first polynucleotide or the second polynucleotide by a T7 RNA polymerase.

In some embodiments, at least one of the one or more vectors comprised by the system may comprise a polynucleotide sequence encoding a T7 RNA polymerase.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

Unless the context indicates otherwise, it is specifically intended that the various features described herein can be used in any combination.

Unless otherwise defined, all technical and scientific terms used herein have the same meaning as commonly understood by one of ordinary skill in the art to which this disclosure belongs.

### EXAMPLES

TABLE 4
Table of Contents for Examples

| Exam- | Enhancer | Target | Cell | Type of |
|---|---|---|---|---|
| ples | Protein(s) | Protein | Line | target protein |
| 1 | ECMV L protein | GFP | HEK293 | Soluble Reporter |
| 2-4 | ECMV L protein | DRD1 | HEK293 | Membrane Protein |
| 5 | ECMV L protein | DRD1 | HEK293 | Membrane Protein |
| | Theiler's | | | |
| | virus L protein | | | |
| | Polio 2A protease | | | |
| | VSV M protein | | | |
| 6 | Theiler's | DRD1 | CHO-K1 | Membrane Protein |
| | virus L protein | | | |
| 7 | ECMV L protein | DRD1 | Sf9 | Membrane Protein |
| 8 | ECMV L protein | ITK | HEK293 | Kinase |
| 9 | ECMV L protein | ITK | CHO-K1 | Kinase |
| 10 | ECMV L protein | ITK | Sf9 | Kinase |
| 11 | ECMV L protein | CFTR | HEK293 | Membrane Protein |
| 12 | ECMV L protein | NADase | HEK293 | Hydrolase |
| 13 | ECMV L protein | C1-Inh | HEK293 | Secreted Protein |
| 14 | ECMV L protein | PSG1 | HEK293 | Secreted |
| | | | | Glycoprotein |

Materials and Methods

Construction of DNA Molecules

All assemblies were made into a plasmid backbone capable of propagation in E. coli comprising a promoter controlling a high copy number origin of replication (ColE1) followed by a terminator (rrnB T1 and T2 terminator). This is followed by a promoter controlling an antibiotic resistance gene which is isolated from the rest of the vector by a second terminator (transcription terminator from phage lambda). The genes comprising elements of the backbone were synthesized by phosphoramidite chemistry.

Structure genes used for the construction of the plasmids were synthesized by phosphoramidite chemistry, chemistry, amplified and cloned into the vector described above using an isothermal assembly reaction such as NEB HI-FI or Gibson Assembly using the primers listed in Table 2. Select amino acid sequences comprised by the illustrative constructs employed in these examples are provided in Table 3.

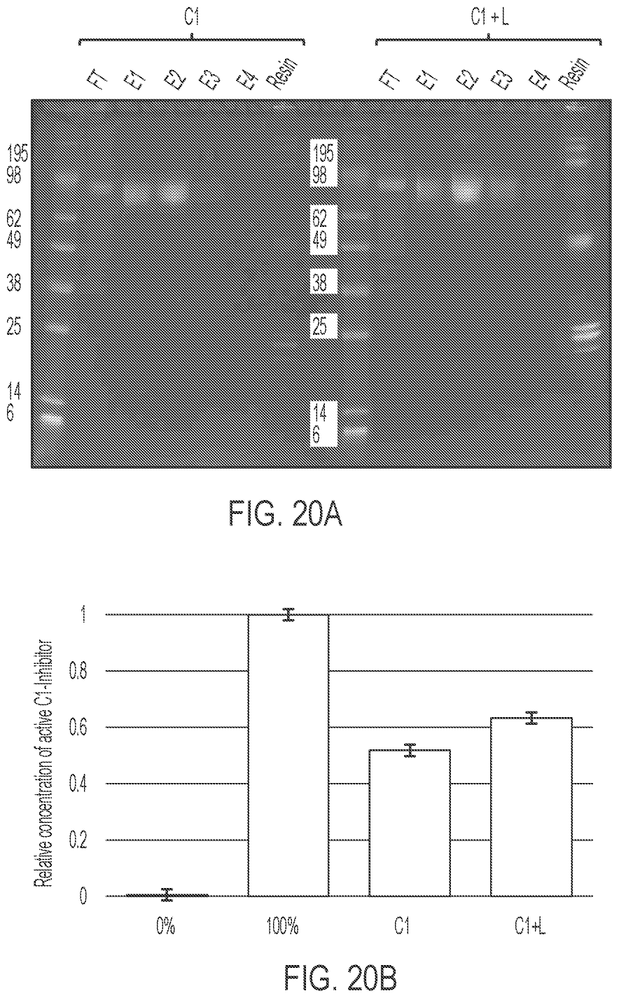

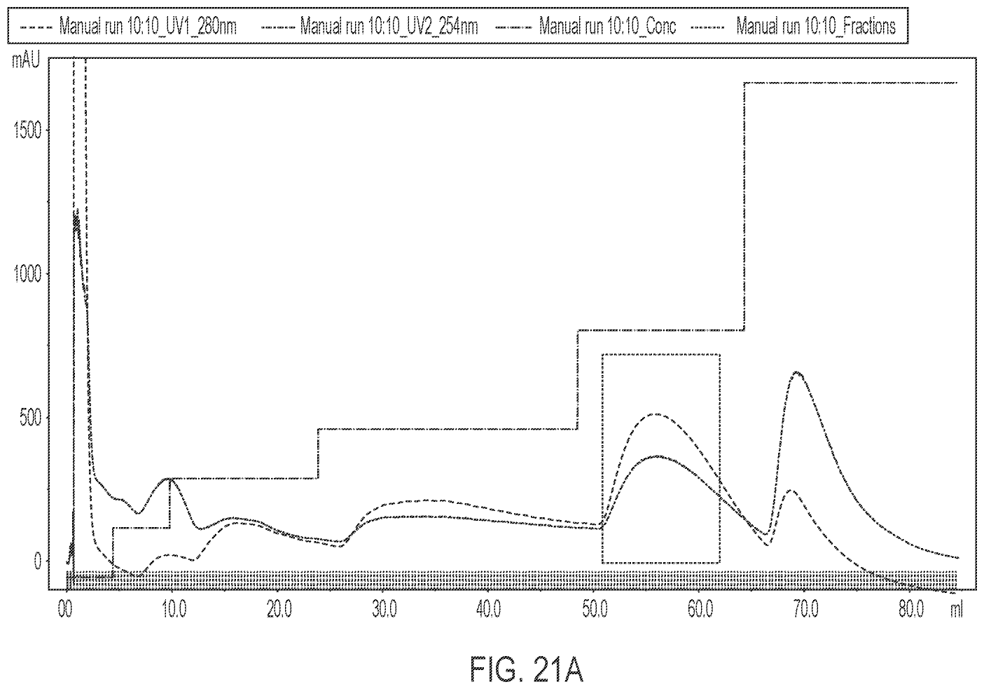

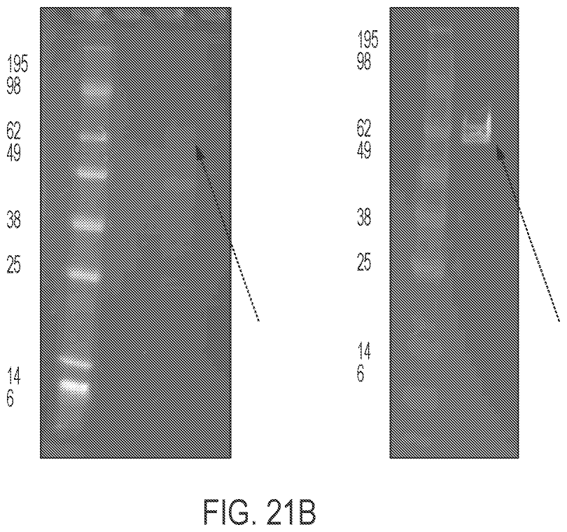

TABLE 2
Construct design

| | | | Used in |
|---|---|---|---|
| Construct | Schematic | Primer | Example |
| EG1 | FIG. 2A | 1a.) | 1 |
| | | gcccgggatccaccggtcgccaccatggtgagcaagggcgag | |
| | | gagc (SEQ ID NO: 22) | |
| | | 1b.) | |
| | | agatggctggcaactagaaggcacagttacttgtacagctcgtc | |
| | | catgccgag (SEQ ID NO: 23) | |
| | | 2a.) | |
| | | cactctcggcatggacgagctgtacaagtaactgtgccttctagtt | |
| | | gccagccatctgt (SEQ ID NO: 24) | |
| | | 2b.) | |
| | | cagctcctcgcccttgctcaccatggtggcgaccggtggatccc | |
| | | (SEQ ID NO: 25) | |
| EG2 | FIG. 2B | 1a.) | 2 |
| | | cggccagtaacgttaggggggggggattacttgtacagctcgtc | |
| | | catgccgag (SEQ ID NO: 26) | |
| | | 1b.) | |
| | | cggtaccgcgggcccgggatccaccggtcgccaccatggtga | |
| | | gcaagggcgaggagc (SEQ ID NO: 27) | |
| | | 2a.) | |
| | | cactctcggcatggacgagctgtacaagtaactgtgccttctagtt | |
| | | gccagccatctgt (SEQ ID NO: 24) | |
| | | 2b.) | |
| | | cagctcctcgcccttgctcaccatggtggcgaccggtggatccc | |
| | | (SEQ ID NO: 25) | |
| | | 3a.) | |
| | | ctcggcatggacgagctgtacaagtaatcccccccccctaacgt | |
| | | tactgg (SEQ ID NO: 28) | |
| | | 3b.) | |
| | | acgggggaggggcaaacaacagatggctggcaactagaagg | |
| | | cacagctgtaactcgaaaacgacttccatgtctaattcgg | |
| | | (SEQ ID NO: 29) | |
| EG3 | FIG. 2C | 1a.) | 1 |
| + | | cgcgggcccgggatccaccggtcgccaccATGAACAC | |
| EG4 | | CATCAATATTGCCAAGAACGACTTTTCT | |
| | | GACATCG (SEQ ID NO: 30) | |
| | | 1b.) | |
| | | agatggctggcaactagaaggcacagttagggTCAGGCA | |
| | | AATGCGAAATCGGACTCCAG (SEQ ID | |
| | | NO: 31) | |
| | | 2a.) | |
| | | CCTGGAGTCCGATTTCGCATTTGCCTGA | |
| | | ccctaactgtgccttctagttgccagccatctgt (SEQ ID | |
| | | NO: 32) | |
| | | 2b.) | |
| | | CGTTCTTGGCAATATTGATGGTGTTCAT | |
| | | ggtggcgaccggtggatcccgggcc (SEQ ID NO: 33) | |
| | | 3a) | |
| | | accttggccgactctggtaatgGTAATACGACTCAC | |
| | | TATAGGaaaaa (SEQ ID NO: 34) | |
| | | 3b. | |
| | | agtcagtgagcgaggaagccCAAAAAACCCCTCA | |
| | | AGACCCGTTTA (SEQ ID NO: 35) | |
| | | 4a) | |
| | | AAACGGGTCTTGAGGGGTTTTTTGggcttc | |
| | | ctcgctcactgac (SEQ ID NO: 36) | |
| | | 4b) | |
| | | TAGTGAGTCGTATTACcattaccagagtcggccaa | |
| | | ggt (SEQ ID NO: 37) | |
| EG5 | FIG. 2D | 1a) | 2 |
| | | gcccgggatccaccggtcgccacctcgccaccatgaggactct | |
| | | gaacacctctgccatgg (SEQ ID NO: 38) | |
| | | 1b) | |
| | | CTTTTCGAACTGCGGGTGGCTCCAGAG | |
| | | CGGCCGCGTtccCGTggttgggtgctgaccgttttgtgt | |
| | | g (SEQ ID NO: 39) | |
| | | 2a) | |
| | | ACGCGGCCGCTCTGGAGCCACCCGCAG | |
| | | TTCGAAAAGtaaagcggccgcgactctagatca | |
| | | (SEQ ID NO: 40) | |
| | | 2b) gtgttcagagtcctcatggtggcgaggtggcgacc | |
| | | (SEQ ID NO: 41) | |
| EG6 | FIG. 2E | 1a.) | 2 |
| | | atccaccggtcgccaccatgaggactctgaacacctctgccatg | |
| | | g (SEQ ID NO: 42) | |
| | | 1b.) | |
| | | tgtggtatggctgattatgatttactgtaactcgaaaacgacttcca | |
| | | tgtctaattcggg (SEQ ID NO: 43) | |
| | | 2a.) | |
| | | gttttcgagttacagtaaatcataatcagccataccacatttgtaga | |
| | | ggttttacttgct (SEQ ID NO: 44) | |
| | | 2b.) | |
| | | tggcagaggtgttcagagtcctcatggtggcgaccggtgg | |
| | | (SEQ ID NO: 45) | |
| EG7 | FIG. 2F | | 2 |
| EG8 | FIG. 2G | 1a.) | 2, 5 |
| | | atccaccggtcgccaccatgaggactctgaacacctctgccatg | |
| | | g (SEQ ID NO: 42) | |
| | | 1b.) | |
| | | cggccagtaacgttaggggggggggattacttgtacagctcgtc | |
| | | catgccgag (SEQ ID NO: 26) | |
| | | 2a.) | |
| | | ctcggcatggacgagctgtacaagtaatcccccccccctaacgt | |
| | | tactgg (SEQ ID NO: 28) | |
| | | 2b.) | |
| | | tggcagaggtgttcagagtcctcatggtggcgaccggtgg | |
| | | (SEQ ID NO: 45) | |
| EG9 | FIG. 2H | 1a) | 8 |
| | | CACCATCACCATCACCATGTTatggccacaac | |
| | | catggaacaagagactt (SEQ ID NO: 46) | |
| | | 1b) | |
| | | tcttgatgagctgttcttccaggaggataaagttgttcatggtggc | |
| | | gaccggtggatccc (SEQ ID NO: 47) | |
| | | 2a) | |
| | | cgggcccgggatccaccggtcgccaccatgaacaactttatcct | |
| | | cctggaagaacagctc (SEQ ID NO: 48) | |
| | | 2b) | |
| | | aagtctcttgttccatggttgtggccatAACATGGTGAT | |
| | | GGTGATGGTG (SEQ ID NO: 49) | |
| EG10 | FIG. 21 | 1a.) gtgttcagagtcctcatggtggcgaggtggcgacc | 2 |
| + | | (SEQ ID NO: 41) | |
| EG11 | | 1b.) | |
| | | CTCTCGGCATGGACGAGCTGTACAAG | |
| | | (SEQ ID NO: 50) | |
| | | 2a.) | |
| | | ttaCTTGTACAGCTCGTCCATGCCGAGAG | |
| | | (SEQ ID NO: 51) | |
| | | 2b.) | |
| | | gcccgggatccaccggtcgccacctcgccaccatgaggactct | |
| | | gaacacctctgccatgg (SEQ ID NO: 38) | |
| | | 3a) | |
| | | tgcgcgcaagtctcttgttccatggttgtggccatggtggcgacc | |
| | | ggtggatccc (SEQ ID NO: 52) | |
| | | 3b) cccgaattagacatggaagtcgttttcgagttacag | |
| | | (SEQ ID NO: 53) | |
| | | 4a) | |
| | | gggatccaccggtcgccaccatggccacaaccatggaacaag | |
| | | agacttg (SEQ ID NO: 54) | |
| | | 4b) ctgtaactcgaaaacgacttccatgtctaattcggg | |
| | | (SEQ ID NO: 55) | |
| EG12 | FIG. 2J | 1a) tctcttgttccatggttgtggccatggtggcgaccggtgg | 3 |
| + | | (SEQ ID NO: 56) | |
| EG4 | | 1b) | |
| | | acgtggttttcctttgaaaaacacgatgataaatgaggactctgaa | |
| | | cacctctgccatgg (SEQ ID NO: 57) | |
| | | 2a) | |
| | | gcagaggtgttcagagtcctcatttatcatcgtgtttttcaaaggaa | |
| | | aaccacg (SEQ ID NO: 58) | |
| | | 2b) | |
| | | agtcgttttcgagttacagtaatcccccccccctaacgttactgg | |
| | | (SEQ ID NO: 59) | |
| | | 3a) | |
| | | ccagtaacgttaggggggggggattactgtaactcgaaaacga | |
| | | cttccatgt (SEQ ID NO: 60) | |
| | | 3b) ccaccggtcgccaccatggccacaaccatggaacaagag | |
| | | (SEQ ID NO: 61) | |
| EG10 | FIG. 2K | 1a.) gtgttcagagtcctcatggtggcgaggtggcgacc | 2, 3, 4 |
| | | (SEQ ID NO: 41) | |
| | | 1b.) | |
| | | CTCTCGGCATGGACGAGCTGTACAAG | |
| | | (SEQ ID NO: 50) | |
| | | 2a.) | |
| | | ttaCTTGTACAGCTCGTCCATGCCGAGAG | |
| | | (SEQ ID NO: 51) | |
| | | 2b.) | |
| | | gcccgggatccaccggtcgccacctcgccaccatgaggactct | |
| | | gaacacctctgccatgg (SEQ ID NO: 38) | |
| EG13 | FIG. 2L | 1a.) | 11 |
| | | cgggcccgggatccaccggtcgccaccatgaacaactttatcct | |
| | | cctggaagaacagctc (SEQ ID NO: 48) | |
| | | 1b.) | |
| | | GATGGTGTCCCCCGCCACCTCCGCCACC | |
| | | TCCaagtcctgattctgcaatttcagccagtt (SEQ ID | |
| | | NO: 62) | |
| | | 2a.) | |
| | | aattgcagaatcaggacttGGAGGTGGCGGAGGT | |
| | | GGCGGGGGACACCATCACCATCACCAT | |
| | | GTTTAAtcccccccccctaacgttactgg (SEQ ID | |
| | | NO: 63) | |
| | | 2b.) | |
| | | tcttgatgagctgttcttccaggaggataaagttgttcatggtggc | |
| | | gaccggtggatccc (SEQ ID NO: 47) | |
| EG14 | FIG. 2M | 1a.) | 11 |
| | | ttataggcggacagcagcagggtcagcaccatggtggcgaggt | |
| | | ggcgacc (SEQ ID NO: 64) | |
| | | 1b.) | |
| | | CGGCCGCTCGATTACAAGGATGACGAC | |
| | | GATAAGGTTTAAagcggccgcgactctagatca | |
| | | (SEQ ID NO: 65) | |
| | | 2a.) | |
| | | TAAACCTTATCGTCGTCATCCTTGTAAT | |
| | | CGAGCGGCCGCGTtgtagggcccatgggggcg | |
| | | (SEQ ID NO: 66) | |
| | | 2b.) | |
| | | gcgggcccgggatccaccggtcgccacctcgccaccatggtg | |
| | | ctgaccctgctgctgtcc (SEQ ID NO: 67) | |
| EG15 | FIG. 2N | 1a) | 7 |
| | | ccctgtcttcatggggcgagtatatgaccccagggccGGAG | |
| | | GTGGCGGAGGTGGC (SEQ ID NO: 68) | |
| | | 1b) | |
| | | ggagggtcagcagggtcagcctggaggccatggtggcgaccg | |
| | | gtggatcc (SEQ ID NO: 69) | |
| | | 2a) | |
| | | cggtaccgcgggcccgggatccaccggtcgccaccatggcct | |
| | | ccaggctgaccctg (SEQ ID NO: 70) | |
| | | 2b) | |
| | | TGGTGTCCCCCGCCACCTCCGCCACCTC | |
| | | Cggccctggggtcatatactcgcc (SEQ ID NO: 71) | |
| EG16 | FIG. 20 | 1a) | 7 |
| | | ccctgtcttcatggggcgagtatatgaccccagggccGGAG | |
| | | GTGGCGGAGGTGGC (SEQ ID NO: 68) | |
| | | 1b) | |
| | | ggagggtcagcagggtcagcctggaggccatggtggcgaccg | |
| | | gtggatcc (SEQ ID NO: 69) | |
| | | 2a) | |
| | | cggtaccgcgggcccgggatccaccggtcgccaccatggcct | |
| | | ccaggctgaccctg (SEQ ID NO: 70) | |
| | | 2b) | |
| | | TGGTGTCCCCCGCCACCTCCGCCACCTC | |
| | | Cggccctggggtcatatactcgcc (SEQ ID NO: 71) | |
| EG17 | FIG. 2P | 1a.) | 8, 9, 10 |
| | | cgggcccgggatccaccggtcgccaccatgaacaactttatcct | |
| | | cctggaagaacagctc (SEQ ID NO: 48) | |
| | | 1b.) | |
| | | GATGGTGTCCCCCGCCACCTCCGCCACC | |
| | | TCCaagtcctgattctgcaatttcagccagtt (SEQ ID | |
| | | NO: 62) | |
| | | 2a.) | |
| | | tcttgatgagctgttcttccaggaggataaagttgttcatggtggc | |
| | | gaccggtggatccc (SEQ ID NO: 47) | |
| | | 2b.) | |
| | | aattgcagaatcaggacttGGAGGTGGCGGAGGT | |
| | | GGCGGGGGACACCATCACCATCACCAT | |
| | | GTTTAAtcccccccccctaacgttactgg (SEQ ID | |
| | | NO: 63) | |
| EG18 | FIG. 2Q | 1a) | 5 |
| | | aattgcagaatcaggacttGGAGGTGGCGGAGGT | |
| | | GGCGGGGGACACC (SEQ ID NO: 72) | |
| | | 1b) | |
| | | tcttgatgagctgttcttccaggaggataaagttgttcatggtggc | |
| | | gaccggtggatccc (SEQ ID NO: 47) | |
| | | 2a) | |
| | | cgggcccgggatccaccggtcgccaccatgaacaactttatcct | |
| | | cctggaagaacagctc (SEQ ID NO: 48) | |
| | | 2b) | |
| | | GATGGTGTCCCCCGCCACCTCCGCCACC | |
| | | TCCaagtcctgattctgcaatttcagccagtt (SEQ ID | |
| | | NO: 62) | |
| EG19 | FIG. 2R | 1a) cataatcagccataccacatttgtagaggttttacttgc | 5 |
| | | (SEQ ID NO: 73) | |
| | | 1b) | |
| | | taCTTGTACAGCTCGTCCATGCCGAGAG | |
| | | (SEQ ID NO: 74) | |
| | | 2a) | |
| | | CTCTCGGCATGGACGAGCTGTACAAGta | |
| | | (SEQ ID NO: 75) | |
| | | 2b) gcaagtaaaacctctacaaatgtggtatggctgattatg | |
| | | (SEQ ID NO: 76) | |
| EG20 | FIG. 2S | 1a) | 5 |
| | | tcctctctgcttctagaataaatcataatcagccataccacatttgta | |
| | | gaggttttacttgct (SEQ ID NO: 77) | |
| | | 1b) | |
| | | tgtcatgaatcagtaggtccgcaaagtaaccagcgtagtgCTT | |
| | | GTACAGCTCGTCCATGCCGAGAG (SEQ | |
| | | ID NO: 78) | |
| | | 2a) | |
| | | actttgcggacctactgattcatgacattgagacaaatccaggga | |
| | | tgaactttctacgtaagatagtgaaaaatt (SEQ ID NO: | |
| | | 79) | |
| | | 2b) | |
| | | acctctacaaatgtggtatggctgattatgatttattctagaagcag | |
| | | agaggaatctttg (SEQ ID NO: 80) | |
| EG21 | FIG. 2T | 1a) | 5 |
| | | gctggttactttgcggacctactgattcatgacattgagacaaatc | |
| | | cagggggattcggacaccaaaacaaagcggtgtacactg | |
| | | (SEQ ID NO: 81) | |
| | | 1b) | |
| | | aaacctctacaaatgtggtatggctgattatgatttgttccatggctt | |
| | | cttcttcgtaggcatacaagtc (SEQ ID NO: 82) | |
| | | 2a) | |
| | | tgtctcaatgtcatgaatcagtaggtccgcaaagtaaccagcgta | |
| | | gtgCTTGTACAGCTCGTCCATGCCGAGAG | |
| | | TGATCCC (SEQ ID NO: 83) | |
| | | 2b) | |
| | | gagacttgtatgcctacgaagaagaagccatggaacaaatcata | |
| | | atcagccataccacatttgtagaggttttacttgct (SEQ ID | |
| | | NO: 84) | |
| EG22 | FIG. 2U | 1a) | 4 |
| | | catggcagaggtgttcagagtcctcatggtggcgaccggtggat | |
| | | tcacgacacctgaaatggaagaaaaaaac (SEQ ID NO: | |
| | | 85) | |
| | | 1b) | |
| | | attaccgccatgcattagttattaggctccggtgcccgtcagtgg | |
| | | gcagagcg (SEQ ID NO: 86) | |
| | | 2a) | |
| | | agtttttttcttccatttcaggtgtcgtgaatccaccggtcgccacc | |
| | | atgaggactctgaacacctc (SEQ ID NO: 87) | |
| | | 2b) | |
| | | gtgcgctctgcccactgacgggcaccggagcctaataactaatg | |
| | | catggcggtaat (SEQ ID NO: 88) | |
| EG23 | FIG. 2V | 1a) | 4 |
| | | gaggccgaggccgcctcggcctctgagctaatccaccggtcgc | |
| | | caccatgaggactctgaacacctc (SEQ ID NO: 89) | |
| | | 1b) | |
| | | ataaccgtattaccgccatgcattagttattaggtgtggaaagtcc | |
| | | ccaggctccccagcaggcaga (SEQ ID NO: 90) | |
| | | 2a) | |
| | | ttcagagtcctcatggtggcgaccggtggattagctcagaggcc | |
| | | gaggcggcctcggcctct (SEQ ID NO: 91) | |
| | | 2b) | |
| | | tctgcctgctggggagcctggggactttccacacctaataactaa | |
| | | tgcatggcggtaatacggtta (SEQ ID NO: 92) | |
| EG24 | FIG. 2W | 1a) | 6 |
| | | GGAGGTGGCGGAGGTGGCGGGGGACA | |
| | | CCATCACCATCA (SEQ ID NO: 93) | |
| | | 1b) | |
| | | AGACAACGCTGGCCTTTTCCAGAGGCG | |
| | | ACCTCTGCATggtggcgaccggtggatcccgggcccg | |
| | | (SEQ ID NO: 94) | |
| | | 2a) | |
| | | cgggcccgggatccaccggtcgccaccATGCAGAGG | |
| | | TCGCCTCTGGAAAAGGCCAGCGTTGTC | |
| | | TC (SEQ ID NO: 95) | |
| | | 2b) | |
| | | CCCCGCCACCTCCGCCACCTCCAAGCCT | |
| | | TGTATCTTGCACCTCTTCTTCTGTCTCC | |
| | | (SEQ ID NO: 96) | |
| EG25 | FIG. 2X | 1a) | 6 |
| | | GGAGGTGGCGGAGGTGGCGGGGGACA | |
| | | CCATCACCATCA (SEQ ID NO: 93) | |
| | | 1b) | |
| | | AGACAACGCTGGCCTTTTCCAGAGGCG | |
| | | ACCTCTGCATggtggcgaccggtggatcccgggcccg | |
| | | (SEQ ID NO: 94) | |
| | | 2a) | |
| | | cgggcccgggatccaccggtcgccaccATGCAGAGG | |
| | | TCGCCTCTGGAAAAGGCCAGCGTTGTC | |
| | | TC (SEQ ID NO: 95) | |
| | | 2b) | |
| | | CCCCGCCACCTCCGCCACCTCCAAGCCT | |
| | | TGTATCTTGCACCTCTTCTTCTGTCTCC | |
| | | (SEQ ID NO: 96) | |

TABLE 3
Illustrative amino acid sequences comprised by some constructs

| | Illustrative | |
|---|---|---|
| Description | constructs | Amino acid sequence |
| DRD1-GFP | EG7, EG8, | MRTLNTSAMDGTGLVVERDFSVRILTACFLSLLILSTLLGN |
| | EG10, EG12, | TLVCAAVIRFRHLRSKVTNFFVISLAVSDLLVAVLVMPWK |
| | EG10, EG19, | AVAEIAGFWPFGSFCNIWVAFDIMCSTASILNLCVISVDRY |
| | EG20, EG21, | WATSSPFRYERKMTPKAAFILISVAWTLSVLISFIPVQLSWH |
| | EG22, EG23 | KAKPTSPSDGNATSLAETIDNCDSSLSRTYAISSSVISFYIPV |
| | | AIMIVTYTRIYRIAQKQIRRIAALERAAVHAKNCQTTTGNG |
| | | KPVECSQPESSFKMSFKRETKVLKTLSVIMGVFVCCWLPF |
| | | FILNCILPFCGSGETQPFCIDSNTFDVFVWFGWANSSLNPII |
| | | YAFNADFRKAFSTLLGCYRLCPATNNAIETVSINNNGAAM |
| | | FSSHHEPRGSISKECNLVYLIPHAVGSSEDLKKEEAAGIAR |
| | | PLEKLSPALSVILDYDTDVSLEKIQPITQNGQHPTGGGGSG |
| | | GGGSGGGGSMVSKGEELFTGVVPILVELDGDVNGHKFSV |
| | | SGEGEGDATYGKLTLKFICTTGKLPVPWPTLVTTLTYGVQ |
| | | CFARYPDHMKQHDFFKSAMPEGYVQERTIFFKDDGNYKT |
| | | RAEVKFEGDTLVNRIELKGIDFKEDGNILGHKLEYNYNSH |
| | | KVYITADKQKNGIKVNFKTRHNIEDGSVQLADHYQQNTPI |
| | | GDGPVLLPDNHYLSTQSKLSKDPNEKRDHMVLLEFVTAA |
| | | GITLGMDELYK |
| | | (SEQ ID NO: 12) |
| GFP | EG1, EG2, | MVSKGEELFTGVVPILVELDGDVNGHKFSVSGEGEGDAT |
| | EG3, EG7, | YGKLTLKFICTTGKLPVPWPTLVTTLTYGVQCFARYPDH |
| | EG8, EG10, | MKQHDFFKSAMPEGYVQERTIFFKDDGNYKTRAEVKFEG |
| | EG12, EG10, | DTLVNRIELKGIDFKEDGNILGHKLEYNYNSHKVYITADK |
| | EG19, EG20, | QKNGIKVNFKTRHNIEDGSVQLADHYQQNTPIGDGPVLLP |
| | EG21, EG22, | DNHYLSTQSKLSKDPNEKRDHMVLLEFVTAAGITLGMDE |
| | EG23 | LYK |
| | | (SEQ ID NO: 13) |
| DRD1-Strep | EG5, EG6 | MRTLNTSAMDGTGLVVERDFSVRILTACFLSLLILSTLLGN |
| | | TLVCAAVIRFRHLRSKVTNFFVISLAVSDLLVAVLVMPWK |
| | | AVAEIAGFWPFGSFCNIWVAFDIMCSTASILNLCVISVDRY |
| | | WAISSPFRYERKMTPKAAFILISVAWTLSVLISFIPVQLSWH |
| | | KAKPTSPSDGNATSLAETIDNCDSSLSRTYAISSSVISFYIPV |
| | | AIMIVTYTRIYRIAQKQIRRIAALERAAVHAKNCQTTTGNG |
| | | KPVECSQPESSFKMSFKRETKVLKTLSVIMGVFVCCWLPF |
| | | FILNCILPFCGSGETQPFCIDSNTFDVFVWFGWANSSLNPII |
| | | YAFNADFRKAFSTLLGCYRLCPATNNAIETVSINNNGAAM |
| | | FSSHHEPRGSISKECNLVYLIPHAVGSSEDLKKEEAAGIAR |
| | | PLEKLSPALSVILDYDTDVSLEKIQPITQNGQHPTTGTRPL |
| | | WSHPQFEK |
| | | (SEQ ID NO: 14) |
| ITK | EG9, EG17, | MNNFILLEEQLIKKSQQKRRTSPSNFKVRFFVLTKASLAYF |
| | EG18 | EDRHGKKRTLKGSIELSRIKCVEIVKSDISIPCHYKYPFQVV |
| | | HDNYLLYVFAPDRESRQRWVLALKEETRNNNSLVPKYHP |
| | | NFWMDGKWRCCSQLEKLATGCAQYDPTKNASKKPLPPT |
| | | PEDNRRPLWEPEETVVIALYDYQTNDPQELALRRNEEYCL |
| | | LDSSEIHWWRVQDRNGHEGYVPSSYLVEKSPNNLETYEW |
| | | YNKSISRDKAEKLLLDTGKEGAFMVRDSRTAGTYTVSVF |
| | | TKAVVSENNPCIKHYHIKETNDNPKRYYVAEKYVFDSIPL |
| | | LINYHQHNGGGLVTRLRYPVCFGRQKAPVTAGLRYGKW |
| | | VIDPSELTFVQEIGSGQFGLVHLGYWLNKDKVAIKTIREG |
| | | AMSEEDFIEEAEVMMKLSHPKLVQLYGVCLEQAPICLVFE |
| | | FMEHGCLSDYLRTQRGLFAAETLLGMCLDVCEGMAYLEE |
| | | ACVIHRDLAARNCLVGENQVIKVSDFGMTRFVLDDQYTS |
| | | STGTKFPVKWASPEVFSFSRYSSKSDVWSFGVLMWEVFSE |
| | | GKIPYENRSNSEVVEDISTGFRLYKPRLASTHVYQIMNHC |
| | | WKERPEDRPAFSRLLRQLAEIAESGLGGGGGGGGHHHHH |
| | | HV |
| | | (SEQ ID NO: 15) |
| C1 Inhibitor | EG15, EG16 | MASRLTLLTLLLLLLAGDRASSNPNATSSSSQDPESLQDR |
| | | GEGKVATTVISKMLFVEPILEVSSLPTTNSTTNSATKITANT |
| | | TDEPTTQPTTEPTTQPTIQPTQPTTQLPTDSPTQPTTGSFCP |
| | | GPVTLCSDLESHSTEAVLGDALVDFSLKLYHAFSAMKKV |
| | | ETNMAFSPFSIASLLTQVLLGAGENTKTNLESILSYPKDFT |
| | | CVHQALKGFTTKGVTSVSQIFHSPDLAIRDTFVNASRTLYS |
| | | SSPRVLSNNSDANLELINTWVAKNTNNKISRLLDSLPSDTR |
| | | LVLLNAIYLSAKWKTTFDPKKTRMEPFHFKNSVIKVPMM |
| | | NSKKYPVAHFIDQTLKAKVGQLQLSHNLSLVILVPQNLKH |
| | | RLEDMEQALSPSVFKAIMEKLEMSKFQPTLLTLPRIKVTTS |
| | | QDMLSIMEKLEFFDFSYDLNLCGLTEDPDLQVSAMQHQT |
| | | VLELTETGVEAAAASAISVARTLLVFEVQQPFLFVLWDQQ |
| | | HKFPVFMGRVYDPRA |
| | | (SEQ ID NO: 16) |
| T7 RNA | EG4 | MNTINIAKNDFSDIELAAIPFNTLADHYGERLAREQLALEH |
| polymerase | | ESYEMGEARFRKMFERQLKAGEVADNAAAKPLITTLLPK |
| | | MIARINDWFEEVKAKRGKRPTAFQFLQEIKPEAVAYITIKT |
| | | TLACLTSADNTTVQAVASAIGRAIEDEARFGRIRDLEAKH |
| | | FKKNVEEQLNKRVGHVYKKAFMQVVEADMLSKGLLGGE |
| | | AWSSWHKEDSIHVGVRCIEMLIESTGMVSLHRQNAGVVG |
| | | QDSETIELAPEYAEAIATRAGALAGISPMFQPCVVPPKPWT |
| | | GITGGGYWANGRRPLALVRTHSKKALMRYEDVYMPEVY |
| | | KAINIAQNTAWKINKKVLAVANVITKWKHCPVEDIPAIER |
| | | EELPMKPEDIDMNPEALTAWKRAAAAVYRKDKARKSRRI |
| | | SLEFMLEQANKFANHKAIWFPYNMDWRGRVYAVSMFNP |
| | | QGNDMTKGLLTLAKGKPIGKEGYYWLKIHGANCAGVDK |
| | | VPFPERIKFIEENHENIIVIACAKSPLENTWWAEQDSPFCFLA |
| | | FCFEYAGVQHHGLSYNCSLPLAFDGSCSGIQHFSAMLRDE |
| | | VGGRAVNLLPSETVQDIYGIVAKKVNEILQADAINGTDNE |
| | | VVTVTDENTGEISEKVKLGTKALAGQWLAYGVTRSVTKR |
| | | SVMTLAYGSKEFGFRQQVLEDTIQPAIDSGKGLMFTQPNQ |
| | | AAGYMAKLIWESVSVTVVAAVEAMNWLKSAAKLLAAE |
| | | VKDKKTGEILRKRCAVHWVTPDGFPVWQEYKKPIQTRLN |
| | | LMFLGQFRLQPTINTNKDSEIDAHKQESGIAPNFVHSQDGS |
| | | HLRKTVVWAHEKYGIESFALIHDSFGTIPADAANLFKAVR |
| | | ETMVDTYESCDVLADFYDQFADQLHESQLDKMPALPAK |
| | | GNLNLRDILESDFAFA |
| | | (SEQ ID NO: 17) |
| CF TR | EG24, EG25 | MQRSPLEKASVVSKLFFSWTRPILRKGYRQRLELSDIYQIP |
| | | SVDSADNLSEKLEREWDRELASKKNPKLINALRRCFFWRF |
| | | MFYGIFLYLGEVTKAVQPLLLGRIIASYDPDNKEERSIAIYL |
| | | GIGLCLLFIVRTLLLHPAIFGLHHIGMQMRIAMFSLIYKKTL |
| | | KLSSRVLDKISIGQLVSLLSNNLNKFDEGLALAHFVWIAPL |
| | | QVALLMGLIWELLQASAFCGLGFLIVLALFQAGLGRMMM |
| | | KYRDQRAGKISERLVITSEMIENIQSVKAYCWEEAMEKMI |
| | | ENLRQTELKLTRKAAYVRYFNSSAFFFSGFFVVFLSVLPY |
| | | ALIKGIILRKIFTTISFCIVLRMAVTRQFPWAVQTWYDSLG |
| | | AINKIQDFLQKQEYKTLEYNLTTTEVVMENVTAFWEEGF |
| | | GELFEKAKQNNNNRKTSNGDDSLFFSNFSLLGTPVLKDIN |
| | | FKIERGQLLAVAGSTGAGKTSLLMMIMGELEPSEGKIKHS |
| | | GRISFCSQFSWIMPGTIKENIIFGVSYDEYRYRSVIKACQLE |
| | | EDISKFAEKDNIVLGEGGITLSGGQRARISLARAVYKDADL |
| | | YLLDSPFGYLDVLTEKEIFESCVCKLMANKTRILVTSKME |
| | | HLKKADKILILHEGSSYFYGTFSELQNLQPDFSSKLMGCDS |
| | | FDQFSAERRNSILTETLHRFSLEGDAPVSWTETKKQSFKQT |
| | | GEFGEKRKNSILNPINSIRKFSIVQKTPLQMNGIEEDSDEPL |
| | | ERRLSLVPDSEQGEAILPRISVISTGPTLQARRRQSVLNLMT |
| | | HSVNQGQNIHRKTTASTRKVSLAPQANLTELDIYSRRLSQ |
| | | ETGLEISEEINEEDLKECLFDDMESIPAVTTWNTYLRYITV |
| | | HKSLIFVLIWCLVIFLAEVAASLVVLWLLGNTPLQDKGNS |
| | | THSRNNSYAVIITSTSSYYVFYIYVGVADTLLAMGFFRGLP |
| | | LVHTLITVSKILHHKMLHSVLQAPMSTLNTLKAGGILNRF |
| | | SKDIAILDDLLPLTIFDFIQLLLIVIGAIAVVAVLQPYIFVAT |
| | | VPVIVAFIMLRAYFLQTSQQLKQLESEGRSPIFTHLVTSLK |
| | | GLWTLRAFGRQPYFETLFHKALNLHTANWFLYLSTLRWF |
| | | QMRIEMIFVIFFIAVTFISILTTGEGEGRVGIILTLAMNIMST |
| | | LQWAVNSSIDVDSLMRSVSRVFKFIDMPTEGKPTKSTKPY |
| | | KNGQLSKVMIIENSHVKKDDIWPSGGQMTVKDLTAKYTE |
| | | GGNAILENISFSISPGQRVGLLGRTGSGKSTLLSAFLRLLNT |
| | | EGEIQIDGVSWDSITLQQWRKAFGVIPQKVFIFSGTFRKNL |
| | | DPYEQWSDQEIWKVADEVGLRSVIEQFPGKLDFVLVDGG |
| | | CVLSHGHKQLMCLARSVLSKAKILLLDEPSAHLDPVTYQII |
| | | RRTLKQAFADCTVILCEHRIEAMLECQQFLVIEENKVRQY |
| | | DSIQKLLNERSLFRQAISPSDRVKLFPHRNSSKCKSKPQIAA |
| | | LKEETEEEVQDTRL |
| | | (SEQ ID NO: 18) |
| DRD1 | | MRTLNTSAMDGTGLVVERDFSVRILTACFLSLLILSTLLGN |
| | | TLVCAAVIRFRHLRSKVTNFFVISLAVSDLLVAVLVMPWK |
| | | AVAEIAGFWPFGSFCNIWVAFDIMCSTASILNLCVISVDRY |
| | | WAISSPFRYERKMTPKAAFILISVAWTLSVLISFIPVQLSWH |
| | | KAKPTSPSDGNATSLAETIDNCDSSLSRTYAISSSVISFYIPV |
| | | AIMIVTYTRIYRIAQKQIRRIAALERAAVHAKNCQTTTGNG |
| | | KPVECSQPESSFKMSFKRETKVLKTLSVIMGVFVCCWLPF |
| | | FILNCILPFCGSGETQPFCIDSNTFDVFVWFGWANSSLNPII |
| | | YAFNADFRKAFSTLLGCYRLCPATNNAIETVSINNNGAAM |
| | | FSSHHEPRGSISKECNLVYLIPHAVGSSEDLKKEEAAGIAR |
| | | PLEKLSPALSVILDYDTDVSLEKIQPITQNGQHPT |
| | | (SEQ ID NO: 19) |
| NADase | | MTRPLLAVPGPDGGGGTGPWWAAGGRGPREVSPGAGTE |
| (SARM1) | | VQDALERALPELQQALSALKQAGGARAVGAGLAEVFQL |
| | | VEEAWLLPAVGREVAQGLCDAIRLDGGLDLLLRLLQAPE |
| | | LETRVQAARLLEQILVAENRDRVARIGLGVILNLAKEREP |
| | | VELARSVAGILEHMFKHSEETCQRLVAAGGLDAVLYWCR |
| | | RTDPALLRHCALALGNCALHGGQAVQRRMVEKRAAEWL |
| | | FPLAFSKEDELLRLHACLAVAVLATNKEVEREVERSGTLA |
| | | LVEPLVASLDPGRFARCLVDASDTSQGRGPDDLQRLVPLL |
| | | DSNRLEAQCIGAFYLCAEAAIKSLQGKTKVFSDIGAIQSLK |
| | | RLVSYSTNGTKSALAKRALRLLGEEVPRPILPSVPSWKEA |
| | | EVQTWLQQIGFSKYCESFREQQVDGDLLLRLTEEELQTDL |
| | | GMKSGITRKRFFRELTELKTFANYSTCDRSNLADWLGSLD |
| | | PRFRQYTYGLVSCGLDRSLLHRVSEQQLLEDCGIRLGVHR |
| | | ARILTAAREMLHSPLPCTGGKPSGDTPDVFISYRRNSGSQL |
| | | ASLLKVHLQLHGFSVFIDVEKLEAGKFEDKLIQSVMGARN |
| | | FVLVLSPGALDKCMQDHDCKDWVHKEIVTALSCGKNIVP |
| | | IIDGFEWPEPQVLPEDMQAVLTFNGIKWSHEYQEATIEKII |
| | | RFLQGRSSRDSSAGSDTSLEGAAPMGPT |
| | | (SEQ ID NO: 20) |

Cell Lines—Culturing and Transfection

HEK293 cells were used to illustrate the application of the present systems, methods, and compositions in human eukaryotic cells. HEK293 adherent cells (CLS) were cultured in Dulbecco's Modified Eagle Medium high glucose (Gibco) supplemented with 10% Fetal Bovine Serum (Gibco) and 50,000 U Pen Strep (Gibco). HEK293 cells were grown to 80% confluency at 37° C. and 5% CO₂ before transiently transfecting using 293 fectin (ThermoFisher) according to manufacturer's instruction. Protein-expressing cells were harvested after 48 h by detaching the cells using 0.5% trypsin solution for 5 min at 37° C. and scraping. Cells were pelleted (5,000×g, 15 min, 4° C.) and supernatant was discarded. Cell pellets were stored at −80° C. until further usage.

Suspension HEK293 cells were used to illustrate the application of the present systems, methods, and compositions in human eukaryotic cells. Suspension adapted HEK293 cells (CLS) were cultured in Expi293 Expression Medium (Gibco) supplemented. 1 day before transfection, cells were seeded at 1.75×10⁶ cells/ml and incubated at 37° C. and 5% CO₂ over night before transiently transfecting using Expi293 Expression System Kit (Gibco) according to manufacturer's instruction. Protein-expressing cells were harvested after 48 h-96 h by centrifugation (5,000×g, 15 min, 4° C.). In the case of soluble or membrane protein the supernatant was discarded, and cell pellets were stored at −80° C. until further usage. In the case of secreted proteins, the supernatant was immediately used for further purification.

CHO-K1 cells are used to illustrate the application of the present systems, methods, and compositions in eukaryotic animal cells. CHO-K1 adherent cells (CLS) were cultured in DMEM/F-12 GlutaMAX medium (Gibco) supplemented with 10% Fetal Bovine Serum (Gibco). CHO-K1 cells were grown to 80% confluency at 37° C. and 5% CO₂ before transiently transfecting using Lipofectamine LTX (ThermoFisher) according to manufacturer's instruction. Protein-expressing cells were harvested after 48 h by detaching the cells using 0.5% trypsin solution for 5 min at 37° C. and scraping. Cells were pelleted (5,000×g, 15 min, 4° C.) and supernatant was discarded. Cell pellets were stored at −80° C. until further usage.

SF9 cells were used to illustrate the application of the present systems, methods, and compositions in eukaryotic insect cells. SF9 suspension cells (CLS) were cultured in Sf9-900 III Medium(Gibco). SF9 cells were grown at 26° C. and 130 rpm before seeding into 6 well plates for transiently transfection using Cellfectin II (ThermoFisher) according to manufacturer's instruction. Protein expressing cells were harvested after 48 h by detaching and pelleting (5,000×g, 15 min, 4° C.) and supernatant was discarded. Cell pellets were stored at −80° C. until further usage.

### Example 1: GFP Expression in HEK293 Cells

CMV Promoter System

To demonstrate the influence of the introduction of the viral nuclear pore blocking proteins during an expression, HEK293 cells were transfected with either EG1, EG2 or co-transfected with EG3 and EG4 constructs (see Table 2 and FIG. 2 for construct details). The expression of the viral pore blocking proteins resulted in controlled regulation of protein expression. Consequently, the obtained GFP signal was decreased. The reason for the controlled regulation of the gene of interest that is in tandem with the pore blocking proteins is the mode of action of the viral protein. Without being bound by theory, a possible mechanism for protein regulation is that by expressing pore blocking proteins, nuclear export of mRNA may be inhibited and as a consequence the translation of the target protein will be downregulated. After stabilizing, the pore blocking proteins will be degraded and mRNA transport will resume. This again leads to the expression of both the target protein and the enhancer protein, e.g., a pore blocking protein. This tightly controlled feedback ensures stabilization and permanent expression of the target protein and prevents the usual regulation of eukaryotic cells that leads to a shut-down of protein expression.

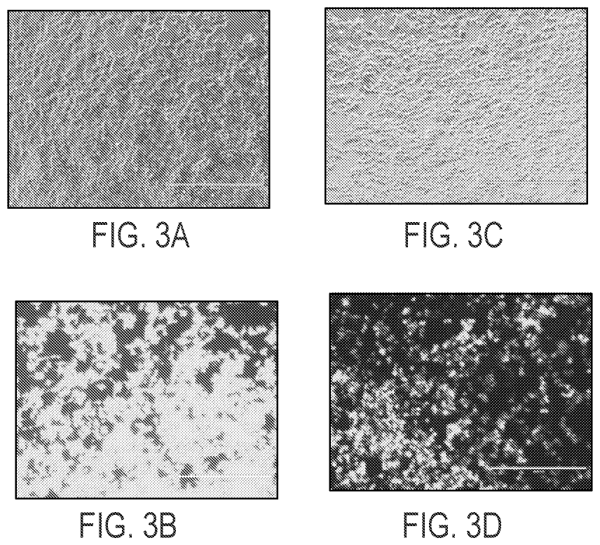

FIGS. 3A-3D show the effect on GFP expression in the absence and presence of the L-protein from ECMV as an illustrative enhancer protein according to the present disclosure. HEK293 cells were seeded at 0.05×10⁶ cells/well in a 24 well plate and incubated at 37° C. and 5% CO₂ overnight before transiently transfecting with either EG1 or EG2 as described above. GFP expression was monitored after 24 h and 48 h using fluorescence microscopy. Images were taken using a CCD Camera (Amscope) and analysed with ISCapture (Amscope). This example demonstrates the improved regulation of target protein expression in an illustrative system comprising a target protein polynucleotide and an enhancer protein polynucleotide according to the present disclosure.

T7 Polymerase System

While EG2 uses the natural polymerases of the eukaryotic host, other viral polymerases like T7 can be used to initiate transcription outside of the nucleus. The viral polymerase is under control of a standard eukaryotic promoter and the corresponding mRNA will depend on nuclear export. In the cytosol, the viral polymerase is translated and then initiates transcription of the target protein polynucleotide and the enhancer protein polynucleotide. In some embodiments, as a consequence of the expression of the enhancer proteins, the nuclear transport of the viral polymerase will decrease. The stabilization of the system will lead to degradation of the enhancer proteins and mRNA transport of the viral polymerase will resume. Without being bound by theory, this feedback may prevent the usual regulation of the cell while overexpressing a recombinant protein. In some circumstances, using viral polymerase gives the advantage of higher expression levels on a cell to cell basis compared to the system using eukaryotic polymerases.

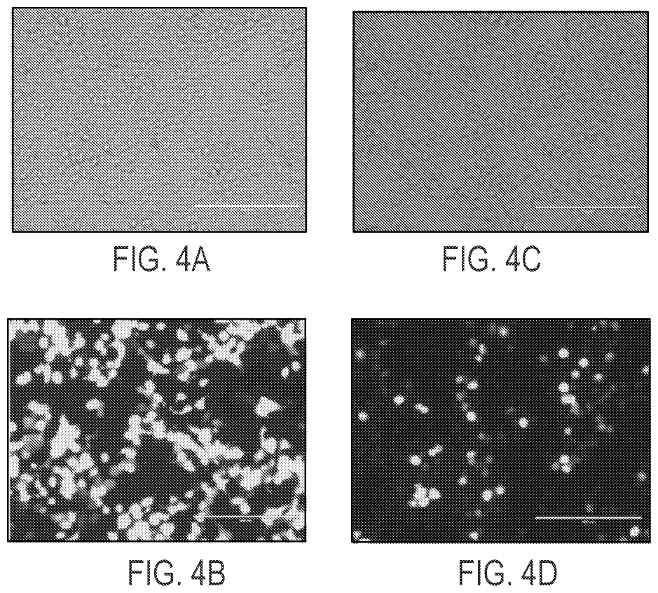

FIGS. 4A-4D show the successful expression of GFP in tandem with the L protein from ECMV from a T7 promoter when co-transfected with a T7 harboring vector. HEK293 cells were seeded at 0.05×10⁶ cells/well in a 24 well plate and incubated at 37° C. and 5% CO₂ overnight before transiently transfecting with either EG1 or EG3 and EG4 as described above. GFP expression was monitored after 24 h and 48 h using fluorescence microscopy. Images were taken using a CCD Camera (Amscope) and analyzed with ISCapture (Amscope). This example demonstrates the successful use of T7 as an illustrative viral polymerase in tandem with GFP as target protein and the L-protein of ECMV as enhancer protein. Similar to the example above, the introduction of the L-protein led to a tighter regulation of expression and therefore an overall reduction in over-expression.

### Example 2: Production of Dopamine Receptor 1 (DRD1)

DRD1 was used as to illustrate the application of the disclosed systems and methods to the co-expression of a membrane protein as target protein in combination with pore blocking proteins as enhancer proteins in order to yield a high density of active membrane receptors. DRD1 is a G-protein-coupled receptor and is known to be difficult to express using the academic standard. To visualize the correct translocation into the outer membrane of the cells, DRD1-GFP fusions (EG8) were used in the present system. To illustrate the problem with GPCRs in academic and industrial settings, the academic standard (EG10) was used as a control.

Improved Membrane Protein Expression and Membrane Localization

DRD1-GFP fusions were expressed in HEK293 cells. HEK293 cells were seeded at 0.05×10⁶ cells/well in a 24 well plate and incubated at 37° C. and 5% CO₂ overnight before transiently transfecting with either EG10 or EG8 as described above. DRD1-GFP expression was monitored after 24 h and 48 h using fluorescence microscopy. Images were taken using a CCD Camera (Amscope) and analyzed with ISCapture (Amscope).

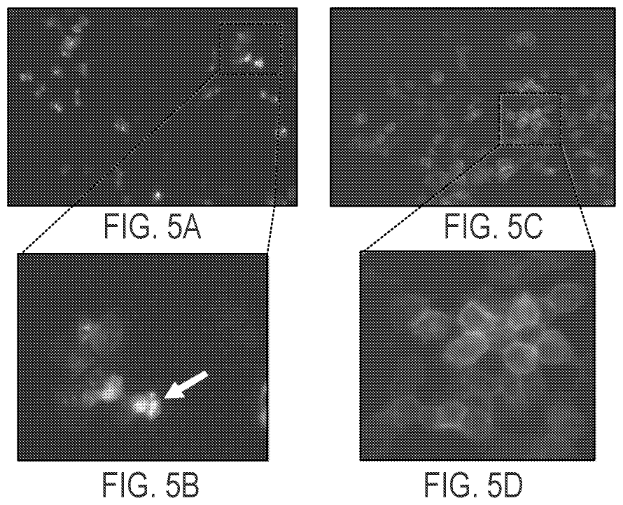

FIGS. 5A-5D demonstrate that EG10 fails to correctly translocate the expressed receptor. Without being bound by theory, it is believed that as a consequence of the overexpression of the human DRD1 receptor in human cells with the EG10 construct, the cells start to degrade or control the expressed target protein. This form of regulation results in the formation of denatured protein as inclusion bodies (FIG. 5B, red arrow). The control of expression of membrane proteins by the cells in this way may result in inactive and misfolded protein and consequently in unusable, poor quality expressed protein. In contrast, the co-expression of the target membrane protein with illustrative enhancer proteins resulted in correctly translocated DRD1-GFP, as can be seen by the correct insertion into the membrane and the absence of inclusion bodies (FIG. 5C-5D). This example demonstrates that the co-expression of an illustrative enhancer protein (the L-protein of ECMV) in conjunction with an illustrative target membrane protein (DRD1) resulted in improved expression and localization of the membrane protein. Without being bound by theory, it is believed that the present system produces tight regulation of target protein expression, thereby bypassing the normal regulation of the cell that would result in degradation of the expressed membrane protein. Thus, the present system is suitable for high yield expression and purification of GPCRs.

Expression of the Target Protein and the Enhancer Protein from Different Constructs

To illustrate that the enhancer protein can be encoded by a separate DNA molecule, DRD1-GFP (EG10) constructs were co-expressed with the L-protein from ECMV (EG11) under the control of a separate promoter on a separate vector. HEK293 cells were seeded at 0.05×10⁶ cells/well in a 24 well plate and incubated at 37° C. and 5% CO₂ overnight before transiently transfecting with EG10 and EG11 as described above. DRD1-GFP expression was monitored after 48 h using fluorescence microscopy. Images were taken and analyzed by an Echo Revolve microscopy system.

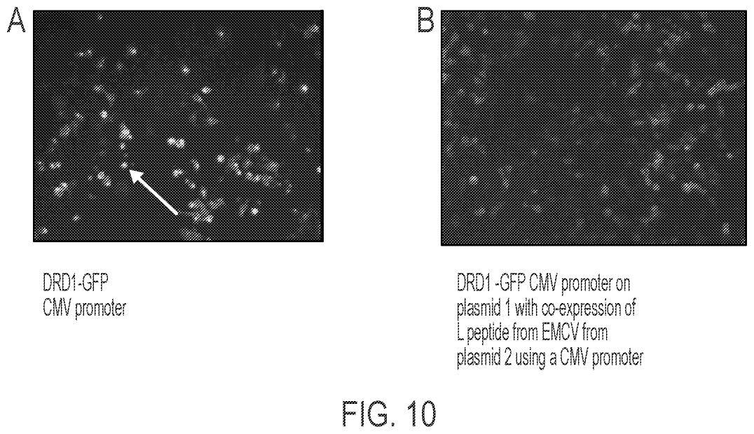

FIGS. 10A and B demonstrate that the co-expression of the L-protein with DRD1-GFP from two separate vectors ensures correct membrane association. While the expression of DRD1-GFP leads to the formation of inclusion bodies (FIG. 10A, red arrow), correct membrane association can be achieved by co-expression of the L-protein. FIG. 10B demonstrates that even when the L-protein is expressed from a separate vector and promoter, the regulatory effect of the L-protein is enough to restore the correct membrane association of DRD1.

These results demonstrate that the enhancer proteins disclosed herein and the target protein may be expressed from separate constructs to achieve the improvement in yield and/or functionality of the expressed target protein using the methods disclosed herein. Furthermore, these results suggest that the expression of any target protein from any construct or vector currently known or used in the art, in combination with the expression of one or more of the enhancer proteins disclosed herein, from the same construct or a different construct, can improve the yield and/or functionality of the expressed target protein. This dramatically enhances the versatility of the methods and compositions disclosed herein.

Functional Activity of the Membrane Protein

In addition to the illustration of a correctly translocated GPCR such as DRD1, activity tests were performed using a DRD1-Strep fusion. The smaller strep-tag ensures that the interaction with the cytosolic located G-protein is intact, and a functional assay can be performed. Upon binding of dopamine, DRD1 releases the heterotrimeric G-protein to its Gα subunit and its Gβγ complex. In the resting state, Gα binds GDP but upon activation exchanges GTP for GDP. The Gα-GTP complex interacts with adenylate cyclase (AC), resulting in activation of AC activity and consequently, increasing cAMP levels. Changes in intracellular cAMP levels can be measured by standard cAMP assays. The academic and industry standard (EG5) was compared to the same target protein in co-expression with the L-protein of ECMV.

DRD1-Strep fusions were expressed in HEK293 cells. HEK293 cells were seeded at 5,000 cells/well in a 96 well white clear bottom plates and incubated at 37° C. and 5% CO₂ overnight before transiently transfected with either EG5 or EG6 as described above. Protein was expressed for 48 h and DRD1 activity was analyzed using the cAMP-Glo™ assay (Promega) according to manufacturer's instructions. After 48 h, cells were washed with sterile PBS pH 7.2 and cells were incubated for 2 h with 20 μl of a 1 mM dopamine substrate solution (+dopamine; ON) or PBS pH 7.2 (−dopamine; OFF) at 37° C. After incubation, cells were washed with PBS pH 7.2 followed by addition of 20 μl lysis buffer. Lysis was performed for 15 min at room temperature (RT) with shaking. Subsequently, 40 μl detection solution was added and cells were incubated for 20 min at RT with shaking. Reactions were stopped using 80 μl Kinase-Glo® Reagent incubated for 15 min at RT before analyses. Luminescence was measured using a plate reader (BioTek Synergy™ LX) and data were analyzed using standard analysis programs.

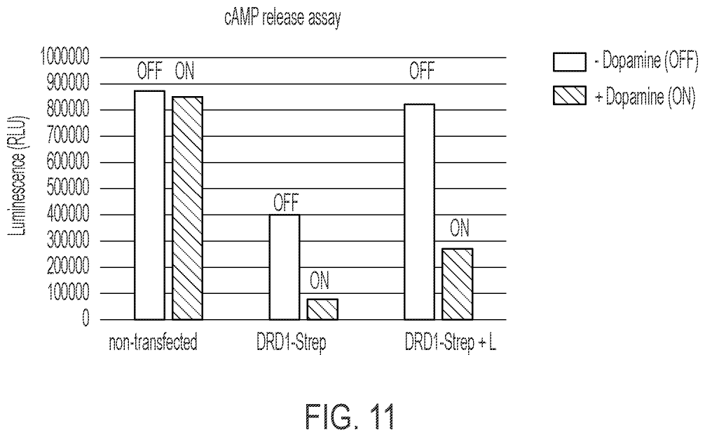

FIG. 11 demonstrates the advantage of expressing DRD1-Strep in tandem with the L protein from EMCV. When dopamine is added to cells expressing DRD1, the corresponding luminescence signal drops as result of internal cAMP release. FIG. 11 shows that by co-expressing DRD1 with the L protein from EMCV, there is a strong activating signal, as indicated by the difference between the OFF state, in the absence of dopamine, and the ON state, in the presence of dopamine. An important aspect of the assay is to exclude false activation of DRD1 or cAMP release in absence of the activator, dopamine. If the assay produces “leaky” signals, the usability of it for drug discovery screening is low. FIG. 11 shows that that by co-expressing DRD1 with the L protein from EMCV, “leaky” activation and therefore false negative readouts are greatly reduced when comparing just the OFF signals to non-transfected cells. Accordingly, the co-expression of the enhancer protein using the methods disclosed herein results in a tighter regulation of the activation of the target DRD1 protein. Therefore, the methods disclosed herein have applicability in drug discovery screening.

### Example 3: Expression of DRD1-GFP Using a Viral Promoter in Combination with a Viral Polymerase

For this example, DRD1-GFP, as an illustrative difficult-to-express target membrane protein was expressed using a T7 promoter to demonstrate that viral polymerases like T7 can be used to initiate transcription outside of the nucleus. As in Example 1, the viral polymerase was under control of a standard eukaryotic promoter and the corresponding mRNA relied on nuclear export.

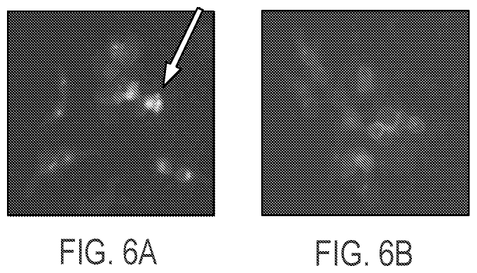

FIGS. 6A-6B demonstrates the successful expression of DRD1-GFP in tandem with the L protein from ECMV from a T7 promoter when co-transfected with a T7 harboring vector. HEK293 cells were seeded at 0.05×10⁶ cells/well in a 24 well plate and incubated at 37° C. and 5% CO₂ overnight before transiently transfecting with either EG10 or EG12 and EG4. DRD1-GFP expression was monitored after 24 h and 48 h using fluorescence microscopy. Images were taken using a CCD Camera (Amscope) and analyzed with ISCapture (Amscope). This example demonstrates the successful use of T7 as viral polymerase in tandem with DRD1-GFP as target protein and the L-protein of ECMV as enhancer protein.

### Example 4: Expression of DRD1-GFP Using Different Mammalian Promoters

Systems, methods, and compositions according to the present disclosure are compatible with a wide variety of mammalian promoters. To demonstrate the compatibility of the co-expression of the target protein and the enhancer protein from different promoters, DRD1-GFP was used as an illustrative target protein. As described in Example 2, the correct expression and translocation of DRD1-GFP can be easily detected by fluorescence microscopy. The constructs used in the experiment were engineered to express DRD1 from either CMV promoter (EG8), EF1-α promoter (EG22) or SV40 promoter (EG23), and to have the following elements—the nucleic acid sequence encoding DRD1-GFP, the nucleic acid sequence encoding IRES and the nucleic acid sequence encoding the L protein sequence. The academic standard systems (EG10) was used to illustrate the difference between correct and incorrect membrane association.

DRD1-GFP fusions under the control of different mammalian promoters were expressed in HEK293 cells. HEK293 cells were seeded at 0.05×10⁶ cells/well in a 24 well plate and incubated at 37° C. and 5% CO₂ overnight before transiently transfected with either EG8, EG10, EG22 or EG23 as described above. DRD1-GFP expression was monitored after 48 h using fluorescence microscopy. Images were taken and analyzed by an Echo Revolve microscopy system.

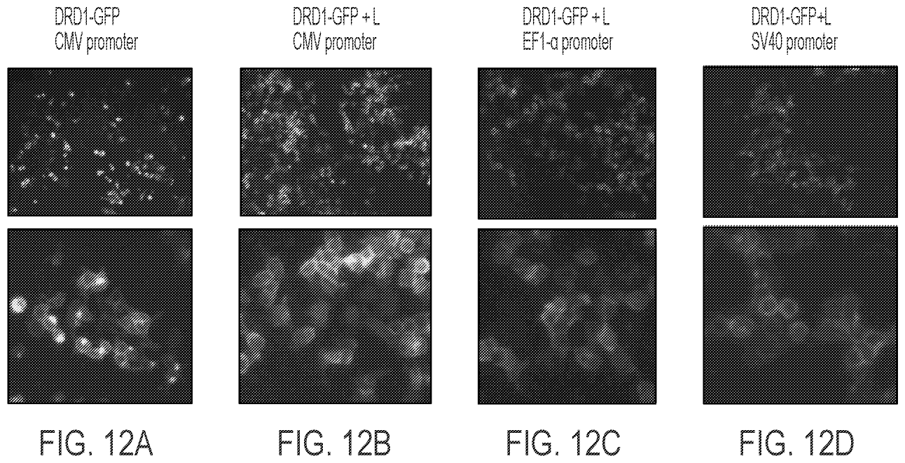

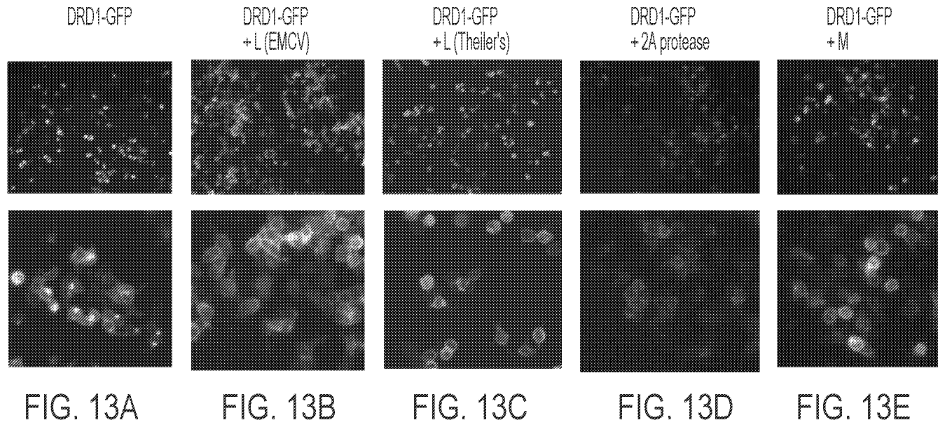

FIG. 12 demonstrates that different promoters may be used to drive target protein expression, in combination with the expression of the enhancer protein. While the expression of DRD1-GFP from the control construct shows that DRD1 fails to localize to the outer membrane of the cells, but rather localizes to inclusion bodies (bright green spots, FIG. 12A), DRD1-GFP that is expressed in combination with L-protein enhancer expressed from CMV, EF la and SV40 (FIGS. 12B-D) promoters are all correctly associated with the membrane judged by the absence of inclusion bodies. As expected, the different promoters result in different expression levels and therefore the amount of DRD1-GFP in the membrane (total amount of fluorescence) varies.

### Example 5: Expression of DRD1-GFP Using Different Viral Pore Blocking Proteins

DRD1-GFP, the illustrative target fusion protein was expressed in combination with different enhancer proteins in HEK293 cells. Constructs used in this experiment encoded DRD1-GFP and one of the enhancer proteins selected from the Leader protein of ECMV (EG8), the

Leader protein of Theiler's virus (EG19), the 2A protease of Polio virus (EG21) and the M protein of vesicular stomatitis virus (EG20). As described in Example 2, the correct expression and translocation of DRD1-GFP can be easily detected by fluorescence microscopy. The academic standard systems (EG10) was used to illustrate the difference between correct and incorrect membrane association. HEK293 cells were seeded at 0.05×10⁶ cells/well in a 24 well plate and incubated at 37° C. and 5% CO₂ overnight before being transiently transfected with either EG8, EG10, EG19, EG20 or EG21 as described above. DRD1-GFP expression was monitored after 48 h using fluorescence microscopy. Images were taken and analyzed by an Echo Revolve microscopy system.

FIG. 13 demonstrates that the Leader protein of ECMV (FIG. 13B), the Leader protein of Theiler's virus (FIG. 13C), the 2A protease of Polio virus (FIG. 13D) and the M protein of vesicular stomatitis virus (FIG. 13E) are all sufficient to ensure a correct membrane incorporation of DRD1-GFP in contrast to the DRD1-GFP without any of the enhancer proteins (FIG. 13A).

These results show that several different viral pore blocking proteins share the capability of improving the yield, localization, and/or functionality of the target protein, when expressed along with a target protein in a host cell. Without being bound to theory, it is thought that the blockage of the nuclear pore resulting from the expression from any one of these enhancer proteins might bypass the normal regulation of the cell that would have resulted in the degradation of the expressed target protein. Thus, this common mechanism by which a viral pore blocking protein enhances target protein expression, localization and activity allows the methods disclosed herein to be practiced with any pore blocking protein known in the art, discovered in the future, or disclosed herein.

### Example 6: Expression of DRD1-GFP in CHO Cells

The experiment of Example 2 was repeated using CHO-K1 (Chinese Hamster Ovary) cells instead of HEK293. DRD1-GFP was expressed from the EG19 construct, which also encodes an enhancer protein, or from the control EG10 construct.

DRD1-GFP fusions proteins were expressed in CHO-K1 cells. CHO-K1 cells were seeded at 0.05×10⁶ cells/well in a 24 well plate and incubated at 37° C. and 5% CO₂ overnight before transiently transfecting with either EG10 or EG19 using Lipofectamine 3000 (Thermofisher) according to manufactures instructions. DRD1-GFP expression was monitored after 48 h using fluorescence microscopy. Images were taken and analyzed by an Echo Revolve microscopy system.

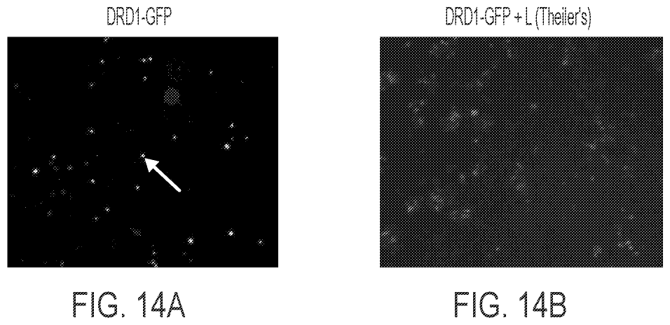

FIG. 14 demonstrates that EG10 fails to correctly translocate the expressed receptor. Interestingly, the consequence of the overexpression of the human DRD1 receptor in CHO cells seems to be more severe compared to HEK cells. With the EG10 construct, the cells start to degrade or control the expressed target protein resulting in the formation of denatured protein as inclusion bodies (FIG. 14A, red arrow). The control of expression of membrane proteins by the cells in this way may result in inactive and misfolded protein and consequently in unusable, poor quality expressed protein. In contrast, the co-expression of the target membrane protein with illustrative enhancer proteins resulted in correctly translocated DRD1-GFP, as can be seen by the correct insertion into the membrane and the absence of inclusion bodies (FIG. 14B). This example demonstrates that the co-expression of an illustrative enhancer protein (the L-protein of Theiler's virus) in conjunction with an illustrative target membrane protein (DRD1) results in improved expression and localization of the membrane protein. Additionally, this Example demonstrates that various eukaryotic cell types (for example, HEK293 or CHO cells) may be used in the practice of the disclosed methods.

### Example 7: Production of Expression of DRD1-GFP in Sf9 Cells

The experiment of Example 2 was repeated using Sf9 (Spodoptera frugiperda) cells instead of HEK293. DRD1-GFP was expressed from the EG8 construct or the industrial and academic standard construct, EG10.

DRD1-GFP fusions were expressed in Sf9 cells. Sf9 cells were seeded at 0.4×10⁶ cells/well in a 6 well plate and incubated for 15 min at RT before transiently transfecting with either EG10 or EG8 using Cellfectin Reagent II (Thermofisher) according to manufactures instruction. DRD1-GFP expression was monitored after 72 h using fluorescence microscopy. Images were taken and analyzed by an Echo Revolve microscopy system.

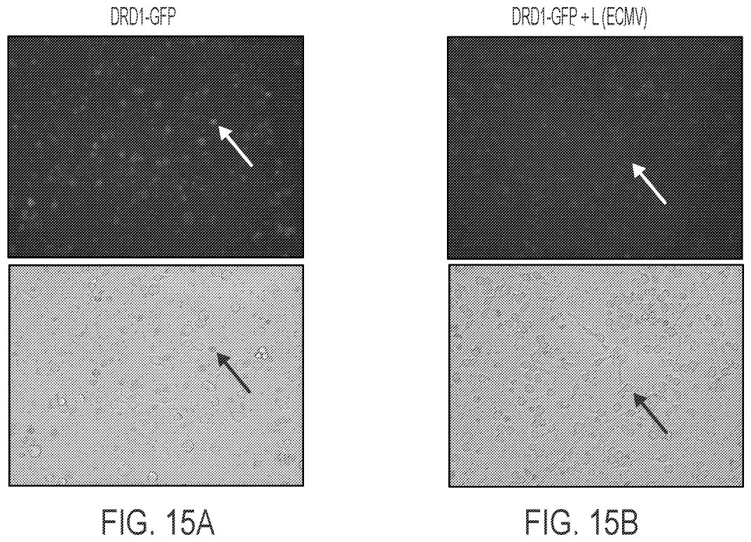

FIG. 15 demonstrates that EG10 not only fails to correctly translocate the expressed receptor but that the expressed receptors are highly toxic for the cells. The highest fluorescence signal was observed in cells that died as result of the toxicity of the expressed gene (FIG. 15A, red arrow). In contrast, the expression of DRD1-GFP using the disclosed methods prevents cell toxicity caused by the expression of DRD1-GFP and membrane-incorporated receptors are observed (FIG. 15B, red arrow). Interestingly, the consequence of the overexpression of the human DRD1 receptor in Sf9 cells seems to be more severe compared to HEK cells. Unregulated expression as in the standard system EG10 provokes a high cell death and as result unusable protein. The toxic effect is dramatically milder when expressing DRD1-GFP and L protein from EG8, as obvious by the overall cell health and the membrane bound receptors. This example demonstrates that the co-expression of an illustrative enhancer protein (the L-protein of EMCV) in conjunction with an illustrative target membrane protein (DRD1) resulted in improved expression and localization of the membrane protein with clearly improved control of toxic effect. Additionally, this example demonstrates that the disclosed methods are compatible with various eukaryotic cell types.

### Example 8: Production of IL2 Inducible T Cell Kinase (ITK)

ITK was used as an illustrative target protein to exemplify the application of the disclosed systems to express soluble proteins that are typically difficult to express. ITK is a member of the TEC family of kinases and is believed to play a role in T-cell proliferation and differentiation in T-cells. Also, ITK was used to demonstrate the consistency in enzyme activity between batches and the scalability of the methods disclosed herein. ITK was expressed in 3×10 ml, 100 ml, and 1000 ml growth medium. Additionally, an ITK-L-his protein fusion construct (EG9) was used to demonstrate that enhancer proteins can be fused to the recombinantly expressed target proteins without losing the ability to control the regulation. ITK-his fusions were expressed from the EG17, and from the academic and industrial standard (EG18) as comparison.

ITK-his and ITK-L-his fusions were expressed in HEK293 cells. HEK293 cells were seeded at 2×10⁶ cells/ml in 10 ml, 100 ml or 1000 ml Expi293 medium and incubated at 37° C., 120 rpm and 5% CO₂ overnight before transiently transfecting with either EG9, EG17 or EG18 as described above. Cells were harvested after 48 h (5,000×g, 15 min, 4 C) and cell pellets were stored at −80° C. until further usage.

To purify ITK, cells were resuspended in lysis buffer (40 mM Tris,7.5; 20 mM MgCl₂; 0.1 mg/ml BSA; 50 μM DTT; and 2 mM MnCl₂, protease inhibitor, DNAse), lysed by sonication (2 min, 10 s ON, 10 s OFF, 40% Amplitude) and crude cell extract was cleared (5,000×g, 20 min, 4° C.). A 5 ml His-resin column (GE Healthcare HisTrap) was equilibrated with wash buffer (40 mM Tris,7.5; 20 mM MgCl₂; 0.1 mg/ml BSA; 50 μM DTT; and 2 mM MnCl2) prior to loading to the cleared lysate using a peristaltic pump. After loading, the purification was performed on an ÄKTA™ system (Cytiva Life Sciences (former

GE Healthcare)). The column was washed with 5CV wash buffer before eluting with a continuous gradient 0-100% elution buffer (wash buffer+300 mM imidazole) over 25 CV. Protein containing fraction were analyzed by SDS-PAGE (6-12% BOLT, ThermoFisher) and protein containing fractions were pooled and concentrated.

Protein was further purified by size-exclusion chromatography (SEC) (Superdex 200, ThermoFisher) using SEC-Buffer (40 mM Tris,7.5; 20 mM MgCl₂, 150 mM NaCl) and fraction was analyzed by SDS-PAGE (6-12% BOLT, ThermoFisher). Protein containing fractions were pooled according to their appearance and analyzed for activity using the ITK Kinase Enzyme system in combination with ADP-Glo™ Assay (Promega) according to manufacturer's instructions. In short, full length ITK expressed from EG17 and EG18 were used in the assay with total enzyme concentrations of 200 ng, 100 ng, 50 ng and 0 ng. Substrate PolyE4Y1 was used in a concentration of 0.2 μg/μl and ATP was added to the reaction at 25 μM. In a 96 well plate, 5 μl Reaction buffer (as supplied with the kit) was combined with 10 μl of the Enzyme dilutions and 10 μl of the ATP/PolyE4Y1 mix. The plate was incubated for 60 min at RT. 25 μl ADP-Glo Reagent was added and the plate was again incubated for 40 min at RT. The reaction was stopped by adding 50 μl Kinase detection reagent and incubating for another 30 min at RT. The reaction was read by luminescence with a integration time of 1 s.

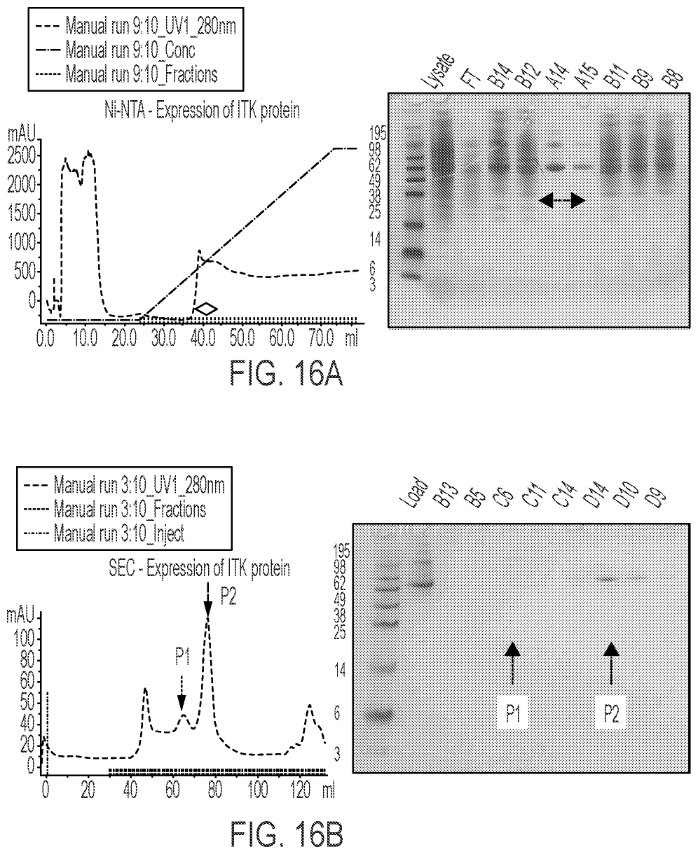

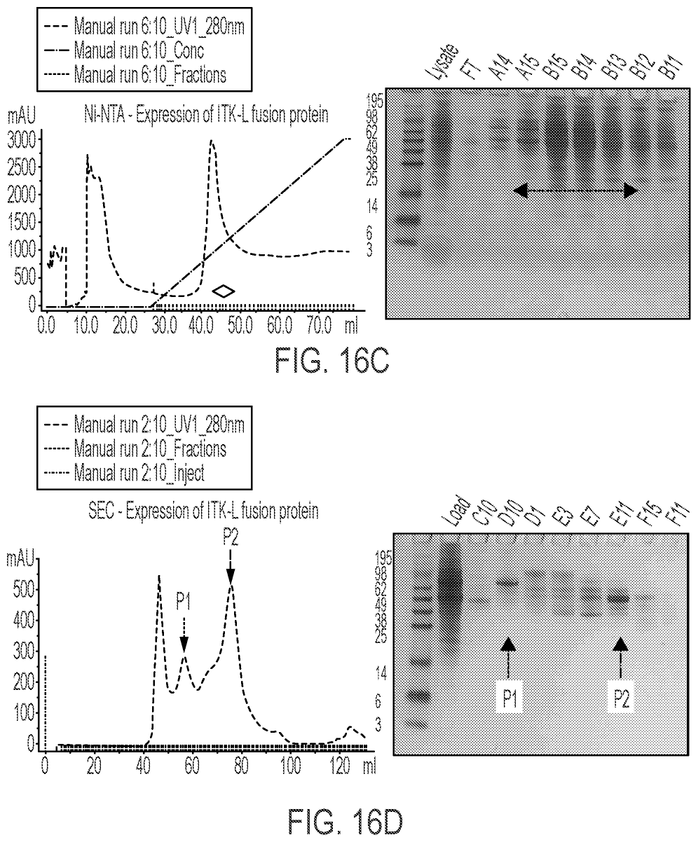

FIG. 16 shows the purification process for ITK protein, and for ITK protein fused with the enhancer protein L. During the purification using SEC two peaks (P1 and P2) could be identified as target protein that could be identified by western blot as monomeric (P2) and dimeric (P1) species (data not shown). Without being bound to theory, it is believed that ITK needs to form dimers to achieve an active form. ITK is a known kinase that is toxic to cells when over-expressed. Hence, the higher the activity of ITK, the more the expression will be down regulated by the host cell or rendered into a monomeric inactive form.

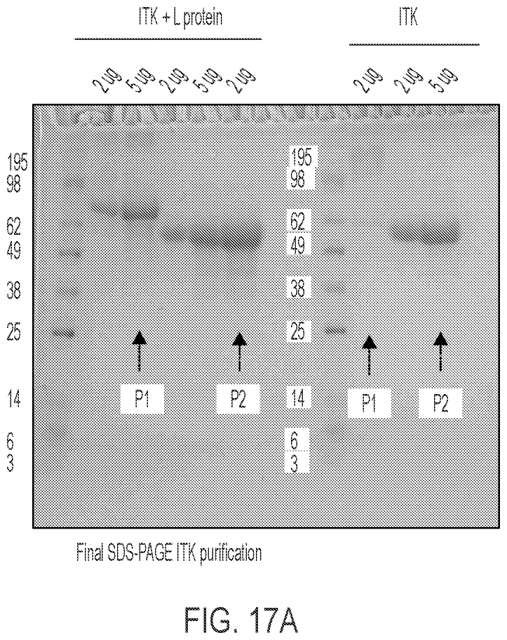

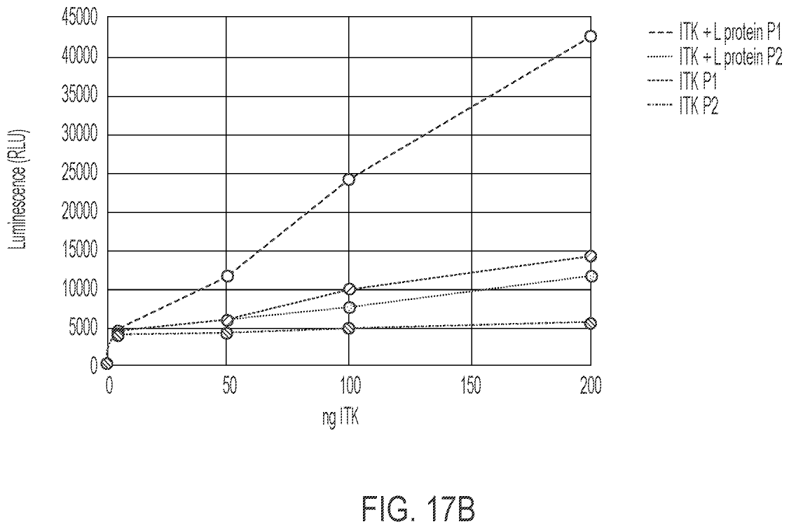

FIG. 17A shows the final SDS-PAGE of the purification of the identified species. Note that only P1 species is active and therefore the expression of an enhancer protein in combination with ITK leads to a huge increase of expression of the active ITK species. FIG. 17B demonstrates the difference in activity by using luminescence as the primary readout. Only P1 expressed from EG17 demonstrates a high activity and therefore is the only usable protein for drug screening against this kinase. Whereas both systems seem to express similar amount of the proteins of interest, ITK expressed using the methods disclosed herein shows more activity than the ITK protein expressed in the absence of an enhancer protein. This example demonstrates that the methods disclosed herein can be used to produce active protein that otherwise would be toxic or rendered inactive by the host cell. Furthermore, the disclosed methods can be used to not only produce active proteins that would be otherwise toxic but these proteins can then be used in drug screening such as small molecule screening to discover novel therapeutics.

### Example 9: Production of IL2 Inducible T Cell Kinase (ITK) in CHO-K1 Cells

The experiment of Example 8 was repeated using CHO cells instead of HEK293. ITK-his was expressed from EG17, or the control construct, EG18.

ITK-his fusions were expressed in CHO-K1 cells. In total 8 150 mm plates of each construct of CHO-K1 cells were seeded at 5×10⁶ cells/per dish and incubated at 37° C., and 5% CO₂ overnight before transiently transfecting with either EG17 or EG18 using Lipofectamine 3000 (Thermofisher) according to manufactures instruction. Cells were harvested after 48 h by scraping and spun down to remove the supernatant (5,000×g, 15 min, 4 C). Cell pellets were stored at −80° C. until further usage. To purify ITK, cells were resuspended in lysis buffer (40 mM Tris,7.5; 20 mM MgCl₂; 0.1 mg/ml BSA; 50 μM DTT; and 2 mM MnCl₂, protease inhibitor, DNAse), lysed by sonication (2 min, 10 s ON, 10 s OFF, 40% Amplitude) and crude cell extract was cleared (5,000×g, 20 min, 4° C.). A 5 ml His-resin column (GE Healthcare HisTrap) was equilibrated with wash buffer (40 mM Tris,7.5; 20 mM MgCl₂; 0.1 mg/ml BSA; 50 μM DTT; and 2 mM MnCl2) prior to loading to the cleared lysate using a peristaltic pump. After loading, the purification was performed on an AEKTA system. The column was washed with 5CV wash buffer before eluting with a continuous gradient 0-75% elution buffer (wash buffer+300 mM imidazole) over 20 CV. The elution was completed by 5 CV 100% elution buffer.

Protein containing fractions were analyzed by SDS-PAGE (6-12% SurePAGE, Bis-Tris, GenScript) and protein containing fractions were pooled and concentrated. Protein was further polished by size-exclusion chromatography (SEC) (Superdex 200, ThermoFisher) using SEC-Buffer (40 mM Tris,7.5; 20 mM MgCl₂, 150 mM NaCl) and fraction were analyzed by SDS-PAGE (6-12% SurePAGE, Bis-Tris, GenScript). Protein containing fractions were pooled according to their appearance and analyzed for activity using the ITK Kinase Enzyme system in combination with ADP-Glo Assay™ (Promega) according to manufacturer's instructions.

AITK expressed in Sf9 insect cells was used as standard. ΔITK as well as full length ITK expressed from EG17 and EG18 were used in the assay with total enzyme concentrations of 200 ng, 100 ng, 50 ng and 0 ng. Substrate PolyE4Y1 was used in a concentration of 0.2 μg/μl and ATP was added to the reaction at 25 μM. In a 96 well plate, 5 μl Reaction buffer (as supplied with the kit) was combined with 10 μl of the Enzyme dilutions and 10 μl of the ATP/PolyE4Y1 mix. The plate was incubated for 60 min at RT. 25 μl ADP-Glo Reagent was added and the plate was again incubated for 40 min at RT. The reaction was stopped by adding 50 μl Kinase detection reagent and incubating for another 30 min at RT. The reaction was read by luminescence with a integration time of 1 s.

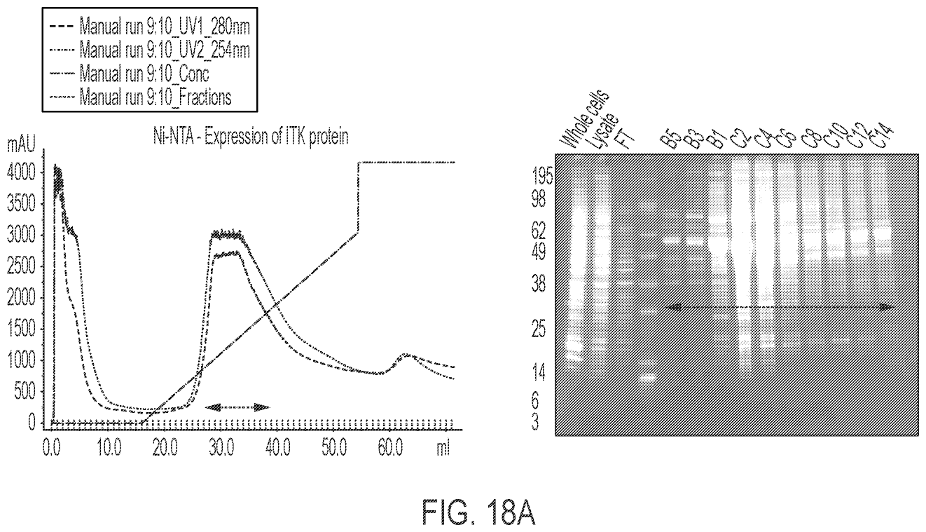

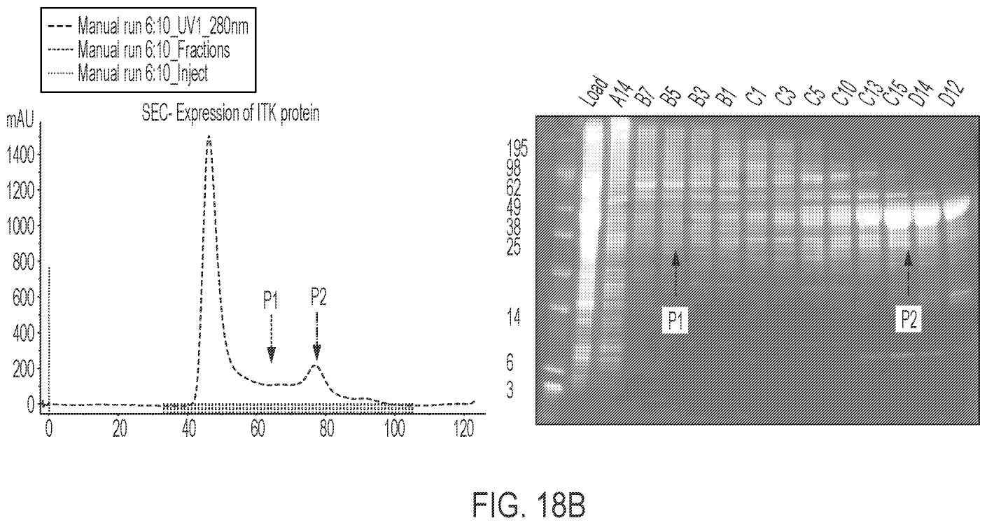

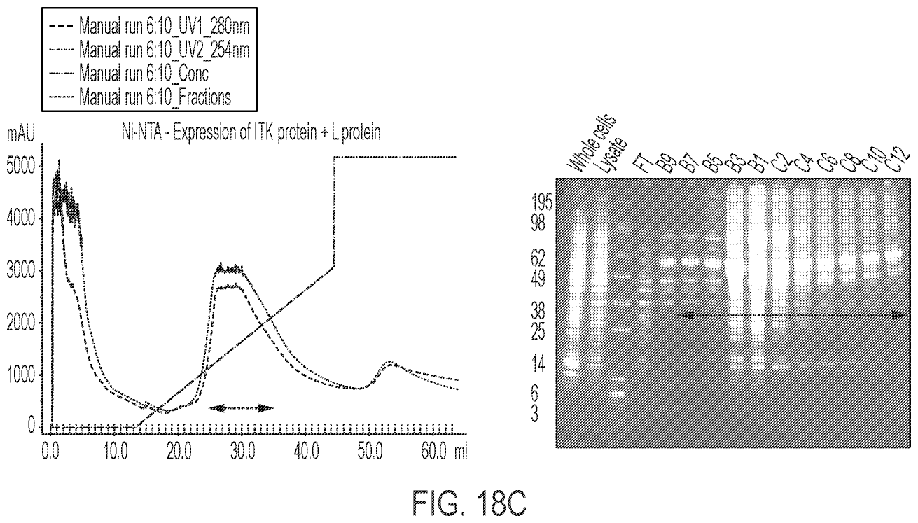

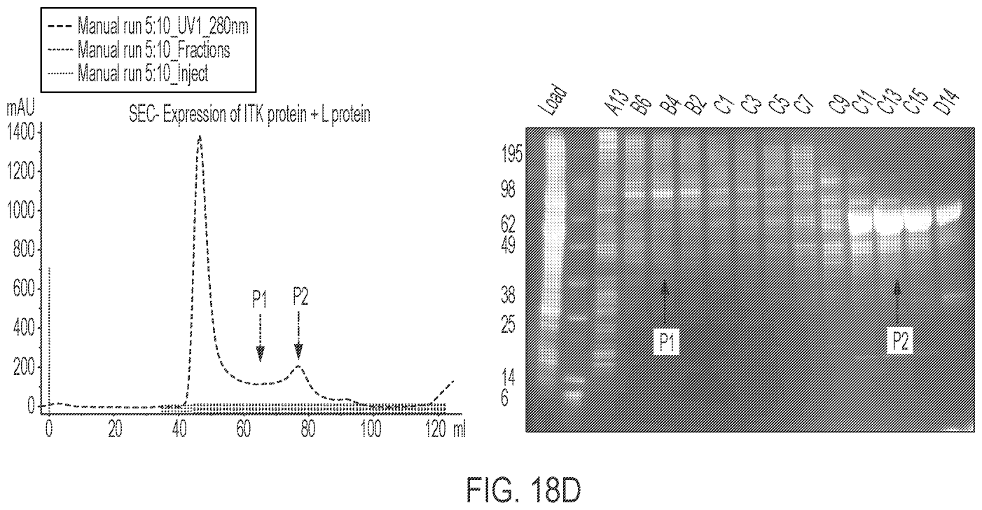

FIG. 18 shows the purification process of ITK expressed with and without the enhancer protein L. As mentioned above, during the purification using a SEC two peaks (P1 and P2) could be identified as target protein. Without being bound to theory, it is believed that ITK needs to form dimers to achieve an active form. ITK is a known kinase that is toxic to cells when over-expressed. Hence, the higher the activity of ITK the more the expression will be down regulated by the host cell or rendered into a monomeric inactive form.

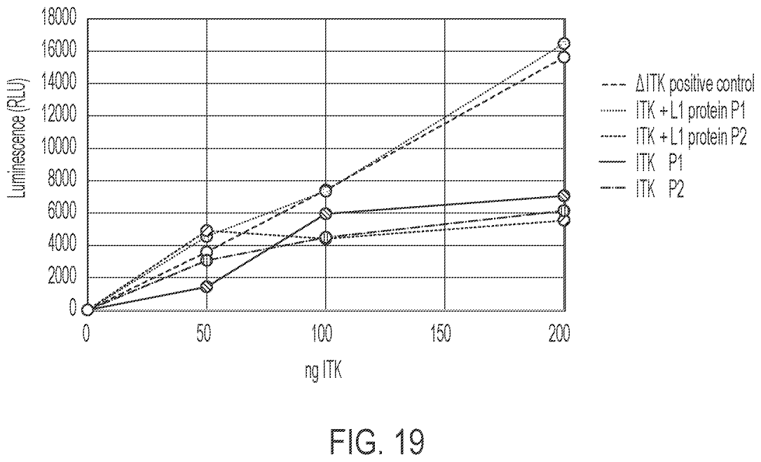

FIG. 19 demonstrates the difference in activity by using luminescence as the primary readout. Only P1 expressed from EG17 demonstrates a compatible activity to the provided AITK positive control. Whereas both systems seem to express similar amount of the proteins of interest, just the presented system achieves to produce active protein by controlling the regulation of the host cell. This example demonstrates that the methods disclosed herein can be used to produce active protein that otherwise would be toxic or rendered inactive by the host cell.

### Example 10: Production of IL2 Inducible T Cell Kinase (ITK) in Sf9 Cells

Example 8 is repeated using Sf9 cells instead of HEK293. ITK-his is expressed from the EG17 construct or from the industrial and academic standard EG18 construct. Expression in Sf9 cells is performed as described in Example 7, and protein purification of His-tagged ITK protein is done as described in Examples 8 and 9.

### Example 11: Expression of Cystic Fibrosis Transmembrane Conductance Regulator (CFTR)

CFTR was used as an additional example to demonstrate that the co-expression of a membrane protein as target protein in combination with pore blocking proteins as enhancer proteins yielded a high density of active ion-channel. CFTR is a transmembrane transporter of the ABC-transporter class that conducts chloride ions across epithelial cell membranes. CFTR is known to express in a heterogenous manner when using the academic standard (EG24). Heterogeneity increases the difficulty in purifying or analyzing the ABC transporter. To demonstrate the improvement of homogeneity, CFTR was either cloned into the backbone of an illustrative system (EG25) or was used as a PCR product. As comparison, the academic standard (EG24) was used alongside as a control.

CFTR constructs were expressed in HEK293 cells. HEK293 cells were seeded at 0.3×10⁶ cells/well in a 6 well plate and incubated at 37° C. and 5% CO₂ overnight before transiently transfecting with either EG25, the PCR-product of EG25 insert or EG24 as described above. CFTR expression was monitored after 24 h and 48 h using microscopy. Cells were harvested and lysed after 48 h using RIPA (Radio-Immunoprecipitation Assay) Buffer (CellGene). Lysate was cleared and analyzed by SDS-PAGE (6-12% BOLT, ThermoFisher) followed by Western blot (Nitrocellulose membrane, ThermoFisher) using anti-CFTR (Abcam, 2^{nd} antibody—anti-mouse-HRP).

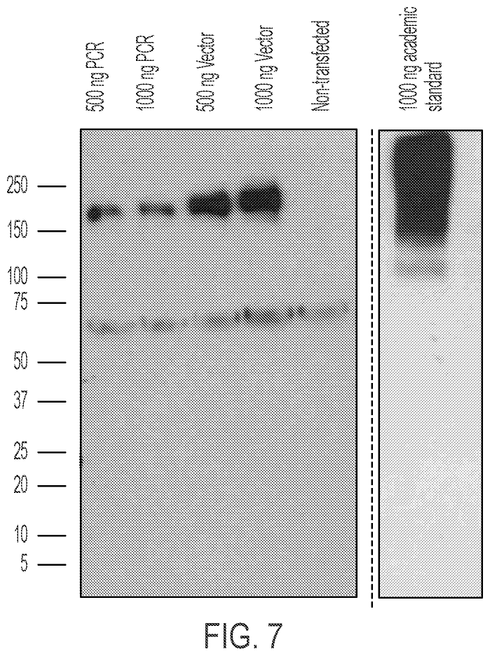

FIG. 7 demonstrates the impact of the co-expression of the L-protein with the CFTR. Whereas the academic standard produced a wide band on the Western blot, transcription and translation based on the EG25 construct resulted in defined bands demonstrating a highly homogenous expression of the ABC-transporter. Additionally, this example demonstrates that the expression system can be delivered into the cell as a vector or as a PCR product.

### Example 12: Expression of an NADase

An NADase was used as an illustrative target protein to exemplify the application of the disclosed systems for difficult-to-express, toxic soluble proteins. NADases are enzymatic proteins that catalyze the reaction from NAD+ to ADP-ribose and nicotinamide. Overexpression of an NADase normally leads to increased cell death due to the fact that the cell is stripped from its natural energy source NAD+. To demonstrate that the present system is capable of producing a high yield of active NADase, NADase-Flag fusions were cloned into the backbone of an illustrative system (EG13).

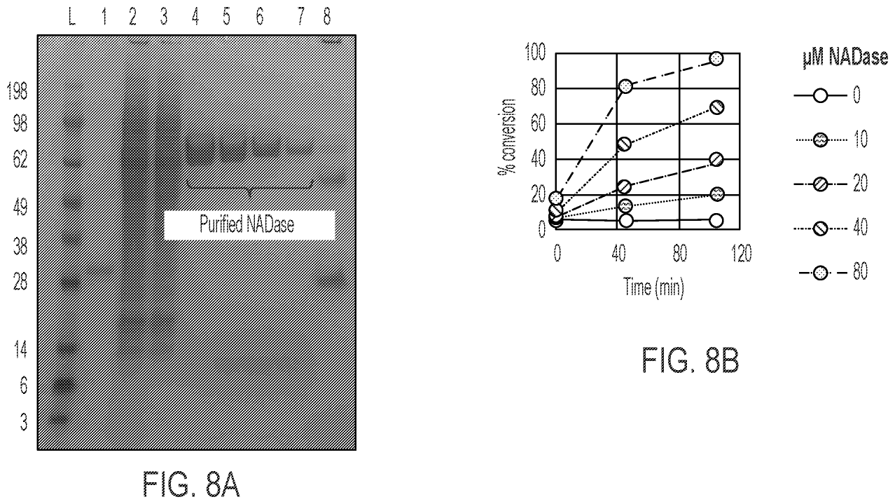

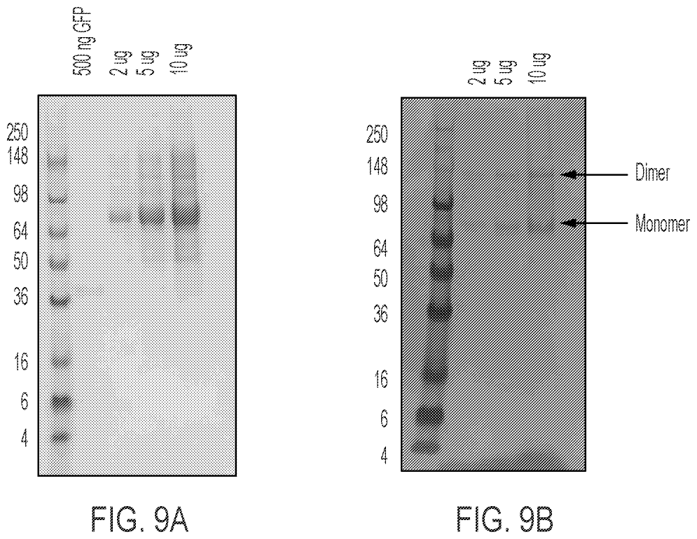

NADase-flag construct was expressed in HEK293 cells. HEK293 cells were seeded at 5×10⁶ cells in a T225 flask and incubated at 37° C. and 5% CO₂ overnight before transiently transfecting with either EG13 as described above. NADase-flag expression was monitored after 24 h and 48 h using microscopy. Cells were harvested after 48 h by detaching the cells using 0.5% trypsin solution for 5 min at 37° C. and scraping. Cells were pelleted (5,000×g, 15 min, 4° C.) and supernatant was discarded. Cell pellets were stored at −80° C. until further usage. To purify NADase-flag, cells were resuspended in lysis buffer (50 mM NaHPO4 pH 8.0, 300 mM NaCl, 0.01% Tween20, protease inhibitor, DNAse) and lysed by sonication (2 min, 10 s ON, 10 s OFF, 40% Amplitude) and crude cell extract was cleared (100,000×g, 45 min, 4° C.). ANTI-FLAG M2 Affinity Gel (Sigma) was equilibrated with wash buffer (50 mM NaHPO4 pH 8.0, 300 mM NaCl, 0.01% Tween20) prior to adding to the cleared lysate. Lysate was incubated with the resin for 2 h at 4° C. with shaking. Resin was settled and washed with 5 CV wash buffer and proteins was eluted with 4× 1 CV elution buffer (wash buffer+0.2 mg/ml 3× Flag-peptide (Sigma)) using spin columns. Purification was analyzed by SDS-PAGE (6-12% BOLT, ThermoFisher) (FIG. 8A) and protein containing fractions were pooled. Protein concentration was measured using A280 (NanoDrop One, FisherScientific). Protein yields were determined to be 26 mg/L expression medium. The activity of NADase was tested by analyzing the conversion rate of NAD+ to ADP-ribose by HPLC (FIG. 8B).

### Example 13: Production of a Secreted Protein, C1 Esterase Inhibitor (C1-Inh)

C1-Inh was used as an illustrative target protein to exemplify the application of the disclosed methods for expressing secreted proteins with the correct post-translational modifications. C1-Inh is a protease inhibitor belonging to the serpin superfamily. As a secreted protein C1-Inh is highly glycosylated and therefore proves to be a difficult target for recombinant expression. C1-Inh-myc-flag fusion protein was expressed in the presence or absence of the L protein from EMCV which was expressed from a separate construct. In this example, the L-protein from EMCV was co-expressed from a separate construct under control of a CMV promoter.

C1-Inh-Myc-Flag fusions were expressed in HEK293 cells. HEK293 cells were seeded at 1.75×10⁶/ml cells in 100 ml shaking flask and incubated at 37° C., 5% CO₂ and 120 rpm overnight before transiently transfecting with a vector encoding C1-Inh (OriGene; CAT #: RC203767) either alone, or in combination with EG11 by transfection of suspension cells using methods known in the art and/or disclosed herein. Supernatant containing the expressed recombinant C1-Inh protein was harvested after 72 h and supernatant was cleared by centrifugation followed by filtration (22 um, nitrocellulose). To purify C1-Inh, Anti-Flag resin (ANTI-FLAG M2 Affinity Gel, Millipore Sigma) was equilibrated with 20 mM Tris pH 7.5, 50 mM NaCl prior to adding to the supernatant. Supernatant was incubated with the resin for 2 h at 4° C. with shaking. Resin was settled and washed with 5 CV 20 mM Tris pH 7.5, 50 mM NaCl and protein was eluted with 4 CV 20 mM Tris pH 7.5, 50 mM NaCl, 0.2 mg/ml 3× Flag Peptide. Purification was analyzed by SDS-PAGE (SurePAGE, Bis-Tris, GenScript) and protein containing fractions were pooled. Protein concentration was analyzed by BCA Assay (ThermoFisher) according to manufactures instructions and normalized C1-Inh was tested for activity using Immunoassay (MicroVue C1-Inhibitor Plus EIA, Quidel) following manufactures instructions.

FIG. 20A shows the purification of C1-Inhibitor in absence (left) and presence (right) of an enhancer protein. The total amount of produced C1-Inhibitor is increased by >30% in the presence of the enhancer protein. FIG. 20B demonstrates the improvement of the total amount of active C1-Inhibitor within the purified sample. For the activity assay, the protein concentration was normalized before testing for active C1-Inhibitor. The amount of active C1-Inhibitor could be increased by >10% by co-expressing the enhancer protein simultaneously with the GOI. These results demonstrate that the methods disclosed herein result in higher yields and improved activity of secreted target proteins, such as C1-Inhibitor.

### Example 14: Production of a Secreted Protein, Pregnancy Specific Glycoprotein 1 (PSG1)

PSG1 was used as an illustrative target protein to exemplify the application of the disclosed methods for expressing secreted proteins with the correct post-translational modifications. PSG1 is a highly glycosylated secreted protein of the human PSG family within the carcinoembryonic antigen superfamily. PSG1 is one of the most abundant fetal proteins found in maternal blood during pregnancy. PSG1 has been shown to serve as an immunomodulator by up-regulating of TGF-beta in macrophages, monocytes, and trophoblasts. In addition, PSG1 has been shown to induce secretion of anti-inflammatory cytokines IL-10 and IL-6 in human monocytes. These functions made PSG1 an attractive pharmaceutical target. The difficulty while expressing PSG1, is the right glycosylation pattern that is impossible to recreate while using non-human cells. In this example, the L-protein from EMCV was co-expressed with PSG1 under control of a CMV promoter.

PSG1 were expressed in HEK293 cells. HEK293 cells were seeded at 1.75×10⁶/ml cells in 100 ml shaking flask and incubated at 37° C., 5% CO₂ and 120 rpm overnight before transiently transfecting with a vector encoding PSG1 in tandem with the L-protein from EMCV. Supernatant containing the expressed recombinant PSG1 protein was harvested after 72 h and supernatant was cleared by centrifugation followed by filtration (22 um, nitrocellulose). To purify PSG1, HiTrap™ DEAE Sepharose Fast Flow IEX Columns (Cytiva (Formerly GE Healthcare Life Sciences) was equilibrated with wash buffer (10 mM Tris pH 7.6) prior to loading the column with the supernatant using a peristaltic pump. After loading, the purification was performed on an ÄKTA™ system (Cytiva Life Sciences (former GE Healthcare)). The column was washed with 5CV wash buffer before eluting with a multi-step gradient 10%, 20%, 30%, 50% and 100% elution buffer (wash buffer+200 mM NaCl). Protein containing fraction were pooled, concentrated and analyzed by SDS-PAGE (6-12% BOLT, ThermoFisher) and Western blot (Nitrocellulose membrane, ThermoFisher) using anti-PSG1 (Invitrogen, 2^{nd} antibody-anti-rabbit-HRP).

FIG. 21 shows the ion exchange chromatography of PSG1 (left). Protein containing fractions (FIG. 21A, red box) were pooled and concentrated before confirming the presence and identity of PSG1 by SDS-PAGE and Western blot (FIG. 21B, red arrow).

### Further Numbered Embodiments

Further embodiments of the instant invention are provided in the numbered embodiments below:
Embodiment 1. A system for recombinant expression of a target protein in eukaryotic cells, comprising one or more vectors, the one or more vectors comprising:
   a. a first polynucleotide encoding the target protein; and
   b. a second polynucleotide encoding an enhancer protein wherein:
      i. the enhancer protein is an inhibitor of nucleocytoplasmic transport (NCT) and/or
      ii. the enhancer protein is selected from the group consisting of a picornavirus leader (L) protein, a picornavirus 2A protease, a rhinovirus 3C protease, a herpes simplex virus (HSV) ICP27 protein, and a rhabdovirus matrix (M) protein,
   wherein the first polynucleotide and the second polynucleotide are operatively linked to one or more promoters.
Embodiment 2. The system of embodiment 1, wherein the enhancer protein is an inhibitor of nucleocytoplasmic transport (NCT).
Embodiment 3. The system of embodiment 2, wherein the NCT inhibitor is a viral protein.
Embodiment 4. The system of any one of embodiments 1 to 3, wherein the NCT inhibitor is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.
Embodiment 5. The system of embodiment 4, wherein the NCT inhibitor is a picornavirus leader (L) protein or a functional variant thereof.
Embodiment 6. The system of embodiment 4, wherein the NCT inhibitor is a picornavirus 2A protease or a functional variant thereof.
Embodiment 7. The system of embodiment 4, wherein the NCT inhibitor is a rhinovirus 3C protease or a functional variant thereof.
Embodiment 8. The system of embodiment 4, wherein the NCT inhibitor is a coronavirus ORF6 protein or a functional variant thereof.
Embodiment 9. The system of embodiment 4, wherein the NCT inhibitor is an ebolavirus VP24 protein or a functional variant thereof.
Embodiment 10. The system of embodiment 4, wherein the NCT inhibitor is a

Venezuelan equine encephalitis virus (VEEV) capsid protein or a functional variant thereof.
Embodiment 11. The system of embodiment 4, wherein the NCT inhibitor is a herpes simplex virus (HSV) ICP27 protein or a functional variant thereof.
Embodiment 12. The system of embodiment 4, wherein the NCT inhibitor is a rhabdovirus matrix (M) protein or a functional variant thereof.
Embodiment 13. The system of embodiment 5, wherein the L protein is the L protein of

Theiler's virus or a functional variant thereof.
Embodiment 14. The system of embodiment 5, wherein the L protein shares at least 90% identity to SEQ ID NO: 1.
Embodiment 15. The system of embodiment 5, wherein the L protein is the L protein of Encephalomyocarditis virus (EMCV) or a functional variant thereof.
Embodiment 16. The system of embodiment 5, wherein the L protein shares at least 90% identity to SEQ ID NO: 2.
Embodiment 17. The system of embodiment 5, wherein the L protein is selected from the group consisting of the L protein of poliovirus, the L protein of HRV16, the L protein of mengo virus, and the L protein of Saffold virus 2 or a functional variant thereof.
Embodiment 18. The system of any one of embodiments 1 to 17, wherein the system comprises a single vector comprising an expression cassette, the expression cassette comprising the first polynucleotide and the second polynucleotide.
Embodiment 19. The system of embodiment 18, wherein the expression cassette comprises a first promoter, operatively linked to the first polynucleotide; and a second promoter, operatively linked to the second polynucleotide.
Embodiment 20. The system of embodiment 18, wherein the expression cassette comprises a shared promoter operatively linked to both the first polynucleotide and the second polynucleotide.
Embodiment 21. The system of embodiment 20, wherein the expression cassette comprises a coding polynucleotide comprising the first polynucleotide and the second polynucleotide linked by a polynucleotide encoding ribosome skipping site, the coding polynucleotide operatively linked to the shared promoter.
Embodiment 22. The system of embodiment 20, wherein the expression cassette comprises a coding polynucleotide, the coding polynucleotide encoding the enhancer protein and the target protein linked to by a ribosome skipping site, the coding polynucleotide operatively linked to the shared promoter.
Embodiment 23. The system of any one of embodiments 18 to 22, wherein the expression cassette is configured for transcription of a single messenger RNA encoding both the target protein and the enhancer protein, linked by a ribosome skipping site; wherein translation of the messenger RNA results in expression of the target protein and the L protein as distinct polypeptides.
Embodiment 24. The system of any one of embodiments 1 to 23, wherein the system comprises one vector.
Embodiment 25. The system of any one of embodiments 1 to 17, wherein the system comprises:
   a. a first vector comprising the first polynucleotide, operatively linked to a first promoter; and
   b. a second vector comprising the second polynucleotide, operatively linked to a second promoter.
Embodiment 26. The system of any one of embodiments 1 to 17 or embodiment 25, wherein the system comprises two vectors.
Embodiment 27. The system of any one of embodiments 1 to 26, wherein either the first polynucleotide or the second polynucleotide, or both, are operatively linked to an internal ribosome entry site (IRES).
Embodiment 28. The system of any one of embodiments 1 to 27, wherein at least one of the one or more vectors comprises a T7 promoter configured for transcription of either or both of the first polynucleotide and the second polynucleotide by a T7 RNA polymerase.
Embodiment 29. The system of any one of embodiments 1 to 28, wherein at least one of the one or more vectors comprises a polynucleotide sequence encoding a T7 RNA polymerase.
Embodiment 30. A vector for recombinant expression of a target protein in eukaryotic cells, comprising:
   a. a first polynucleotide encoding the target protein; and
   b. a second polynucleotide encoding an enhancer protein wherein:
      i. the enhancer protein is an inhibitor of nucleocytoplasmic transport (NCT) and/or
      ii. the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.
   wherein the first polynucleotide and the second polynucleotide are operatively linked to at least one promoter.
Embodiment 31. The vector of embodiment 30, wherein the expression cassette comprises a first promoter, operatively linked to the first polynucleotide; and a second promoter, operatively linked to the second polynucleotide.
Embodiment 32. The vector of embodiment 30, wherein the expression cassette comprises a shared promoter operatively linked to both the first polynucleotide and the second polynucleotide.
Embodiment 33. A eukaryotic cell for expression of a target protein, comprising an exogenous polynucleotide encoding an enhancer protein wherein:
   a. the enhancer protein is an inhibitor of nucleocytoplasmic transport (NCT) and/or
   b. the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein,
   wherein the exogenous polynucleotide is operatively linked to a promoter
Embodiment 34. The eukaryotic cell of embodiment 33, wherein the polynucleotide is operatively linked to an internal ribosome entry site (IRES).
Embodiment 35. The eukaryotic cell of embodiment 33 or embodiment 34, wherein the promoter is an inducible promoter.
Embodiment 36. A method for recombinant expression of a target protein, comprising introducing a polynucleotide encoding the target protein, operatively linked to a promoter, into the cell of any one of embodiments 33 to 35.
Embodiment 37. A method for recombinant expression of a target protein, comprising introducing the system of any one of embodiments 1 to 29 or the vector of any one of embodiments 30 to 32 into eukaryotic cell.
Embodiment 38. The method of embodiment 36 or embodiment 37, wherein the target protein is a membrane protein
Embodiment 39. The method of any embodiment 38, wherein localization of the membrane protein to the cellular membrane is increased compared to the localization observed when the membrane protein is expressed without the enhancer protein.
Embodiment 40. A eukaryotic cell produced by introduction of the system of any one of embodiments 1 to 29, or the vector of any one of embodiments 30 to 32 into the eukaryotic cell.
Embodiment 41. A target protein expressed by introduction of the system of any one of embodiments 1 to 29 or the vector of any one of embodiments 30 to 32 into a eukaryotic cell.
Embodiment 42. A method for expressing a target protein in eukaryotic cells, comprising introducing a polynucleotide encoding the target protein, the polynucleotide operatively linked to a promoter, into the eukaryotic cells, wherein the method utilizes co-expression of an enhancer protein to enhance the expression level, solubility and/or activity of the target protein, wherein: (a) the enhancer protein is an inhibitor of nucleocytoplasmic transport (NCT) and/or (b) the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.
Embodiment 43. The method of embodiment 42, wherein the co-expression of enhancer protein comprises introducing into the eukaryotic cell a polynucleotide encoding the enhancer protein, operatively linked to a promoter.
Embodiment 44. The method of embodiment 42 or embodiment 43, wherein the introducing step or steps comprise transfection of the eukaryotic cells with one or more DNA molecules, transduction of the eukaryotic cells with a single viral vector, and/or transduction of the eukaryotic cells with two viral vectors.
Embodiment 45. The system of any one of embodiments 1 to 29, the vector of any one of embodiments 30 to 32, the eukaryotic cell of any one of embodiments 33 to 35, the method of any one of embodiments 36 to 39 and 42-44, the eukaryotic cell of embodiment 40, and the target protein of embodiment 41, wherein the target protein is a soluble protein.
Embodiment 46. The system of any one of embodiments 1 to 29, the vector of any one of embodiments 30 to 32, the cell of any one of embodiments 33 to 35, or the method of any one of embodiments 36 to 44, wherein the target protein is a secreted protein.
Embodiment 47. The system of any one of embodiments 1 to 29, the vector of any one of embodiments 30 to 32, the eukaryotic cell of any one of embodiments 33 to 35, the method of any one of embodiments 36 to 39 and 42-44, the eukaryotic cell of embodiment 40, and the target protein of embodiment 41, wherein the target protein is a membrane protein.
Embodiment 48. The system of any one of embodiments 1 to 29, the vector of any one of embodiments 30 to 32, the eukaryotic cell of any one of embodiments 33 to 35, the method of any one of embodiments 36 to 39 and 42-44, the eukaryotic cell of embodiment 40, and the target protein of embodiment 41, wherein the target protein is Dopamine receptor 1 (DRD1), optionally wherein the DRD1 comprises an amino acid sequence having at least 90% identity to the amino acid sequence of SEQ ID NO: 19.
Embodiment 49. The system of any one of embodiments 1 to 29, the vector of any one of embodiments 30 to 32, the eukaryotic cell of any one of embodiments 33 to 35, the method of any one of embodiments 36 to 39 and 42-44, the eukaryotic cell of embodiment 40, and the target protein of embodiment 41, wherein the target protein is Cystic fibrosis transmembrane conductance regulator (CFTR), optionally wherein the CFTR comprises an amino acid sequence having at least 90% identity to the amino acid sequence of SEQ ID NO: 18.
Embodiment 50. The system of any one of embodiments 1 to 29, the vector of any one of embodiments 30 to 32, the eukaryotic cell of any one of embodiments 33 to 35, the method of any one of embodiments 36 to 39 and 42-44, the eukaryotic cell of embodiment 40, and the target protein of embodiment 41, wherein the target protein is Cl esterase inhibitor (C1-Inh), optionally wherein the C1-Inh comprises an amino acid sequence having at least 90% identity to the amino acid sequence of SEQ ID NO: 16.
Embodiment 51. The system of any one of embodiments 1 to 29, the vector of any one of embodiments 30 to 32, the eukaryotic cell of any one of embodiments 33 to 35, the method of any one of embodiments 36 to 39 and 42-44, the eukaryotic cell of embodiment 40, and the target protein of embodiment 41, wherein the target protein is ITK, optionally wherein the ITK comprises an amino acid sequence having at least 90% identity to the amino acid sequence of SEQ ID NO: 15.
Embodiment 52. The system of any one of embodiments 1 to 29, the vector of any one of embodiments 30 to 32, the eukaryotic cell of any one of embodiments 33 to 35, the method of any one of embodiments 36 to 39 and 42-44, the eukaryotic cell of embodiment 40, and the target protein of embodiment 41, wherein the target protein is an NADase, optionally wherein the NADase comprises an amino acid sequence having at least 90% identity to the amino acid sequence of SEQ ID NO: 20.
Embodiment 53. A method for generating an antibody against a target protein, comprising immunizing a subject with the cell of any one of embodiments 33 to 35, the cell of embodiment 40, or the target protein of embodiment 41.
Embodiment 54. The method of embodiment 53, further comprising isolating one or more immune cells expressing an immunoglobulin protein specific for the target protein.
Embodiment 55. The method of embodiment 53 or embodiment 54, comprising generating one or more hybridomas from the one or more immune cells.
Embodiment 56. The method of any one of embodiments 53 to 55, comprising cloning one or more immunoglobulin genes from the one or more immune cells.
Embodiment 57. A method for antibody discovery by cell sorting, comprising providing a solution comprising:
   a. the cell of any one of embodiments 33 to 35, the eukaryotic cell of embodiment 40, or the target protein of embodiment 41, wherein the cell or target protein is labeled, and
   b. a population of recombinant cells, wherein the recombinant cells express a library of polypeptides each comprising an antibody or antigen-binding fragment thereof; and isolating one or more recombinant cells from the solution by sorting for recombinant cells bound to the labeled cell or the labeled target protein.
Embodiment 58. A method for panning a phage-display library, comprising:
   a. mixing a phage-display library with the eukaryotic cell of any one of embodiments 33 to 35, the eukaryotic cell of embodiment 40, or the target protein of embodiment 41; and
   b. purifying and/or enriching the members of the phage-display library that bind the cell or target protein.
Embodiment 59. The eukaryotic cell of any one of embodiments 33-35 and 40, wherein the eukaryotic cell is a human cell, an animal cell, an insect cell, a plant cell, or a fungal cell.
Embodiment 60. The eukaryotic cell of any one of embodiments 33-35, 40, and 59, wherein the eukaryotic cell is a eukaryotic cell line.
Embodiment 61. The eukaryotic cell of any one of embodiments 33-35, 40, 59 and 60, wherein the eukaryotic cell is Bc HROC277, COS, CHO, CHO-S, CHO-K1, CHO-DG44, CHO-DUXB11, CHO-DUKX, CHOK1SV, VERO, MDCK, W138, V79, B14AF28-G3, BHK, HaK, NSO, 5P2/0-Ag14, HeLa, HEK293, HEK293-F, HEK293-H, HEK293-T, perC6 cell, Sf9 cell, a Saccharomyces cell, a Pichia cell or a Schizosaccharomyces; cell.
Embodiment 62. The eukaryotic cell of embodiment 60, wherein the eukaryotic cell line is a stable cell line.
Embodiment 63. The system of any one of embodiments 1-29 and 45-52, wherein the one or more vectors is selected from the group consisting of adeno-associated virus (AAV) vector, a lentivirus vector, a retrovirus vector, a replication competent adenovirus vector, a replication deficient adenovirus vector, a herpes virus vector, a baculovirus vector or a non-viral plasmid.
Embodiment 64. The system of embodiment 63, wherein at least one of the one or more vectors is an AAV vector.
Embodiment 65. The vector of any one of embodiments 30-32, wherein the vector is an adeno-associated virus (AAV) vector, a lentivirus vector, a retrovirus vector, a replication competent adenovirus vector, a replication deficient adenovirus vector, a herpes virus vector, a baculovirus vector or a non-viral plasmid.
Embodiment 66. The vector of embodiment 65, wherein the vector is an AAV vector.
Embodiment 67. The system of embodiment 4, wherein the rhabdovirus matrix (M) protein is a M protein of Vesicular stomatitis virus (VSV).
Embodiment 68. The system of embodiment 67, wherein the M protein shares at least 90% identity to SEQ ID NO: 9.
Embodiment 69. A system for recombinant expression of a target protein in eukaryotic cells, comprising one or more vectors, the one or more vectors comprising:
   a. a first polynucleotide encoding the target protein; and
   b. a second polynucleotide encoding an L protein of Encephalomyocarditis virus (EMCV), optionally wherein the L protein shares at least 90% identity to SEQ ID NO: 2, and
   wherein the first polynucleotide and the second polynucleotide are operatively linked to one or more promoters.
Embodiment 70. A system for recombinant expression of a target protein in eukaryotic cells, comprising one or more vectors, the one or more vectors comprising:
   a. a first polynucleotide encoding the target protein; and
   b. a second polynucleotide encoding a L protein of Theiler's virus, optionally wherein the L protein shares at least 90% identity to SEQ ID NO: 1, and
   wherein the first polynucleotide and the second polynucleotide are operatively linked to one or more promoters.
Embodiment 71. A system for recombinant expression of a target protein in eukaryotic cells, comprising one or more vectors, the one or more vectors comprising:
   a. a first polynucleotide encoding the target protein; and
   b. a second polynucleotide encoding a picornavirus 2A protease, optionally wherein the picornavirus 2A protease shares at least 90% identity to SEQ ID NO: 7, and
   wherein the first polynucleotide and the second polynucleotide are operatively linked to one or more promoters.
Embodiment 72. A system for recombinant expression of a target protein in eukaryotic cells, comprising one or more vectors, the one or more vectors comprising:
   a. a first polynucleotide encoding the target protein; and
   b. a second polynucleotide encoding a M protein of Vesicular stomatitis virus (VSV), optionally wherein the M protein shares at least 90% identity to SEQ ID NO: 9, and
   wherein the first polynucleotide and the second polynucleotide are operatively linked to one or more promoters.
Embodiment 73. The system of any one of embodiments 69-72, wherein the target protein is Dopamine receptor 1 (DRD1), optionally wherein the DRD1 comprises an amino acid sequence having at least 90% identity to the amino acid sequence of SEQ ID NO: 19.
Embodiment 74. The system of any one of embodiments 69-72, wherein the target protein is Cystic fibrosis transmembrane conductance regulator (CFTR), optionally wherein the CFTR comprises an amino acid sequence having at least 90% identity to the amino acid sequence of SEQ ID NO: 18.
Embodiment 75. The system of any one of embodiments 69-72, wherein the target protein is Cl esterase inhibitor (C1-Inh), optionally wherein the C1-Inh comprises an amino acid sequence having at least 90% identity to the amino acid sequence of SEQ ID NO: 16.
Embodiment 76. The system of any one of embodiments 69-72, wherein the target protein is ITK, optionally wherein the ITK comprises an amino acid sequence having at least 90% identity to the amino acid sequence of SEQ ID NO: 15.
Embodiment 77. The system of any one of embodiments 69-72, wherein the target protein is an NADase, optionally wherein the NADase comprises an amino acid sequence having at least 90% identity to the amino acid sequence of SEQ ID NO: 20.

What is claimed is:

1. A system for recombinant expression of a target protein in eukaryotic cells, comprising one or more vectors, the one or more vectors comprising:
a) a first polynucleotide encoding the target protein; and
b) a second polynucleotide encoding an enhancer protein wherein:
i) the enhancer protein is an inhibitor of nucleocytoplasmic transport (NCT) and/or
ii) the enhancer protein is selected from the group consisting of a picornavirus leader (L) protein, a picornavirus 2A protease, a rhinovirus 3C protease, a herpes simplex virus (HSV) ICP27 protein, and a rhabdovirus matrix (M) protein,
wherein the first polynucleotide and the second polynucleotide are operatively linked to one or more promoters.

2. The system of claim 1, wherein the enhancer protein is an inhibitor of nucleocytoplasmic transport (NCT).

3. The system of claim 2, wherein the NCT inhibitor is a viral protein.

4. The system of any one of claims 1 to 3, wherein the NCT inhibitor is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

5. The system of claim 4, wherein the NCT inhibitor is a picornavirus leader (L) protein or a functional variant thereof.

6. The system of claim 4, wherein the NCT inhibitor is a picornavirus 2A protease or a functional variant thereof.

7. The system of claim 4, wherein the NCT inhibitor is a rhinovirus 3C protease or a functional variant thereof.

8. The system of claim 4, wherein the NCT inhibitor is a coronavirus ORF6 protein or a functional variant thereof.

9. The system of claim 4, wherein the NCT inhibitor is an ebolavirus VP24 protein or a functional variant thereof.

10. The system of claim 4, wherein the NCT inhibitor is a Venezuelan equine encephalitis virus (VEEV) capsid protein or a functional variant thereof.

11. The system of claim 4, wherein the NCT inhibitor is a herpes simplex virus (HSV) ICP27 protein or a functional variant thereof.

12. The system of claim 4, wherein the NCT inhibitor is a rhabdovirus matrix (M) protein or a functional variant thereof.

13. The system of claim 5, wherein the L protein is the L protein of Theiler's virus or a functional variant thereof.

14. The system of claim 5, wherein the L protein shares at least 90% identity to SEQ ID NO: 1.

15. The system of claim 5, wherein the L protein is the L protein of Encephalomyocarditis virus (EMCV) or a functional variant thereof.

16. The system of claim 5, wherein the L protein shares at least 90% identity to SEQ ID NO: 2.

17. The system of claim 5, wherein the L protein is selected from the group consisting of the L protein of poliovirus, the L protein of HRV16, the L protein of mengo virus, and the L protein of Saffold virus 2 or a functional variant thereof.

18. The system of any one of claims 1 to 17, wherein the system comprises a single vector comprising an expression cassette, the expression cassette comprising the first polynucleotide and the second polynucleotide.

19. The system of claim 18, wherein the expression cassette comprises a first promoter, operatively linked to the first polynucleotide; and a second promoter, operatively linked to the second polynucleotide.

20. The system of claim 18, wherein the expression cassette comprises a shared promoter operatively linked to both the first polynucleotide and the second polynucleotide.

21. The system of claim 20, wherein the expression cassette comprises a coding polynucleotide comprising the first polynucleotide and the second polynucleotide linked by a polynucleotide encoding ribosome skipping site, the coding polynucleotide operatively linked to the shared promoter.

22. The system of claim 20, wherein the expression cassette comprises a coding polynucleotide, the coding polynucleotide encoding the enhancer protein and the target protein linked to by a ribosome skipping site, the coding polynucleotide operatively linked to the shared promoter.

23. The system of any one of claims 18 to 22, wherein the expression cassette is configured for transcription of a single messenger RNA encoding both the target protein and the enhancer protein, linked by a ribosome skipping site; wherein translation of the messenger RNA results in expression of the target protein and the L protein as distinct polypeptides.

24. The system of any one of claims 1 to 23, wherein the system comprises one vector.

25. The system of any one of claims 1 to 17, wherein the system comprises:
a) a first vector comprising the first polynucleotide, operatively linked to a first promoter; and
b) a second vector comprising the second polynucleotide, operatively linked to a second promoter.

26. The system of any one of claims 1 to 17 or claim 25, wherein the system comprises two vectors.

27. The system of any one of claims 1 to 26, wherein either the first polynucleotide or the second polynucleotide, or both, are operatively linked to an internal ribosome entry site (IRES).

28. The system of any one of claims 1 to 27, wherein at least one of the one or more vectors comprises a T7 promoter configured for transcription of either or both of the first polynucleotide and the second polynucleotide by a T7 RNA polymerase.

29. The system of any one of claims 1 to 28, wherein at least one of the one or more vectors comprises a polynucleotide sequence encoding a T7 RNA polymerase.

30. A vector for recombinant expression of a target protein in eukaryotic cells, comprising:
a) a first polynucleotide encoding the target protein; and
b) a second polynucleotide encoding an enhancer protein wherein:
i) the enhancer protein is an inhibitor of nucleocytoplasmic transport (NCT) and/or
ii) the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.
wherein the first polynucleotide and the second polynucleotide are operatively linked to at least one promoter.

31. The vector of claim 30, wherein the expression cassette comprises a first promoter, operatively linked to the first polynucleotide; and a second promoter, operatively linked to the second polynucleotide.

32. The vector of claim 30, wherein the expression cassette comprises a shared promoter operatively linked to both the first polynucleotide and the second polynucleotide.

33. A eukaryotic cell for expression of a target protein, comprising an exogenous polynucleotide encoding an enhancer protein wherein:
a) the enhancer protein is an inhibitor of nucleocytoplasmic transport (NCT) and/or
b) the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein,
wherein the exogenous polynucleotide is operatively linked to a promoter

34. The eukaryotic cell of claim 33, wherein the polynucleotide is operatively linked to an internal ribosome entry site (IRES).

35. The eukaryotic cell of claim 33 or claim 34, wherein the promoter is an inducible promoter.

36. A method for recombinant expression of a target protein, comprising introducing a polynucleotide encoding the target protein, operatively linked to a promoter, into the cell of any one of claims 33 to 35.

37. A method for recombinant expression of a target protein, comprising introducing the system of any one of claims 1 to 29 or the vector of any one of claims 30 to 32 into eukaryotic cell.

38. The method of claim 36 or claim 37, wherein the target protein is a membrane protein

39. The method of any claim 38, wherein localization of the membrane protein to the cellular membrane is increased compared to the localization observed when the membrane protein is expressed without the enhancer protein.

40. A eukaryotic cell produced by introduction of the system of any one of claims 1 to 29, or the vector of any one of claims 30 to 32 into the eukaryotic cell.

41. A target protein expressed by introduction of the system of any one of claims 1 to 29 or the vector of any one of claims 30 to 32 into a eukaryotic cell.

42. A method for expressing a target protein in eukaryotic cells, comprising introducing a polynucleotide encoding the target protein, the polynucleotide operatively linked to a promoter, into the eukaryotic cells,
wherein the method utilizes co-expression of an enhancer protein to enhance the expression level, solubility and/or activity of the target protein,
wherein:
a) the enhancer protein is an inhibitor of nucleocytoplasmic transport (NCT) and/or
b) the enhancer protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

43. The method of claim 42, wherein the co-expression of enhancer protein comprises introducing into the eukaryotic cell a polynucleotide encoding the enhancer protein, operatively linked to a promoter.

44. The method of claim 42 or claim 43, wherein the introducing step or steps comprise transfection of the eukaryotic cells with one or more DNA molecules, transduction of the eukaryotic cells with a single viral vector, and/or transduction of the eukaryotic cells with two viral vectors.

45. The system of any one of claims 1 to 29, the vector of any one of claims 30 to 32, the eukaryotic cell of any one of claims 33 to 35, the method of any one of claims 36 to 39 and 42-44, the eukaryotic cell of claim 40, and the target protein of claim 41, wherein the target protein is a soluble protein.

46. The system of any one of claims 1 to 29, the vector of any one of claims 30 to 32, the cell of any one of claims 33 to 35, or the method of any one of claims 36 to 44, wherein the target protein is a secreted protein.

47. The system of any one of claims 1 to 29, the vector of any one of claims 30 to 32, the eukaryotic cell of any one of claims 33 to 35, the method of any one of claims 36 to 39 and 42-44, the eukaryotic cell of claim 40, and the target protein of claim 41, wherein the target protein is a membrane protein.

48. The system of any one of claims 1 to 29, the vector of any one of claims 30 to 32, the eukaryotic cell of any one of claims 33 to 35, the method of any one of claims 36 to 39 and 42-44, the eukaryotic cell of claim 40, and the target protein of claim 41, wherein the target protein is Dopamine receptor 1 (DRD1), optionally wherein the DRD1 comprises an amino acid sequence having at least 90% identity to the amino acid sequence of SEQ ID NO: 19.

49. The system of any one of claims 1 to 29, the vector of any one of claims 30 to 32, the eukaryotic cell of any one of claims 33 to 35, the method of any one of claims 36 to 39 and 42-44, the eukaryotic cell of claim 40, and the target protein of claim 41, wherein the target protein is Cystic fibrosis transmembrane conductance regulator (CFTR), optionally wherein the CFTR comprises an amino acid sequence having at least 90% identity to the amino acid sequence of SEQ ID NO: 18.

50. The system of any one of claims 1 to 29, the vector of any one of claims 30 to 32, the eukaryotic cell of any one of claims 33 to 35, the method of any one of claims 36 to 39 and 42-44, the eukaryotic cell of claim 40, and the target protein of claim 41, wherein the target protein is C1 esterase inhibitor (C1-Inh), optionally wherein the C1-Inh comprises an amino acid sequence having at least 90% identity to the amino acid sequence of SEQ ID NO: 16.

51. The system of any one of claims 1 to 29, the vector of any one of claims 30 to 32, the eukaryotic cell of any one of claims 33 to 35, the method of any one of claims 36 to 39 and 42-44, the eukaryotic cell of claim 40, and the target protein of claim 41, wherein the target protein is ITK, optionally wherein the ITK comprises an amino acid sequence having at least 90% identity to the amino acid sequence of SEQ ID NO: 15.

52. The system of any one of claims 1 to 29, the vector of any one of claims 30 to 32, the eukaryotic cell of any one of claims 33 to 35, the method of any one of claims 36 to 39 and 42-44, the eukaryotic cell of claim 40, and the target protein of claim 41, wherein the target protein is an NADase, optionally wherein the NADase comprises an amino acid sequence having at least 90% identity to the amino acid sequence of SEQ ID NO: 20.

53. A method for generating an antibody against a target protein, comprising immunizing a subject with the cell of any one of claims 33 to 35, the cell of claim 40, or the target protein of claim 41.

54. The method of claim 53, further comprising isolating one or more immune cells expressing an immunoglobulin protein specific for the target protein.

55. The method of claim 53 or claim 54, comprising generating one or more hybridomas from the one or more immune cells.

56. The method of any one of claims 53 to 55, comprising cloning one or more immunoglobulin genes from the one or more immune cells.

57. A method for antibody discovery by cell sorting, comprising providing a solution comprising:
a) the cell of any one of claims 33 to 35, the eukaryotic cell of claim 40, or the target protein of claim 41, wherein the cell or target protein is labeled, and
b) a population of recombinant cells, wherein the recombinant cells express a library of polypeptides each comprising an antibody or antigen-binding fragment thereof; and
isolating one or more recombinant cells from the solution by sorting for recombinant cells bound to the labeled cell or the labeled target protein.

58. A method for panning a phage-display library, comprising:
a) mixing a phage-display library with the eukaryotic cell of any one of claims 33 to 35, the eukaryotic cell of claim 40, or the target protein of claim 41; and
b) purifying and/or enriching the members of the phage-display library that bind the cell or target protein.

59. The eukaryotic cell of any one of claims 33-35 and 40, wherein the eukaryotic cell is a human cell, an animal cell, an insect cell, a plant cell, or a fungal cell.

60. The eukaryotic cell of any one of claims 33-35, 40, and 59, wherein the eukaryotic cell is a eukaryotic cell line.

61. The eukaryotic cell of any one of claims 33-35, 40, 59 and 60, wherein the eukaryotic cell is Bc HROC277, COS, CHO, CHO-S, CHO-K1, CHO-DG44, CHO-DUXB11, CHO-DUKX, CHOK1SV, VERO, MDCK, WI38, V79, B14AF28-G3, BHK, HaK, NSO, 5P2/0-Ag14, HeLa, HEK293, HEK293-F, HEK293-H, HEK293-T, perC6 cell, Sf9 cell, a Saccharomyces cell, a Pichia cell or a Schizosaccharomyces cell.

62. The eukaryotic cell of claim 60, wherein the eukaryotic cell line is a stable cell line.

63. The system of any one of claims 1-29 and 45-52, wherein the one or more vectors is selected from the group consisting of adeno-associated virus (AAV) vector, a lentivirus vector, a retrovirus vector, a replication competent adenovirus vector, a replication deficient adenovirus vector, a herpes virus vector, a baculovirus vector or a non-viral plasmid.

64. The system of claim 63, wherein at least one of the one or more vectors is an AAV vector.

65. The vector of any one of claims 30-32, wherein the vector is an adeno-associated virus (AAV) vector, a lentivirus vector, a retrovirus vector, a replication competent adenovirus vector, a replication deficient adenovirus vector, a herpes virus vector, a baculovirus vector or a non-viral plasmid.

66. The vector of claim 65, wherein the vector is an AAV vector.

67. The system of claim 4, wherein the rhabdovirus matrix (M) protein is a M protein of Vesicular stomatitis virus (VSV).

68. The system of claim 67, wherein the M protein shares at least 90% identity to SEQ ID NO: 9.

69. A system for recombinant expression of a target protein in eukaryotic cells, comprising one or more vectors, the one or more vectors comprising:
a) a first polynucleotide encoding the target protein; and
b) a second polynucleotide encoding an L protein of Encephalomyocarditis virus (EMCV), optionally wherein the L protein shares at least 90% identity to SEQ ID NO: 2, and
wherein the first polynucleotide and the second polynucleotide are operatively linked to one or more promoters.

70. A system for recombinant expression of a target protein in eukaryotic cells, comprising one or more vectors, the one or more vectors comprising:
a) a first polynucleotide encoding the target protein; and
b) a second polynucleotide encoding a L protein of Theiler's virus, optionally wherein the L protein shares at least 90% identity to SEQ ID NO: 1, and
wherein the first polynucleotide and the second polynucleotide are operatively linked to one or more promoters.

71. A system for recombinant expression of a target protein in eukaryotic cells, comprising one or more vectors, the one or more vectors comprising:
a) a first polynucleotide encoding the target protein; and
b) a second polynucleotide encoding a picornavirus 2A protease, optionally wherein the picornavirus 2A protease shares at least 90% identity to SEQ ID NO: 7, and
wherein the first polynucleotide and the second polynucleotide are operatively linked to one or more promoters.

72. A system for recombinant expression of a target protein in eukaryotic cells, comprising one or more vectors, the one or more vectors comprising:
a) a first polynucleotide encoding the target protein; and
b) a second polynucleotide encoding a M protein of Vesicular stomatitis virus (VSV), optionally wherein the M protein shares at least 90% identity to SEQ ID NO: 9, and
wherein the first polynucleotide and the second polynucleotide are operatively linked to one or more promoters.

73. The system of any one of claims 69-72, wherein the target protein is Dopamine receptor 1 (DRD1), optionally wherein the DRD1 comprises an amino acid sequence having at least 90% identity to the amino acid sequence of SEQ ID NO: 19.

74. The system of any one of claims 69-72, wherein the target protein is Cystic fibrosis transmembrane conductance regulator (CFTR), optionally wherein the CFTR comprises an amino acid sequence having at least 90% identity to the amino acid sequence of SEQ ID NO: 18.

75. The system of any one of claims 69-72, wherein the target protein is Cl esterase inhibitor (C1-Inh), optionally wherein the C1-Inh comprises an amino acid sequence having at least 90% identity to the amino acid sequence of SEQ ID NO: 16.

76. The system of any one of claims 69-72, wherein the target protein is ITK, optionally wherein the ITK comprises an amino acid sequence having at least 90% identity to the amino acid sequence of SEQ ID NO: 15.

77. The system of any one of claims 69-72, wherein the target protein is an NADase, optionally wherein the NADase comprises an amino acid sequence having at least 90% identity to the amino acid sequence of SEQ ID NO: 20.
